(19) United States
(12) Patent Application Publication
(10) Pub. No.: US 2023/0125380 A1
(43) Pub. Date: Apr. 27, 2023

(54) Topical Compositions Comprising IRAK4 Inhibitors for Use in Treating Dermatological Conditions Characterised by Inflammation

(71) Applicant: Dermira, Inc., Menlo Park, CA (US)

(72) Inventors: Jamie L. Harden, Menlo Park, CA (US); Hans E.J. Hofland, Menlo Park, CA (US)

(21) Appl. No.: 17/771,777

(22) PCT Filed: Nov. 5, 2020
(86) PCT No.: PCT/US2020/059200

§ 371 Date: Apr. 25, 2022

Related U.S. Application Data
(60) Provisional application No. 62/931,132, filed on Nov. 5, 2019.
(60) Provisional application No. 63/046,143, filed on Jun. 30, 2020.

(51) Int. Cl. A61K 31/4439; A61K 9/00; A61P 29/00; A61P 17/00

(52) U.S. Cl. CPC A61K 31/4439; A61K 9/0014; A61P 29/00; A61P 17/00

(57) ABSTRACT

This disclosure is directed to use of IRAK4 inhibitors in the treatment of dermatological disorders or conditions characterized by inflammation This disclosure is also directed to pharmaceutical compositions comprising an IRAK4 inhibitor and a pharmaceutically acceptable carrier for topical administration

### CROSS-REFERENCE TO RELATED APPLICATIONS

This application claims priority to and the benefit of U.S. Provisional Application Ser. Nos. 62/931,132, filed on Nov. 5, 2019, and 63/046,143, filed on Jun. 30, 2020, the contents of each of which are hereby incorporated by reference in their entireties.

### BACKGROUND

Dermatological disorders associated with overactive innate inflammation, such as rosacea, psoriasis, and atopic dermatitis, are common dermatological conditions affecting many people. For example, rosacea is a common and chronic inflammatory skin disease that affects over 10 million Americans. Rosacea presents with at least one of the following symptoms: flushing (transient redness), non-transient redness, papules, pustules, and telangiectases (visible, small dilated blood vessels). Although the phenotypes of rosacea are clinically heterogeneous, they are all related by the presence of chronic facial skin inflammation. Until recently, the pathophysiology of this disease has been poorly understood and limited to descriptions of factors that exacerbate or improve this disorder. Recent molecular studies suggest that an altered innate immune response is involved in the pathogenesis of the vascular and inflammatory disease seen in patients with rosacea. Currently available treatments for rosacea include vasoconstrictors such as alpha blockers or beta blockers, antibiotics, light therapy, and laser therapy

There is currently a need for a fast-acting, effective and safe dermatological therapy for dermatological disorders that are characterized by inflammation.

### SUMMARY

Described herein are topical compositions comprising inhibitors IRAK4 and TrkA, which may also have low inhibitory activity on VEGFR, and methods for using the IRAK4 inhibitors for the treatment of dermatological disorders or conditions characterized by inflammation, such as rosacea.

In one aspect, the present disclosure provides for a topical composition comprising a dermatologically acceptable excipient and a pharmaceutically effective amount of an IRAK4 inhibitor (e.g., a compound having the following Formula I, II (e.g., Compound 1, et seq.), or III (e.g., Compound 2, et seq.).

In a further aspect, the present disclosure provides for a method for treating a dermatological disorder, the method comprising topically administering to a subject in need thereof a topical composition having a therapeutically effective amount of an IRAK4 inhibitor of Formula I, II (e.g., Compound 1, et seq.), or III (e.g., Compound 2, et seq.); and a dermatologically acceptable excipient.

In a further aspect, the present disclosure provides a method for reducing inflammation in mammalian skin, the method comprising topically administering to the mammalian skin an effective amount of a topical composition including an IRAK4 inhibitor of compound of Formula I, II (e.g., Compound 1, et seq.), or III (e.g., Compound 2, et seq.) to a subject in need thereof.

In a further aspect, the present disclosure provides a method of reducing inflammation and vascular dysfunction in mammalian skin, the method comprising topically administering to the mammalian skin a therapeutically effective amount of a topical composition including an IRAK4 inhibitor a compound of Formula I, II (e.g., Compound 1, et seq.), or III (e.g., Compound 2, et seq.) to a subject in need thereof.

### BRIEF DESCRIPTION OF THE DRAWINGS

FIG. 1 provides the results of the topical screening of IRAK4/TrkA inhibitors in the skin resident immune cell assay.

FIG. 2 shows differentially expressed proteins in lesional rosacea (LS) versus non-lesional rosacea (NL), as measured by OLINK.

FIG. 3 shows a plot comparing the differential expression between: NL versus IL-1β treated NL on the y axis, and NL versus LS on the x axis.

FIGS. 4A and 4B show the RNA (FIG. 4A) and protein levels (FIG. 4B) of MMP-1 following treatment with the compound of Formula (Ia) at a concentration of 1 μM and 0.5 μM, or clobetasol at a concentration of 1 μM.

FIGS. 5A and 5B show the RNA (FIG. 5A) and protein levels (FIG. 5B) of CXCL5 following treatment with the compound of Formula (Ia) at a concentration of 1 μM and 0.5 μM, or clobetasol at a concentration of 1 μM.

FIGS. 6A and 6B show the RNA (FIG. 6A) and protein levels (FIG. 6B) of IL1β following treatment with the compound of Formula (Ia) at a concentration of 1 μM and 0.5 μM, or clobetasol at a concentration of 1 μM.

FIG. 7 shows the protein level of CXCL1 in rosacea non-lesional skin (NL), untreated rosacea lesional skin (LS), and rosacea LS 24 hours following treatment with the compound of Formula (Ia) at a concentration of 1 μM and 0.5 μM, or clobetasol at a concentration of 1 μM, as measured by OLink proteomics.

FIG. 8 shows the protein level of β-NGF in rosacea non-lesional skin (NL), untreated rosacea lesional skin (LS), and rosacea LS 24 hours following treatment with the compound of Formula (Ia) at a concentration of 1 μM and 0.5 μM, or clobetasol at a concentration of 1 μM, as measured by OLink proteomics.

FIG. 9 shows the protein level of LIF in rosacea non-lesional skin (NL), untreated rosacea lesional skin (LS), and rosacea LS 24 hours following treatment with the compound of Formula (Ia) at a concentration of 1 μM and 0.5 μM, or clobetasol at a concentration of 1 μM, as measured by OLink proteomics.

FIG. 10 shows the protein level of TGF-α in rosacea non-lesional skin (NL), untreated rosacea lesional skin (LS), and rosacea LS 24 hours following treatment with the compound of Formula (Ia) at a concentration of 1 μM and 0.5 μM, or clobetasol at a concentration of 1 μM, as measured by OLink proteomics.

FIG. 11 shows the protein level of IL-8 in rosacea non-lesional skin (NL), untreated rosacea lesional skin (LS), and rosacea LS 24 hours following treatment with the compound of Formula (Ia) at a concentration of 1 μM and 0.5 μM, or clobetasol at a concentration of 1 μM, as measured by OLink proteomics.

FIG. 12 shows the protein level of IL-6 in rosacea non-lesional skin (NL), untreated rosacea lesional skin (LS), and rosacea LS 24 hours following treatment with the compound of Formula (Ia) at a concentration of 1 μM and 0.5 μM, or clobetasol at a concentration of 1 μM, as measured by OLink proteomics.

FIGS. 13A-13F show protein expression levels in rosacea non-lesional (NL) skin, rosacea lesional (LS) skin, or rosacea lesional (LS) skin following treatment with the compounds of Formula (Ia), as measured by the MesoScale Discovery (MSD) plate-based immunoassay. Results for IL-5 are shown in FIG. 13A; results for IL-6 are shown in FIG. 13B, results for IL-4 are shown in FIG. 13C, results for IL-10 are shown in FIG. 13D, results for MCP-1 are shown in FIG. 13E, and results for IL-8 are shown in FIG. 13F.

### DETAILED DESCRIPTION

Provided herein are topical compositions for treating dermatological conditions characterized by inflammation. In particular, the pharmaceutical compositions include compounds that are dual inhibitors of interleukin-1 receptor-associated kinase 4 (IRAK4) and tropomyosin receptor kinase A (TrkA). IRAK4 is a protein involved in signalling innate immune responses downstream from Toll-like receptors (except for TLR3) and IL-1 family cytokine receptors (all MyD88-dependent). TrkA is the high affinity catalytic receptor for nerve growth factor (NGF). While not wishing to be bound by theory, inhibition of IRAK4 and TrkA may potently reverse the overactive innate inflammatory state of the skin, as well as reduce skin vascular abnormality and sensitivity. The topical compositions of IRAK4/TrkA inhibitors are particularly capable of addressing all three key components rosacea pathology including innate inflammation, redness, and sensitivity.

### Dual IRAK4 and TrkA Inhibitors

The present disclosure provides compounds having IRAK4 inhibitory properties that also targets TrkA.

In some embodiments, the compound for use in the methods or compositions described herein are compositions according to Formula I:

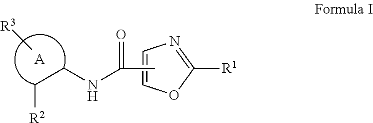

wherein
Ring A is monocyclic heteroaryl;
R¹ is optionally substituted monocyclic or bicyclic heteroaryl;
R² is —CONH₂, —CONH—R⁰, —CONH—R⁰⁰—OH, phenyl, oxadiazolyl, tetrazolyl or the like;
R³ is H, hetero cycloalkyl (optionally substituted by R⁰, halogen and the like) or the like;
R⁰ is lower alkyl; and
R⁰⁰ is lower alkylene,
in free or pharmaceutically acceptable salt form, including its racemates, enantiomers and diastereomers.

In some embodiments, the present disclosure provides for a topical composition comprising a compound [Compound 1] according to Formula II:

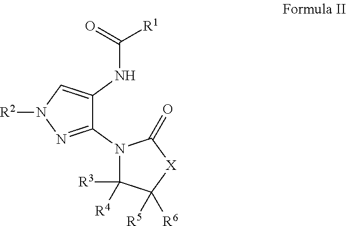

wherein:
R¹ is an optionally substituted aromatic heterocyclic group or an optionally substituted C₆₋₁₄ aryl group;
R² is a hydrogen atom or a substituent;
R³ and R⁴ are independently a hydrogen atom or a substituent, or R³ and R⁴ in combination optionally form an optionally substituted ring;
R⁵ and R⁶ are independently a hydrogen atom or a substituent, or R⁵ and R⁶ in combination optionally form an optionally substituted ring;
X is CR⁷R⁸, NR⁹, O or S;
R⁷ and R⁸ are independently a hydrogen atom or a substituent, or R⁷ and R⁸ in combination optionally form an optionally substituted ring; and
R⁹ is a hydrogen atom or a substituent,
in free or pharmaceutically acceptable salt form, including its racemates, enantiomers and diastereomers.

The disclosure further provides a compound of Formula II as follows:
1.1 Compound 1, wherein:
   R1 is an aromatic, heterocyclic group or a C₆₋₁₄ aryl group, each of which is optionally substituted by 1 to 3 substituents selected from a halogen atom, an optionally substituted C₁₋₆ alkyl group, an optionally substituted C₆₋₁₄ aryl group, an optionally substituted heterocyclic group, a C₃₋₁₀ cycloalkylsulfonyl group, a C₁₋₆ alkyl-carbonyl group, an aromatic heterocyclylsulfonyl group and a halogenated sulfanyl group;
   R2 is an optionally substituted C₁₋₆ alkyl group, an optionally substituted C₃₋₁₀ cycloalkyl group or an optionally substituted non-aromatic heterocyclic group;
   R3 and R4 are independently a hydrogen atom or an optionally substituted C₁₋₆ alkyl group;
   R5 and R6 are independently (1) a hydrogen atom, (2) a hydroxy group, (3) an optionally substituted C₁₋₆ alkyl group, (4) an optionally substituted C₁₋₆ alkoxy group, (5) an amino group optionally mono- or di-substituted by substituent(s) selected from (i) an optionally substituted C₁₋₆ alkyl group, (ii) an optionally substituted C₁₋₆ alkyl-carbonyl group, and (iii) an optionally substituted C1-6 alkylsulfonyl group, (6) an optionally substituted non-aromatic heterocyclic group, (7) a carboxy group, or (8) a carbamoyl group optionally mono- or di-substituted by C₁₋₆ alkyl group(s), or R5 and R6 in combination optionally form an optionally substituted non-aromatic heterocycle or an optionally substituted C₃₋₁₀ cycloalkane;
   X is CR⁷R⁸, NR⁹, O or S;
   R⁷ and R⁸ are independently a hydrogen atom, a cyano group, an optionally substituted C₁₋₆ alkyl group or a hydroxy group, or R⁷ and R⁸ in combination optionally form an optionally substituted C₃₋₁₀ cycloalkane or an optionally substituted non-aromatic heterocycle; and
   R⁹ is a hydrogen atom, an optionally substituted C₁₋₆ alkyl group, an optionally substituted C₂₋₆ alkenyl group or an optionally substituted C₇₋₁₆ aralkyl group.
1.2 Compound 1 or 1.1, wherein
   X is CR⁷R⁸ or NR⁹; and
   R3 and R4 are both hydrogen atoms.
1.3 A compound according to Compound 1 or 1.1-1.2, wherein the compound is N-(3-(3-(2-Hydroxyethyl)-2-oxoimidazolidin-1-yl)-1-methyl-1H-pyrazol-4-yl)-2-(2-((2,2,2-trifluoroethyl)amino)pyridin-4-yl)-1,3-oxazole-4-carboxamide or a salt thereof.
1.4 A compound according to Compound 1 or 1.1-1.2, wherein the compound is N-(1-Methyl-3-(2-oxoimidazolidin-1-yl)-1H-pyrazol-4-yl)-2-(2-((2,2,2-trifluoroethyl)amino)pyridin-4-yl)-1,3-oxazole-4-carboxamide or a salt thereof.
1.5 A compound according to Compound 1 or 1.1-1.2, wherein the compound is N-(1-Methyl-3-((3S)-3-methyl-2-oxopyrrolidin-1-yl)-1H-pyrazol-4-yl)-2-(2-((2,2,2-trifluoroethyl)amino)pyridin-4-yl)-1,3-oxazole-4-carboxamide or a salt thereof.

Each symbol in formula II is explained below.

R¹ is an optionally substituted aromatic heterocyclic group or an optionally substituted C₆₋₁₄ aryl group.

The “aromatic heterocyclic group” of the “optionally substituted aromatic heterocyclic group” and the “C₆₋₁₄ aryl group” of the “optionally substituted C₆₋₁₄ aryl group” for R¹ each optionally has 1 to 3 substituents at substitutable position(s). When the number of the substituents is plural, the respective substituents may be the same or different.

In one embodiment, examples of the “substituent” for the “aromatic heterocyclic group” of the “optionally substituted aromatic heterocyclic group” and the “C₆₋₁₄ aryl group” of the “optionally substituted C₆₋₁₄ aryl group” for R¹ include a halogen atom, a cyano group, a nitro group, an optionally substituted hydrocarbon group, an optionally substituted heterocyclic group (the “heterocyclic group” optionally has substituent(s) selected from Substituent Group A (the substituent is optionally further substituted by substituent(s) selected from Substituent Group A)), an acyl group, an optionally substituted amino group, an optionally substituted carbamoyl group, an optionally substituted thiocarbamoyl group, an optionally substituted sulfamoyl group, an optionally substituted hydroxy group, an optionally substituted sulfanyl (SH) group, and an optionally substituted silyl group.

Preferable examples of the “substituent” for the “aromatic heterocyclic group” of the “optionally substituted aromatic heterocyclic group” and the “C₆₋₁₄ aryl group” of the “optionally substituted C₆₋₁₄ aryl group” for R¹ include
(1) an optionally substituted hydrocarbon group (e.g., a hydrocarbon group optionally having substituent(s) selected from Substituent Group A),
(2) an optionally substituted heterocyclic group (e.g., a heterocyclic group optionally having substituent(s) selected from Substituent Group A (the substituent is optionally further substituted by substituent(s) selected from Substituent Group A)), and
(3) an acyl group.

In another embodiment, examples of the “substituent” for the “aromatic heterocyclic group” of the “optionally substituted aromatic heterocyclic group” and the “C₆₋₁₄ aryl group” of the “optionally substituted C₆₋₁₄ aryl group” for R¹ include a halogen atom, a cyano group, a nitro group, an optionally substituted hydrocarbon group, an optionally substituted heterocyclic group (the “heterocyclic group” optionally has substituent(s) selected from Substituent Group A and a thioxo group (the substituent is optionally further substituted by substituent(s) selected from Substituent Group A, an azido group and a mono- or di-C₁₋₆ alkylamino group (the alkyl is substituted by substituent(s) selected from a C₃₋₁₀ cycloalkyl group and a halogen atom))), an acyl group, an optionally substituted amino group, an optionally substituted carbamoyl group, an optionally substituted thiocarbamoyl group, an optionally substituted sulfamoyl group, an optionally substituted hydroxy group, an optionally substituted sulfanyl (SH) group, and an optionally substituted silyl group.

Preferable examples of the “substituent” for the “aromatic heterocyclic group” of the “optionally substituted aromatic heterocyclic group” and the “C₆₋₁₄ aryl group” of the “optionally substituted C₆₋₁₄ aryl group” for R¹ include
(1) a halogen atom,
(2) an optionally substituted hydrocarbon group (e.g., a hydrocarbon group optionally having substituent(s) selected from Substituent Group A),
(3) an optionally substituted heterocyclic group (e.g., a heterocyclic group optionally having substituent(s) selected from Substituent Group A and a thioxo group (the substituent is optionally further substituted by substituent(s) selected from Substituent Group A, an azido group and a mono- or di-C₁₋₆ alkylamino group (the alkyl is substituted by substituent(s) selected from a C₃₋₁₀ cycloalkyl group and a halogen atom))),
(4) an acyl group, and
(5) an optionally substituted sulfanyl (SH) group.

In one embodiment, R¹ is preferably an aromatic heterocyclic group (preferably a 5- to 14-membered aromatic heterocyclic group) or a C₆₋₁₄ aryl group, each of which is optionally substituted by 1 to 3 substituents selected from
(1) an optionally substituted C₁₋₆ alkyl group (e.g., a C₁₋₆ alkyl group optionally having substituent(s) selected from Substituent Group A),
(2) an optionally substituted C₆₋₁₄ aryl group (e.g., a C₆₋₁₄ aryl group optionally having substituent(s) selected from Substituent Group A),
(3) an optionally substituted heterocyclic group (e.g., a heterocyclic group optionally having substituent(s) selected from Substituent Group A (the substituent is optionally further substituted by substituent(s) selected from Substituent Group A)),
(4) a C₃₋₁₀ cycloalkylsulfonyl group,
(5) a C₁₋₆ alkyl-carbonyl group, and
(6) an aromatic heterocyclylsulfonyl group (preferably a 5- to 14-membered aromatic heterocyclylsulfonyl group).

R¹ is more preferably an aromatic heterocyclic group (preferably a 5- to 14-membered aromatic heterocyclic group, more preferably a 5- to 6-membered monocyclic aromatic heterocyclic group, a 8- to 14-membered fused polycyclic aromatic heterocyclic group) (e.g., oxazolyl, thiazolyl, thienyl, pyrazolyl, pyridyl, imidazopyridyl (e.g., imidazo[1,5-a]pyridyl), imidazopyridazinyl (e.g., imidazo[1,2-b]pyridazinyl), pyrazolopyrimidinyl (e.g., pyrazolo[1,5-a]pyrimidinyl)), or a C₆₋₁₄ aryl group (e.g., phenyl), each of which is optionally substituted by 1 to 3 substituents selected from
(1) a C₁₋₆ alkyl group (e.g., methyl, ethyl, isopropyl) optionally substituted by 1 to 3 substituents selected from
   (i) a halogen atom (e.g., a fluorine atom), and
   (ii) a hydroxy group,
(2) a C₅₋₁₄ aryl group (e.g., phenyl) optionally substituted by 1 to 3 halogen atoms (e.g., a fluorine atom),
(3) an aromatic heterocyclic group (preferably a 5- to 14-membered aromatic heterocyclic group, more preferably a 5- to 6-membered monocyclic aromatic heterocyclic group) (e.g., pyridyl, thienyl) optionally substituted by amino group(s) optionally mono- or di-substituted by C₁₋₆ alkyl group(s) (e.g., ethyl) optionally substituted by 1 to 3 halogen atoms (e.g., a fluorine atom),
(4) a non-aromatic heterocyclic group (preferably a 3- to 14-membered non-aromatic heterocyclic group, more preferably a 3- to 8-membered monocyclic non-aromatic heterocyclic group) (e.g., morpholinyl) optionally substituted by 1 to 3 C₁₋₆ alkyl groups (e.g., methyl),
(5) a C₃₋₁₀ cycloalkylsulfonyl group (e.g., cyclopentylsulfonyl),
(6) a C₁₋₆ alkyl-carbonyl group (e.g., acetyl), and
(7) an aromatic heterocyclylsulfonyl group (preferably a 5- to 14-membered aromatic heterocyclylsulfonyl group, more preferably a 5- to 6-membered monocyclic aromatic heterocyclylsulfonyl group) (e.g., thiazolylsulfonyl).

R¹ is further more preferably an aromatic heterocyclic group (preferably a 5- to 14-membered aromatic heterocyclic group, more preferably a 5- to 6-membered monocyclic aromatic heterocyclic group, a 8- to 14-membered fused polycyclic aromatic heterocyclic group) (e.g., oxazolyl, thiazolyl, thienyl, pyrazolyl, pyridyl, imidazopyridyl (e.g., imidazo[1,5-a]pyridyl), imidazopyridazinyl (e.g., imidazo[1,2-b]pyridazinyl), pyrazolopyrimidinyl (e.g., pyrazolo[1,5-a]pyrimidinyl)) optionally substituted by 1 to 3 substituents selected from
(i) a C₁₋₆ alkyl group (e.g., methyl, ethyl, isopropyl) optionally substituted by 1 to 3 substituents selected from
   (a) a halogen atom (e.g., a fluorine atom), and
   (b) a hydroxy group,
(ii) a C₆₋₁₄ aryl group (e.g., phenyl) optionally substituted by 1 to 3 halogen atoms (e.g., a fluorine atom),
(iii) an aromatic heterocyclic group (preferably a 5- to 14-membered aromatic heterocyclic group, more preferably a 5- to 6-membered monocyclic aromatic heterocyclic group) (e.g., pyridyl, thienyl) optionally substituted by amino group(s) optionally mono- or di-substituted by C₁₋₆ alkyl group(s) (e.g., ethyl) optionally substituted by 1 to 3 halogen atoms (e.g., a fluorine atom),
(iv) a non-aromatic heterocyclic group (preferably a 3- to 14-membered non-aromatic heterocyclic group, more preferably a 3- to 8-membered monocyclic non-aromatic heterocyclic group) (e.g., morpholinyl) optionally substituted by 1 to 3 C₁₋₆ alkyl groups (e.g., methyl),
(v) a C₃₋₁₀ cycloalkylsulfonyl group (e.g., cyclopentylsulfonyl), and
(vi) a C₁₋₆ alkyl-carbonyl group (e.g., acetyl), or
(2) a C₆₋₁₄ aryl group (e.g., phenyl) optionally substituted by 1 to 3 substituents selected from
(i) a C₁₋₆ alkyl group (e.g., methyl),
(ii) a C₃₋₁₀ cycloalkylsulfonyl group (e.g., cyclopentylsulfonyl), and
(iii) an aromatic heterocyclylsulfonyl group (preferably a 5- to 14-membered aromatic heterocyclylsulfonyl group, more preferably a 5- to 6-membered monocyclic aromatic heterocyclylsulfonyl group) (e.g., thiazolylsulfonyl).

R¹ is particularly preferably an aromatic heterocyclic group (preferably a 5- to 14-membered aromatic heterocyclic group, more preferably a 5- to 6-membered monocyclic aromatic heterocyclic group) (e.g., oxazolyl) optionally substituted by aromatic heterocyclic group(s) (preferably a 5- to 14-membered aromatic heterocyclic group, more preferably a 5- to 6-membered monocyclic aromatic heterocyclic group) (e.g., pyridyl) optionally substituted by amino group(s) optionally mono- or di-substituted by C₁₋₆ alkyl group(s) (e.g., methyl, ethyl) optionally substituted by 1 to 3 halogen atoms (e.g., a fluorine atom).

In another embodiment, R¹ is more preferably an aromatic heterocyclic group (preferably a 5- to 14-membered aromatic heterocyclic group, more preferably a 5- to 6-membered monocyclic aromatic heterocyclic group, a 8- to 14-membered fused polycyclic aromatic heterocyclic group) (e.g., oxazolyl, thiazolyl, thienyl, pyrazolyl, pyridyl, imidazopyridyl (e.g., imidazo[1,5-a]pyridyl), imidazopyridazinyl (e.g., imidazo[1,2-b]pyridazinyl), pyrazolopyrimidinyl (e.g., pyrazolo[1,5-a]pyrimidinyl)), or a C₆₋₁₄ aryl group (e.g., phenyl), each of which is optionally substituted by 1 to 3 substituents selected from
(1) a C₁₋₆ alkyl group (e.g., methyl, ethyl, isopropyl) optionally substituted by 1 to 3 substituents selected from
   (i) a halogen atom (e.g., a fluorine atom), and
   (ii) a hydroxy group,
(2) a C₆₋₁₄ aryl group (e.g., phenyl) optionally substituted by 1 to 3 halogen atoms (e.g., a fluorine atom),
(3) an aromatic heterocyclic group (preferably a 5- to 14-membered aromatic heterocyclic group, more preferably a 5- to 6-membered monocyclic aromatic heterocyclic group) (e.g., pyridyl, thienyl) optionally substituted by amino group(s) optionally mono- or di-substituted by C₁₋₆ alkyl group(s) (e.g., methyl, ethyl) optionally substituted by 1 to 3 substituents selected from
   (i) a halogen atom (e.g., a fluorine atom), and
   (ii) a C₃₋₁₀ cycloalkyl group (e.g., cyclopropyl),
(4) a non-aromatic heterocyclic group (preferably a 3- to 14-membered non-aromatic heterocyclic group, more preferably a 3- to 8-membered monocyclic non-aromatic heterocyclic group) (e.g., morpholinyl) optionally substituted by 1 to 3 C₁₋₆ alkyl groups (e.g., methyl),
(5) a C₃₋₁₀ cycloalkylsulfonyl group (e.g., cyclopentylsulfonyl),
(6) a C₁₋₆ alkyl-carbonyl group (e.g., acetyl), and
(7) an aromatic heterocyclylsulfonyl group (preferably a 5- to 14-membered aromatic heterocyclylsulfonyl group, more preferably a 5- to 6-membered monocyclic aromatic heterocyclylsulfonyl group) (e.g., thiazolylsulfonyl).

R¹ is further more preferably an aromatic heterocyclic group (preferably a 5- to 14-membered aromatic heterocyclic group, more preferably a 5- to 6-membered monocyclic aromatic heterocyclic group, a 8- to 14-membered fused polycyclic aromatic heterocyclic group) (e.g., oxazolyl, thiazolyl, thienyl, pyrazolyl, pyridyl, imidazopyridyl (e.g., imidazo[1,5-a]pyridyl), imidazopyridazinyl (e.g., imidazo[1,2-b]pyridazinyl), pyrazolopyrimidinyl (e.g., pyrazolo[1,5-a]pyrimidinyl)) optionally substituted by 1 to 3 substituents selected from
(i) a C₁₋₆ alkyl group (e.g., methyl, ethyl, isopropyl) optionally substituted by 1 to 3 substituents selected from
   (a) a halogen atom (e.g., a fluorine atom), and
   (b) a hydroxy group,
(ii) a C₆₋₁₄ aryl group (e.g., phenyl) optionally substituted by 1 to 3 halogen atoms (e.g., a fluorine atom),
(iii) an aromatic heterocyclic group (preferably a 5- to 14-membered aromatic heterocyclic group, more preferably a 5- to 6-membered monocyclic aromatic heterocyclic group) (e.g., pyridyl, thienyl) optionally substituted by amino group(s) optionally mono- or di-substituted by C₁₋₆ alkyl group(s) (e.g., methyl, ethyl) optionally substituted by 1 to 3 substituents selected from
   (a) a halogen atom (e.g., a fluorine atom), and
   (b) a C₃₋₁₀ cycloalkyl group (e.g., cyclopropyl),
(iv) a non-aromatic heterocyclic group (preferably a 3- to 14-membered non-aromatic heterocyclic group, more preferably a 3- to 8-membered monocyclic non-aromatic heterocyclic group) (e.g., morpholinyl) optionally substituted by 1 to 3 C₁₋₆ alkyl groups (e.g., methyl),
(v) a C₃₋₁₀ cycloalkylsulfonyl group (e.g., cyclopentylsulfonyl), and
(vi) a C₁₋₅ alkyl-carbonyl group (e.g., acetyl), or
(2) a C₆₋₁₄ aryl group (e.g., phenyl) optionally substituted by 1 to 3 substituents selected from
(i) a C₁₋₆ alkyl group (e.g., methyl),
(ii) a C₃₋₁₀ cycloalkylsulfonyl group (e.g., cyclopentylsulfonyl), and
(iii) an aromatic heterocyclylsulfonyl group (preferably a 5- to 14-membered aromatic heterocyclylsulfonyl group, more preferably a 5- to 6-membered monocyclic aromatic heterocyclylsulfonyl group) (e.g., thiazolylsulfonyl).

In yet another embodiment, R¹ is preferably an aromatic heterocyclic group (preferably a 5- to 14-membered aromatic heterocyclic group) or a C₆₋₁₄ aryl group, each of which is optionally substituted by 1 to 3 substituents selected from
(1) a halogen atom,
(2) an optionally substituted C₁₋₆ alkyl group (e.g., a C₁₋₆ alkyl group optionally having substituent(s) selected from Substituent Group A),
(3) an optionally substituted C₆₋₁₄ aryl group (e.g., a C₆₋₁₄ aryl group optionally having substituent(s) selected from Substituent Group A),
(4) an optionally substituted heterocyclic group (e.g., a heterocyclic group optionally having substituent(s) selected from Substituent Group A and a thioxo group (the substituent is optionally further substituted by substituent(s) selected from Substituent Group A, an azido group and a mono- or di-C₁₋₆ alkylamino group (the alkyl is substituted by substituent(s) selected from a C₃₋₁₀ cycloalkyl group and a halogen atom))),
(5) a C₃₋₁₀ cycloalkylsulfonyl group,
(6) a C₁₋₆ alkyl-carbonyl group,
(7) an aromatic heterocyclylsulfonyl group (preferably a 5- to 14-membered aromatic heterocyclylsulfonyl group), and
(8) a halogenated sulfanyl group.

R¹ is more preferably an aromatic heterocyclic group (preferably a 5- to 14-membered aromatic heterocyclic group, more preferably a 5- to 6-membered monocyclic aromatic heterocyclic group, a 8- to 14-membered fused polycyclic aromatic heterocyclic group) (e.g., oxazolyl, thiazolyl, thienyl, pyrazolyl, pyridyl, imidazopyridyl (e.g., imidazo[1,5-a]pyridyl), imidazopyridazinyl (e.g., imidazo[1,2-b]pyridazinyl), pyrazolopyrimidinyl (e.g., pyrazolo[1,5-a]pyrimidinyl)), or a C₆₋₁₄ aryl group (e.g., phenyl), each of which is optionally substituted by 1 to 3 substituents selected from
(1) a halogen atom (e.g., a fluorine atom),
(2) a C₁₋₆ alkyl group (e.g., methyl, ethyl, isopropyl) optionally substituted by 1 to 3 substituents selected from
   (i) a halogen atom (e.g., a fluorine atom), and
   (ii) a hydroxy group,
(3) a C₆₋₁₄ aryl group (e.g., phenyl) optionally substituted by 1 to 3 halogen atoms (e.g., a fluorine atom),
(4) an aromatic heterocyclic group (preferably a 5- to 14-membered aromatic heterocyclic group, more preferably a 5- to 6-membered monocyclic aromatic heterocyclic group, a 8- to 14-membered fused polycyclic aromatic heterocyclic group) (e.g., pyridyl, thienyl, pyrimidinyl, imidazolyl, pyrazolyl, tetrazolyl, benzimidazolyl (e.g., 1H-benzimidazolyl), thiazolyl) optionally substituted by 1 to 3 substituents selected from
   (i) an amino group optionally mono- or di-substituted by C₁₋₆ alkyl group(s) (e.g., methyl, ethyl) optionally substituted by 1 to 3 substituents selected from
      (a) a halogen atom (e.g., a fluorine atom), and
      (b) a C₃₋₁₀ cycloalkyl group (e.g., cyclopropyl),
   (ii) a halogen atom (e.g., a chlorine atom),
   (iii) a C₁₋₆ alkoxy group (e.g., methoxy),
   (iv) a cyano group,
   (v) a C₁₋₆ alkyl group (e.g., methyl, ethyl) optionally substituted by 1 to 3 substituents selected from
      (a) an azido group,
      (b) an amino group optionally mono- or di-substituted by C₁₋₆ alkyl group(s) (e.g., methyl, ethyl) optionally substituted by 1 to 3 substituents selected from a halogen atom (e.g., a fluorine atom) and a C₃₋₁₀ cycloalkyl group (e.g., cyclopropyl),
      (c) a hydroxy group, and
      (d) a halogen atom (e.g., a fluorine atom),
   (vi) a formyl group,
   (vii) a carboxy group,
   (viii) a carbamoyl group,
   (ix) a C₃₋₁₀ cycloalkyl group (e.g., cyclopropyl), and
   (x) a non-aromatic heterocyclic group (preferably a 3- to 14-membered non-aromatic heterocyclic group, more preferably a 3- to 8-membered monocyclic non-aromatic heterocyclic group) (e.g., dioxolanyl (e.g., 1,3-dioxolanyl)),
(5) a non-aromatic heterocyclic group (preferably a 3- to 14-membered non-aromatic heterocyclic group, more preferably a 3- to 8-membered monocyclic non-aromatic heterocyclic group, a 9- to 14-membered fused polycyclic non-aromatic heterocyclic group, a 7- to 14-membered spiro heterocyclic group) (e.g., morpholinyl, dihydropyranyl (e.g., 3,6-dihydro-2H-pyranyl), tetrahydropyranyl, dihydropyridyl (e.g., 1,2-dihydropyridyl), dihydrobenzofuranyl (e.g., 2,3-dihydrobenzofuranyl), imidazolidinyl, pyrrolidinyl, dihydroisoxazolyl (e.g., 4,5-dihydroisoxazolyl), dihydropyrrolopyrazolyl (e.g., 5,6-dihydropyrrolo[3,4-c]pyrazolyl), piperazinyl, triazaspirononyl (e.g., 1,3,7-triazaspiro[4.4]nonyl), thiadiazaspirononyl (e.g., 7-thia-1,3-diazaspiro[4.4]nonyl), dioxidothiadiazaspirononyl (e.g., 7,7-dioxido-7-thia-1,3-diazaspiro[4.4]nonyl)) optionally substituted by 1 to 3 substituents selected from
   (i) a C₁₋₆ alkyl group (e.g., methyl, ethyl) optionally substituted by 1 to 3 substituents selected from
      (a) a hydroxy group,
      (b) an amino group optionally mono- or di-substituted by C₁₋₆ alkyl group(s) (e.g., methyl),
      (c) a cyano group, and
      (d) a C₆₋₁₄ aryl group (e.g., phenyl),
   (ii) an oxo group,
   (iii) a hydroxy group,
   (iv) a carbamoyl group, and
   (v) a thioxo group,
(6) a C₃₋₁₀ cycloalkylsulfonyl group (e.g., cyclopentylsulfonyl),
(7) a C₁₋₆ alkyl-carbonyl group (e.g., acetyl),
(8) an aromatic heterocyclylsulfonyl group (preferably a 5- to 14-membered aromatic heterocyclylsulfonyl group, more preferably a 5- to 6-membered monocyclic aromatic heterocyclylsulfonyl group) (e.g., thiazolylsulfonyl), and
(9) a halogenated thio group (e.g., pentafluorothio).

R¹ is further more preferably an aromatic heterocyclic group (preferably a 5- to 14-membered aromatic heterocyclic group, more preferably a 5- to 6-membered monocyclic aromatic heterocyclic group, a 8- to 14-membered fused polycyclic aromatic heterocyclic group) (e.g., oxazolyl, thiazolyl, thienyl, pyrazolyl, pyridyl, imidazopyridyl (e.g., imidazo[1,5-a]pyridyl), imidazopyridazinyl (e.g., imidazo[1,2-b]pyridazinyl), pyrazolopyrimidinyl (e.g., pyrazolo[1,5-a]pyrimidinyl)) optionally substituted by 1 to 3 substituents selected from
(i) a C₁₋₆ alkyl group (e.g., methyl, ethyl, isopropyl) optionally substituted by 1 to 3 substituents selected from
   (a) a halogen atom (e.g., a fluorine atom), and
   (b) a hydroxy group,
(ii) a C₆₋₁₄ aryl group (e.g., phenyl) optionally substituted by 1 to 3 halogen atoms (e.g., a fluorine atom),
(iii) an aromatic heterocyclic group (preferably a 5- to 14-membered aromatic heterocyclic group, more preferably a 5- to 6-membered monocyclic aromatic heterocyclic group) (e.g., pyridyl, thienyl, pyrimidinyl, pyrazolyl, thiazolyl, imidazolyl) optionally substituted by 1 to 3 substituents selected from
   (a) an amino group optionally mono- or di-substituted by C₁₋₆ alkyl group(s) (e.g., methyl, ethyl) optionally substituted by 1 to 3 substituents selected from
      (I) a halogen atom (e.g., a fluorine atom), and
      (II) a C3-10 cycloalkyl group (e.g., cyclopropyl),
   (b) a halogen atom (e.g., a chlorine atom),
   (c) a C₁₋₆ alkoxy group (e.g., methoxy),
   (d) a cyano group,
   (e) a C₁₋₆ alkyl group (e.g., methyl, ethyl) optionally substituted by 1 to 3 substituents selected from
      (I) an azido group,
      (II) an amino group optionally mono- or di-substituted by C₁₋₆ alkyl group(s) (e.g., methyl, ethyl) optionally substituted by 1 to 3 substituents selected from a halogen atom (e.g., a fluorine atom) and a C₃₋₁₀ cycloalkyl group (e.g., cyclopropyl),
      (III) a hydroxy group, and
      (IV) a halogen atom (e.g., a fluorine atom),
   (f) a formyl group,
   (g) a carboxy group,
   (h) a carbamoyl group,
   (i) a C₃₋₁₀ cycloalkyl group (e.g., cyclopropyl), and
   (j) a non-aromatic heterocyclic group (preferably a 3- to 14-membered non-aromatic heterocyclic group, more preferably a 3- to 8-membered monocyclic non-aromatic heterocyclic group) (e.g., dioxolanyl (e.g., 1,3-dioxolanyl)),
(iv) a non-aromatic heterocyclic group (preferably a 3- to 14-membered non-aromatic heterocyclic group, more preferably a 3- to 8-membered monocyclic non-aromatic heterocyclic group, a 9- to 14-membered fused polycyclic non-aromatic heterocyclic group, a 7- to 14-membered Spiro heterocyclic group) (e.g., morpholinyl, dihydropyranyl (e.g., 3,6-dihydro-2H-pyranyl), tetrahydropyranyl, dihydropyridyl (e.g., 1,2-dihydropyridyl), dihydrobenzofuranyl (e.g., 2,3-dihydrobenzofuranyl), imidazolidinyl, pyrrolidinyl, dihydroisoxazolyl (e.g., 4,5-dihydroisoxazolyl), dihydropyrrolopyrazolyl (e.g., 5,6-dihydropyrrolo[3,4-c]pyrazolyl), piperazinyl, triazaspirononyl (e.g., 1,3,7-triazaspiro[4.4]nonyl), thiadiazaspirononyl (e.g., 7-thia-1,3-diazaspiro[4.4]nonyl), dioxidothiadiazaspirononyl (e.g., 7,7-dioxido-7-thia-1,3-diazaspiro[4.4]nonyl)) optionally substituted by 1 to 3 substituents selected from
   (a) a C₁₋₆ alkyl group (e.g., methyl, ethyl) optionally substituted by 1 to 3 substituents selected from
      (I) a hydroxy group,
      (II) an amino group optionally mono- or di-substituted by C₁₋₆ alkyl group(s) (e.g., methyl),
      (III) a cyano group, and
      (IV) a C₆₋₁₄ aryl group (e.g., phenyl),
   (b) an oxo group,
   (c) a hydroxy group,
   (d) a carbamoyl group, and
   (e) a thioxo group,
(v) a C₃₋₁₀ cycloalkylsulfonyl group (e.g., cyclopentylsulfonyl), and
(vi) a C₁₋₆ alkyl-carbonyl group (e.g., acetyl), or
(2) a C₆₋₁₄ aryl group (e.g., phenyl) optionally substituted by 1 to 3 substituents selected from
(i) a halogen atom (e.g., a fluorine atom),
(ii) a C₁₋₆ alkyl group (e.g., methyl),
(iii) an aromatic heterocyclic group (preferably a 5- to 14-membered aromatic heterocyclic group, more preferably a 5- to 6-membered monocyclic aromatic heterocyclic group, a 8- to 14-membered fused polycyclic aromatic heterocyclic group) (e.g., imidazolyl, pyrazolyl, tetrazolyl, benzimidazolyl (e.g., 1H-benzimidazolyl)),
(iv) a C₃₋₁₀ cycloalkylsulfonyl group (e.g., cyclopentylsulfonyl),
(v) an aromatic heterocyclylsulfonyl group (preferably a 5- to 14-membered aromatic heterocyclylsulfonyl group, more preferably a 5- to 6-membered monocyclic aromatic heterocyclylsulfonyl group) (e.g., thiazolylsulfonyl),
(vi) a halogenated thio group (e.g., pentafluorothio), and
(vii) a non-aromatic heterocyclic group (preferably a 3- to 14-membered non-aromatic heterocyclic group, more preferably a 3- to 8-membered monocyclic non-aromatic heterocyclic group, a 7- to 14-membered Spiro heterocyclic group) (e.g., imidazolidinyl, triazaspirononyl (e.g., 1,3,7-triazaspiro[4.4]nonyl)) optionally substituted by 1 to 3 substituents selected from
   (a) a C₁₋₆ alkyl group (e.g., methyl) optionally substituted by 1 to 3 amino groups, and
   (b) an oxo group.

R¹ is still more preferably an aromatic heterocyclic group (preferably a 5- to 14-membered aromatic heterocyclic group, more preferably a 5- to 6-membered monocyclic aromatic heterocyclic group) (e.g., oxazolyl, pyridyl; pyrazolyl) optionally substituted by 1 to 3 substituents selected from
(i) a C₁₋₆ alkyl group (e.g., methyl) optionally substituted by 1 to 3 halogen atoms (e.g., a fluorine atom),
(ii) an aromatic heterocyclic group (preferably a 5- to 14-membered aromatic heterocyclic group, more preferably a 5- to 6-membered monocyclic aromatic heterocyclic group) (e.g., pyridyl, pyrazolyl) optionally substituted by 1 to 3 substituents selected from
   (a) an amino group optionally mono- or di-substituted by C₁₋₆ alkyl group(s) (e.g., methyl, ethyl) optionally substituted by 1 to 3 substituents selected from
      (I) a halogen atom (e.g., a fluorine atom), and
      (II) a C₃₋₁₀ cycloalkyl group (e.g., cyclopropyl), and
   (b) a C₁₋₆ alkyl group (e.g., methyl) optionally substituted by 1 to 3 amino groups, and
(iii) a non-aromatic heterocyclic group (preferably a 3- to 14-membered non-aromatic heterocyclic group, more preferably a 3- to 8-membered monocyclic non-aromatic heterocyclic group, a 7- to 14-membered spiro heterocyclic group) (e.g., imidazolidinyl, triazaspirononyl (e.g., 1,3,7-triazaspiro[4.4]nonyl)) optionally substituted by 1 to 3 substituents selected from
   (a) a C₁₋₆ alkyl group (e.g., methyl) optionally substituted by 1 to 3 amino groups, and
   (b) an oxo group, or
   (2) a C₆₋₁₄ aryl group (e.g., phenyl) optionally substituted by 1 to 3 C₁₋₆ alkyl groups (e.g., methyl).

R¹ is particularly preferably an aromatic heterocyclic group (preferably a 5- to 14-membered aromatic heterocyclic group, more preferably a 5- to 6-membered monocyclic aromatic heterocyclic group) (e.g., oxazolyl) optionally substituted by aromatic heterocyclic group(s) (preferably a 5- to 14-membered aromatic heterocyclic group, more preferably a 5- to 6-membered monocyclic aromatic heterocyclic group) (e.g., pyridyl) optionally substituted by amino group(s) optionally mono- or di-substituted by C₁₋₆ alkyl group(s) (e.g., methyl, ethyl) optionally substituted by 1 to 3 substituents selected from

(1) a halogen atom (e.g., a fluorine atom), and

(2) a C₃₋₁₀ cycloalkyl group (e.g., cyclopropyl).

R² is a hydrogen atom or a substituent.

In one embodiment, examples of the “substituent” for R² include those similar to the “substituent” exemplified in the present specification.

The “substituent” for R² is preferably an optionally substituted hydrocarbon group (e.g., a hydrocarbon group optionally having substituent(s) selected from Substituent Group A), more preferably an optionally substituted C₁₋₆ alkyl group (e.g., a C₁₋₆ alkyl group optionally having substituent(s) selected from Substituent Group A).

In another embodiment, examples of the “substituent” for R² include a halogen atom, a cyano group, a nitro group, an optionally substituted hydrocarbon group (the “hydrocarbon group” optionally has substituent(s) selected from Substituent Group A, and a non-aromatic heterocyclic group having oxo group(s)), an optionally substituted heterocyclic group, an acyl group, an optionally substituted amino group, an optionally substituted carbamoyl group, an optionally substituted thiocarbamoyl group, an optionally substituted sulfamoyl group, an optionally substituted hydroxy group, an optionally substituted sulfanyl (SH) group, and an optionally substituted silyl group.

The “substituent” for R² is preferably an optionally substituted hydrocarbon group (e.g., a hydrocarbon group optionally having substituent(s) selected from Substituent Group A, and a non-aromatic heterocyclic group having oxo group(s)), or an optionally substituted heterocyclic group (e.g., a heterocyclic group optionally having substituent(s) selected from Substituent Group A), more preferably
(1) an optionally substituted C₁₋₆ alkyl group (e.g., a C₁₋₆ alkyl group optionally having substituent(s) selected from Substituent Group A, and a non-aromatic heterocyclic group (preferably a 3- to 14-membered non-aromatic heterocyclic group) having oxo group(s)),
(2) an optionally substituted C₃₋₁₀ cycloalkyl group (e.g., a C₃₋₁₀ cycloalkyl group optionally having substituent(s) selected from Substituent Group A), or
(3) an optionally substituted non-aromatic heterocyclic group (preferably a 3- to 14-membered non-aromatic heterocyclic group) (e.g., a non-aromatic heterocyclic group (preferably a 3- to 14-membered non-aromatic heterocyclic group) optionally having substituent(s) selected from Substituent Group A).

In one embodiment, R² is preferably an optionally substituted C₁₋₆ alkyl group (e.g., a C₁₋₆ alkyl group optionally having substituent(s) selected from Substituent Group A).

In another embodiment, R² is preferably an optionally substituted C₁₋₆ alkyl group (e.g., a C₁₋₆ alkyl group optionally having substituent(s) selected from Substituent Group A, and a non-aromatic heterocyclic group (preferably a 3- to 14-membered non-aromatic heterocyclic group) having oxo group(s)),
(2) an optionally substituted C₃₋₁₀ cycloalkyl group (e.g., a C₃₋₁₀ cycloalkyl group optionally having substituent(s) selected from Substituent Group A), or
(3) an optionally substituted non-aromatic heterocyclic group (preferably a 3- to 14-membered non-aromatic heterocyclic group) (e.g., a non-aromatic heterocyclic group (preferably a 3- to 14-membered non-aromatic heterocyclic group) optionally having substituent(s) selected from Substituent Group A).

R² is more preferably a C₁₋₆ alkyl group (e.g., methyl, ethyl) optionally substituted by 1 to 3 substituents selected from
(i) a C₁₋₆ alkoxy-carbonyl group (e.g., methoxycarbonyl),
(ii) a C₁₋₆ alkylsulfonyl group (e.g., methylsulfonyl),
(iii) a carbamoyl group,
(iv) a cyano group,
(v) a non-aromatic heterocyclic group (preferably a 3- to 14-membered non-aromatic heterocyclic group, more preferably a 3- to 8-membered monocyclic non-aromatic heterocyclic group) (e.g., pyrrolidinyl, tetrahydrofuryl, oxetanyl) optionally substituted by 1 to 3 oxo groups, and
(vi) a halogen atom (e.g., a fluorine atom),
(2) a C₃₋₁₀ cycloalkyl group (e.g., cyclopentyl) optionally substituted by 1 to 3 hydroxy groups, or
(3) anon-aromatic heterocyclic group (preferably a 3- to 14-membered non-aromatic heterocyclic group, more preferably a 3- to 8-membered monocyclic non-aromatic heterocyclic group) (e.g., oxetanyl).

R² is furthermore preferably a C₁₋₆ alkyl group (e.g., methyl).

R³ and R⁴ are independently a hydrogen atom or a substituent, or R³ and R⁴ in combination optionally form an optionally substituted ring.

Examples of the “substituent” for R³ or R⁴ include those similar to the “substituent” exemplified in the present specification.

The “substituent” for R³ or R⁴ is preferably an optionally substituted hydrocarbon group (e.g., a hydrocarbon group optionally having substituent(s) selected from Substituent Group A), more preferably an optionally substituted C₁₋₆ alkyl group (e.g., a C₁₋₆ alkyl group optionally having substituent(s) selected from Substituent Group A).

Examples of the “ring” of the “optionally substituted ring” formed by R³ and R⁴ include a C₃₋₁₀ cycloalkane, a C₃₋₁₀ cycloalkene and a non-aromatic heterocycle (preferably a 3- to 14-membered non-aromatic heterocycle).

The “ring” of the “optionally substituted ring” formed by R³ and R⁴ optionally has 1 to 3 substituents selected from Substituent Group A at substitutable position(s). When the number of the substituents is plural, the respective substituents may be the same or different.

R³ and R⁴ are preferably independently a hydrogen atom or a substituent.

R³ and R⁴ are more preferably independently a hydrogen atom or an optionally substituted C₁₋₆ alkyl group (e.g., a C₁₋₆ alkyl group optionally having substituent(s) selected from Substituent Group A).

In one embodiment, R³ and R⁴ are furthermore preferably independently a hydrogen atom or a C₁₋₆ alkyl group (e.g., methyl).

In another embodiment, R³ and R⁴ are furthermore preferably independently
(1) a hydrogen atom, or
(2) a C₁₋₆ alkyl group (e.g., methyl) optionally substituted by 1 to 3 substituents selected from an amino group optionally mono- or di-substituted by C₁₋₆ alkyl group(s) (e.g., methyl).

Still more preferably, one of R³ and R⁴ is a hydrogen atom, and the other is
(1) a hydrogen atom, or
(2) a C₁₋₆ alkyl group (e.g., methyl) optionally substituted by 1 to 3 substituents selected from an amino group optionally mono- or di-substituted by C1-6 alkyl group(s) (e.g., methyl).

Still furthermore preferably, one of R³ and R⁴ is a hydrogen atom, and the other is a hydrogen atom or a C₁₋₆ alkyl group (e.g., methyl).

R³ and R⁴ are particularly preferably both hydrogen atoms.

R⁵ and R⁶ are independently a hydrogen atom or a substituent, or R⁵ and R⁶ in combination optionally form an optionally substituted ring.

In one embodiment, examples of the “substituent” for R⁵ or R⁶ include those similar to the “substituent” exemplified in the present specification.

The “substituent” for R⁵ or R⁶ is preferably
(1) an optionally substituted hydroxy group,
(2) an optionally substituted hydrocarbon group (e.g., a hydrocarbon group optionally having substituent(s) selected from Substituent Group A),
(3) an optionally substituted amino group, or
(4) an optionally substituted heterocyclic group (e.g., a heterocyclic group optionally having substituent(s) selected from Substituent Group A),
more preferably
(1) a hydroxy group,
(2) an optionally substituted C1-6 alkyl group (e.g., a C1-6 alkyl group optionally having substituent(s) selected from Substituent Group A),
(3) an optionally substituted C1-6 alkoxy group (e.g., a C1-6 alkoxy group optionally having substituent(s) selected from Substituent Group A),
(4) an amino group optionally mono- or di-substituted by substituent(s) selected from
   (i) an optionally substituted C₁₋₆ alkyl group (e.g., a C₁₋₆ alkyl group optionally having substituent(s) selected from Substituent Group A),
   (ii) an optionally substituted C₁₋₆ alkyl-carbonyl group (e.g., a C₁₋₆ alkyl-carbonyl group optionally having substituent(s) selected from Substituent Group A), and
   (iii) an optionally substituted C₁₋₆ alkylsulfonyl group (e.g., a C₁₋₆ alkylsulfonyl group optionally having substituent(s) selected from Substituent Group A), or
   (5) an optionally substituted non-aromatic heterocyclic group (preferably a 3- to 14-membered non-aromatic heterocyclic group) (e.g., a non-aromatic heterocyclic group (preferably a 3- to 14-membered non-aromatic heterocyclic group) optionally having substituent(s) selected from Substituent Group A).

In another embodiment, examples of the “substituent” for R⁵ or R⁶ include a halogen atom, a cyano group, a nitro group, an optionally substituted hydrocarbon group (the “hydrocarbon group” is optionally substituted by substituent(s) selected from (1) Substituent Group A, and (2) an amino group mono- or di-substituted by substituent(s) selected from (a) a C₁₋₆ alkyl group, (b) a C₃₋₁₀ cycloalkyl group optionally substituted by 1 to 3 halogen atoms, (c) a non-aromatic heterocyclic group (preferably a 3- to 14-membered non-aromatic heterocyclic group), (d) a C₁₋₆ alkylsulfonyl group, and (e) a C₃₋₁₀ cycloalkyl-carbonyl group), an optionally substituted heterocyclic group, an acyl group, an optionally substituted amino group, an optionally substituted carbamoyl group, an optionally substituted thiocarbamoyl group, an optionally substituted sulfamoyl group, an optionally substituted hydroxy group, an optionally substituted sulfanyl (SH) group, and an optionally substituted silyl group.

The “substituent” for R⁵ or R⁶ is preferably
(1) an optionally substituted hydroxy group,
(2) an optionally substituted hydrocarbon group (e.g., a hydrocarbon group optionally having substituent(s) selected from (1) Substituent Group A, and (2) an amino group mono- or di-substituted by substituent(s) selected from (a) a C1-6 alkyl group, (b) a C3-10 cycloalkyl group optionally substituted by 1 to 3 halogen atoms, (c) a non-aromatic heterocyclic group (preferably a 3- to 14-membered non-aromatic heterocyclic group), (d) a C₁₋₆ alkylsulfonyl group, and (e) a C₃₋₁₀ cycloalkyl-carbonyl group),
(3) an optionally substituted amino group,
(4), an optionally substituted heterocyclic group (e.g., a heterocyclic group optionally having substituent(s) selected from Substituent Group A), or
(5) an acyl group,
more preferably
(1) a hydroxy group,
(2) an optionally substituted C₁₋₆ alkyl group (e.g., a C₁₋₆ alkyl group optionally having substituent(s) selected from (1) Substituent Group A, and (2) an amino group mono- or di-substituted by substituent(s) selected from (a) a C₁₋₆ alkyl group, (b) a C₃₋₁₀ cycloalkyl group optionally substituted by 1 to 3 halogen atoms, (c) a non-aromatic heterocyclic group (preferably a 3- to 14-membered non-aromatic heterocyclic group), (d) a C₁₋₆ alkylsulfonyl group, and (e) a C₃₋₁₀ cycloalkyl-carbonyl group),
(3) an optionally substituted C₁₋₆ alkoxy group (e.g., a C₁₋₆ alkoxy group optionally having substituent(s) selected from Substituent Group A),
(4) an amino group optionally mono- or di-substituted by substituent(s) selected from
   (i) an optionally substituted C₁₋₆ alkyl group (e.g., a C₁₋₆ alkyl group optionally having substituent(s) selected from Substituent Group A),
   (ii) an optionally substituted C₁₋₆ alkyl-carbonyl group (e.g., a C₁₋₆ alkyl-carbonyl group optionally having substituent(s) selected from Substituent Group A), and
   (iii) an optionally substituted C₁₋₆ alkylsulfonyl group (e.g., a C₁₋₆ alkylsulfonyl group optionally having substituent(s) selected from Substituent Group A),
   (5) an optionally substituted non-aromatic heterocyclic group (preferably a 3- to 14-membered non-aromatic heterocyclic group) (e.g., a non-aromatic heterocyclic group (preferably a 3- to 14-membered non-aromatic heterocyclic group) optionally having substituent(s) selected from Substituent Group A),
   (6) a carboxy group, or
   (7) a carbamoyl group optionally mono- or di-substituted by C₁₋₆ alkyl group(s).

Examples of the “ring” of the “optionally substituted ring” formed by R⁵ and R⁶ include a C₃₋₁₀ cycloalkane, a C₃₋₁₀ cycloalkene and a non-aromatic heterocycle (preferably a 3- to 14-membered non-aromatic heterocycle), and preferable examples thereof include a C₃₋₁₀ cycloalkane and a non-aromatic heterocycle (preferably a 3- to 14-membered non-aromatic heterocycle).

The “ring” of the “optionally substituted ring” formed by R⁵ and R⁶ optionally has 1 to 3 substituents selected from Substituent Group A at substitutable position(s). When the number of the substituents is plural, the respective substituents may be the same or different.

In one embodiment, R⁵ and R⁶ are preferably independently a hydrogen atom or a substituent.

R⁵ and R⁶ are more preferably independently
(1) a hydrogen atom,
(2) a hydroxy group,
(3) an optionally substituted C1-6 alkyl group (e.g., a C1-6 alkyl group optionally having substituent(s) selected from Substituent Group A),
(4) an optionally substituted C1-6 alkoxy group (e.g., a C1-6 alkoxy group optionally having substituent(s) selected from Substituent Group A),
(5) an amino group optionally mono- or di-substituted by substituent(s) selected from
   (i) an optionally substituted C₁₋₆ alkyl group (e.g., a C₁₋₆ alkyl group optionally having substituent(s) selected from Substituent Group A),
   (ii) an optionally substituted C₁₋₆ alkyl-carbonyl group (e.g., a C₁₋₆ alkyl-carbonyl group optionally having substituent(s) selected from Substituent Group A), and
   (iii) an optionally substituted C₁₋₆ alkylsulfonyl group (e.g., a C₁₋₆ alkylsulfonyl group optionally having substituent(s) selected from Substituent Group A), or
(6) an optionally substituted non-aromatic heterocyclic group (preferably a 3- to 14-membered non-aromatic heterocyclic group) (e.g., a non-aromatic heterocyclic group (preferably a 3- to 14-membered non-aromatic heterocyclic group) optionally having substituent(s) selected from Substituent Group A).

R⁵ and R⁶ are furthermore preferably independently
(1) a hydrogen atom,
(2) a hydroxy group,
(3) a C1-6 alkyl group (e.g., methyl) optionally substituted by 1 to 3 substituents selected from
   (i) a hydroxy group, and
   (ii) an amino group optionally mono- or di-substituted by C₁₋₆ alkyl group(s) (e.g., methyl, ethyl),
   (4) a C₁₋₆ alkoxy group (e.g., methoxy),
   (5) an amino group optionally mono- or di-substituted by substituent(s) selected from
   (i) a C₁₋₆ alkyl group (e.g., methyl, ethyl),
   (ii) a C₁₋₆ alkyl-carbonyl group (e.g., acetyl), and
   (iii) a C₁₋₆ alkylsulfonyl group (e.g., methylsulfonyl), or
   (6) a non-aromatic heterocyclic group (preferably a 3- to 14-membered non-aromatic heterocyclic group, more preferably a 3- to 8-membered monocyclic non-aromatic heterocyclic group) (e.g., morpholinyl).

Still more preferably, one of R⁵ and R⁶ is a hydrogen atom, and the other is
(1) a hydrogen atom,
(2) a hydroxy group,
(3) a C1-6 alkyl group (e.g., methyl) optionally substituted by 1 to 3 substituents selected from
   (i) a hydroxy group, and
   (ii) an amino group optionally mono- or di-substituted by C₁₋₆ alkyl group(s) (e.g., methyl, ethyl),
   (4) a C₁₋₆ alkoxy group (e.g., methoxy),
   (5) an amino group optionally mono- or di-substituted by substituent(s) selected from
   (i) a C₁₋₆ alkyl group (e.g., methyl, ethyl),
   (ii) a C₁₋₆ alkyl-carbonyl group (e.g., acetyl), and
   (iii) a C₁₋₆ alkylsulfonyl group (e.g., methylsulfonyl), or
   (6) a non-aromatic heterocyclic group (preferably a 3- to 14-membered non-aromatic heterocyclic group, more preferably a 3- to 8-membered monocyclic non-aromatic heterocyclic group) (e.g., morpholinyl).

Particularly preferably, one of R⁵ and R⁶ is a hydrogen atom, and the other is
(1) a hydrogen atom,
(2) a hydroxy group,
(3) a C1-6 alkyl group (e.g., methyl) optionally substituted by amino group(s) optionally mono- or di-substituted by C1-6 alkyl group(s) (e.g., methyl), or
(4) an amino group optionally mono- or di-substituted by C1-6 alkyl group(s) (e.g., methyl).

In another embodiment, R⁵ and R⁶ are preferably independently
(1) a hydrogen atom,
(2) a hydroxy group,
(3) an optionally substituted C1-6 alkyl group (e.g., a C1-6 alkyl group optionally having substituent(s) selected from (1) Substituent Group A, and (2) an amino group mono- or di-substituted by substituent(s) selected from (a) a C1-6 alkyl group, (b) a C3-10 cycloalkyl group optionally substituted by 1 to 3 halogen atoms, (c) a non-aromatic heterocyclic group (preferably a 3- to 14-membered non-aromatic heterocyclic group), (d) a C1-6 alkylsulfonyl group, and (e) a C3-10 cycloalkyl-carbonyl group),
(4) an optionally substituted C1-6 alkoxy group (e.g., a C1-6 alkoxy group optionally having substituent(s) selected from Substituent Group A),
(5) an amino group optionally mono- or di-substituted by substituent(s) selected from
   (i) an optionally substituted C₁₋₆ alkyl group (e.g., a C₁₋₆ alkyl group optionally having substituent(s) selected from Substituent Group A),
   (ii) an optionally substituted C₁₋₆ alkyl-carbonyl group (e.g., a C₁₋₆ alkyl-carbonyl group optionally having substituent(s) selected from Substituent Group A), and
   (iii) an optionally substituted C₁₋₆ alkylsulfonyl group (e.g., a C₁₋₆ alkylsulfonyl group optionally having substituent(s) selected from Substituent Group A),
   (6) an optionally substituted non-aromatic heterocyclic group (preferably a 3- to 14-membered non-aromatic heterocyclic group) (e.g., a non-aromatic heterocyclic group (preferably a 3- to 14-membered non-aromatic heterocyclic group) optionally having substituent(s) selected from Substituent Group A),
   (7) a carboxy group, or
   (8) a carbamoyl group optionally mono- or di-substituted by C₁₋₆ alkyl group(s), or
   R⁵ and R⁶ in combination optionally form.
   (1) an optionally substituted non-aromatic heterocycle (preferably a 3- to 14-membered non-aromatic heterocycle) (e.g., a non-aromatic heterocycle (preferably a 3- to 14-membered non-aromatic heterocycle) optionally having substituent(s) selected from Substituent Group A), or
   (2) an optionally substituted C₃₋₁₀ cycloalkane (e.g., a C₃₋₁₀ cycloalkane optionally having substituent(s) selected from Substituent Group A).

R⁵ and R⁶ are more preferably independently
(1) a hydrogen atom,
(2) a hydroxy group,
(3) a C1-6 alkyl group (e.g., methyl) optionally substituted by 1 to 3 substituents selected from
   (i) a hydroxy group,
   (ii) an amino group optionally mono- or di-substituted by substituent(s) selected from
      (a) a C₁₋₆ alkyl group (e.g., methyl, ethyl) optionally substituted by 1 to 3 halogen atoms (e.g., a fluorine atom),
      (b) a C₃₋₁₃ cycloalkyl group (e.g., cyclopropyl, cyclobutyl) optionally substituted by 1 to 3 halogen atoms (e.g., a fluorine atom),
      (c) a non-aromatic heterocyclic group (preferably a 3- to 14-membered non-aromatic heterocyclic group, more preferably a 3- to 8-membered monocyclic non-aromatic heterocyclic group) (e.g., oxetanyl),
      (d) a C₁₋₆ alkylsulfonyl group (e.g., methylsulfonyl),
      (e) a C₁₋₆ alkyl-carbonyl group (e.g., acetyl), and
      (f) a C₃₋₁₀ cycloalkyl-carbonyl group (e.g., cyclopropylcarbonyl),
   (iii) a halogen atom (e.g., a fluorine atom),
   (iv) a C₁₋₆ alkylsulfanyl group (e.g., methylsulfanyl),
   (v) a C₁₋₆ alkylsulfinyl group (e.g., methylsulfinyl), and
   (vi) a C₁₋₆ alkylsulfonyl group (e.g., methylsulfonyl),
   (4) a C₁₋₆ alkoxy group (e.g., methoxy),
   (5) an amino group optionally mono- or di-substituted by substituent(s) selected from
   (i) a C₁₋₆ alkyl group (e.g., methyl, ethyl),
   (ii) a C₁₋₆ alkyl-carbonyl group (e.g., acetyl), and
   (iii) a C₁₋₆ alkylsulfonyl group (e.g., methylsulfonyl),
   (6) a non-aromatic heterocyclic group (preferably a 3- to 14-membered non-aromatic heterocyclic group, more preferably a 3- to 8-membered monocyclic non-aromatic heterocyclic group) (e.g., morpholinyl),
   (7) a carboxy group, or
   (8) a carbamoyl group optionally mono- or di-substituted by C₁₋₆ alkyl group(s) (e.g., methyl), or
   R⁵ and R⁶ in combination optionally form
   (1) a non-aromatic heterocycle (preferably a 3- to 14-membered non-aromatic heterocycle, more preferably a 3- to 8-membered monocyclic non-aromatic heterocycle) (e.g., tetrahydrofuran), or
   (2) a C₃₋₁₀ cycloalkane (e.g., cyclopentane).

Furthermore preferably, one of R⁵ and R⁶ is a hydrogen atom or a C₁₋₆ alkyl group (e.g., methyl), and the other is
(1) a hydrogen atom,
(2) a hydroxy group,
(3) a C1-6 alkyl group (e.g., methyl) optionally substituted by 1 to 3 substituents selected from
   (i) a hydroxy group,
   (ii) an amino group optionally mono- or di-substituted by substituent(s) selected from
      (a) a C₁₋₆ alkyl group (e.g., methyl, ethyl) optionally substituted by 1 to 3 halogen atoms (e.g., a fluorine atom),
      (b) a C₃₋₁₀ cycloalkyl group (e.g., cyclopropyl, cyclobutyl) optionally substituted by 1 to 3 halogen atoms (e.g., a fluorine atom),
      (c) a non-aromatic heterocyclic group (preferably a 3- to 14-membered non-aromatic heterocyclic group, more preferably a 3- to 8-membered monocyclic non-aromatic heterocyclic group) (e.g., oxetanyl),
      (d) a C₁₋₆ alkylsulfonyl group (e.g., methylsulfonyl),
      (e) a C₁₋₆ alkyl-carbonyl group (e.g., acetyl), and
      (f) a C₃₋₁₀ cycloalkyl-carbonyl group (e.g., cyclopropylcarbonyl),
   (iii) a halogen atom (e.g., a fluorine atom),
   (iv) a C₁₋₆ alkylsulfanyl group (e.g., methylsulfanyl),
   (v) a C₁₋₆ alkylsulfinyl group (e.g., methylsulfinyl), and
   (vi) a C₁₋₆ alkylsulfonyl group (e.g., methylsulfonyl),
   (4) a C₁₋₆ alkoxy group (e.g., methoxy),
   (5) an amino group optionally mono- or di-substituted by substituent(s) selected from
   (i) a C₁₋₆ alkyl group (e.g., methyl, ethyl),
   (ii) a C₁₋₆ alkyl-carbonyl group (e.g., acetyl), and
   (iii) a C₁₋₆ alkylsulfonyl group (e.g., methylsulfonyl),
   (6) a non-aromatic heterocyclic group (preferably a 3- to 14-membered non-aromatic heterocyclic group, more preferably a 3- to 8-membered monocyclic non-aromatic heterocyclic group) (e.g., morpholinyl),
   (7) a carboxy group, or
   (8) a carbamoyl group optionally mono- or di-substituted by C₁₋₆ alkyl group(s) (e.g., methyl), or
   R⁵ and R⁶ in combination optionally form
   (1) a non-aromatic heterocycle (preferably a 3- to 14-membered non-aromatic heterocycle, more preferably a 3- to 8-membered monocyclic non-aromatic heterocycle) (e.g., tetrahydrofuran), or
   (2) a C₃₋₁₀ cycloalkane (e.g., cyclopentane).

Still more preferably, one of R⁵ and R⁶ is a hydrogen atom, and the other is
(1) a hydrogen atom,
(2) a hydroxy group,
(3) a C1-6 alkyl group (e.g., methyl) optionally substituted by 1 to 3 substituents selected from
   (i) an amino group optionally mono- or di-substituted by C₁₋₆ alkyl group(s) (e.g., methyl), and
   (ii) a hydroxy group, or
   (4) an amino group optionally mono- or di-substituted by C₁₋₆ alkyl group(s) (e.g., methyl).

Particularly preferably, one of R⁵ and R⁶ is a hydrogen atom, and the other is
(1) a hydrogen atom,
(2) a C1-6 alkyl group (e.g., methyl) optionally substituted by amino group(s) optionally mono- or di-substituted by C1-6 alkyl group(s) (e.g., methyl), or
(3) an amino group optionally mono- or di-substituted by C1-6 alkyl group(s) (e.g., methyl).

Especially, R⁵ and R⁶ are particularly preferably both hydrogen atoms.

X is CR⁷R⁸, NR⁹, O or S.

X is preferably CR⁷R⁸, NR⁹ or O.

X is more preferably CR⁷R⁸ or NR⁹.

In one embodiment, X is furthermore preferably CR⁷R⁸.

In another embodiment, X is furthermore preferably NR⁹.

R⁷ and R⁸ are independently a hydrogen atom or a substituent, or R⁷ and R⁸ in combination optionally form an optionally substituted ring.

Examples of the “substituent” for R⁷ or R⁸ include those similar to the “substituent” exemplified in the present specification.

In one embodiment, the “substituent” for R⁷ or R⁸ is preferably
(1) a cyano group, or
(2) an optionally substituted hydrocarbon group (e.g., a hydrocarbon group optionally having substituent(s) selected from Substituent Group A),
more preferably
(1) a cyano group, or
(2) an optionally substituted C₁₋₆ alkyl group (e.g., a C₁₋₆ alkyl group optionally having substituent(s) selected from Substituent Group A).

In another embodiment, the “substituent” for R⁷ or R⁸ is preferably
(1) a cyano group,
(2) an optionally substituted hydrocarbon group (e.g., a hydrocarbon group optionally having substituent(s) selected from Substituent Group A), or
(3) an optionally substituted hydroxy group,
more preferably
(1) a cyano group,
(2) an optionally substituted C₁₋₆ alkyl group (e.g., a C₁₋₆ alkyl group optionally having substituent(s) selected from Substituent Group A), or
(3) a hydroxy group.

Examples of the “ring” of the “optionally substituted ring” formed by R⁷ and R⁸ include a C₃₋₁₀ cycloalkane, a C₃₋₁₀ cycloalkene and a non-aromatic heterocycle (preferably a 3- to 14-membered non-aromatic heterocycle), and preferable examples thereof include a C₃₋₁₀ cycloalkane and a non-aromatic heterocycle (preferably a 3- to 14-membered non-aromatic heterocycle).

In one embodiment, the “ring” of the “optionally substituted ring” formed by R⁷ and R⁸ optionally has 1 to 3 substituents selected from Substituent Group A at substitutable position(s). When the number of the substituents is plural, the respective substituents may be the same or different.

In another embodiment, the “ring” of the “optionally substituted ring” formed by R⁷ and R⁸ optionally has 1 to 3 substituents selected from Substituent Group A and a C₇₋₁₆ aralkyl group at substitutable position(s). When the number of the substituents is plural, the respective substituents may be the same or different.

In one embodiment, R⁷ and R⁸ are preferably independently a hydrogen atom or a substituent.

R⁷ and R⁸ are more preferably independently
(1) a hydrogen atom,
(2) a cyano group, or
(3) an optionally substituted C1-6 alkyl group (e.g., a C1-6 alkyl group optionally having substituent(s) selected from Substituent Group A).

R7 and R8 are furthermore preferably independently
(1) a hydrogen atom,
(2) a cyano group, or
(3) a C1-6 alkyl group (e.g., methyl, ethyl).

In another embodiment, R7 and R8 are preferably independently
(1) a hydrogen atom,
(2) a cyano group,
(3) an optionally substituted C1-6 alkyl group (e.g., a C1-6 alkyl group optionally having substituent(s) selected from Substituent Group A), or
(4) a hydroxy group, or

R⁷ and R⁸ in combination optionally form
(1) an optionally substituted C₃₋₁₀ cycloalkane (e.g., a C₃₋₁₀ cycloalkane optionally having substituent(s) selected from Substituent Group A), or
(2) an optionally substituted non-aromatic heterocycle (preferably a 3- to 14-membered non-aromatic heterocycle) (e.g., a non-aromatic heterocycle (preferably a 3- to 14-membered non-aromatic heterocycle) optionally having substituent(s) selected from Substituent Group A and a C₇₋₁₆ aralkyl group).

R⁷ and R⁸ are more preferably independently
(1) a hydrogen atom,
(2) a cyano group,
(3) a C₁₋₆ alkyl group (e.g., methyl, ethyl) optionally substituted by 1 to 3 hydroxy groups, or
(4) a hydroxy group, or

R⁷ and R⁸ in combination optionally form
(1) a C₃₋₁₀ cycloalkane (e.g., cyclohexane) optionally substituted by 1 to 3 substituents selected from
   (i) an oxo group, and
   (ii) a hydroxy group, or
   (2) a non-aromatic heterocycle (preferably a 3- to 14-membered non-aromatic heterocycle, more preferably a 3- to 8-membered monocyclic non-aromatic heterocycle) (e.g., pyrrolidine, piperidine) optionally substituted by 1 to 3 C₇₋₁₅ aralkyl groups (e.g., benzyl).

R⁷ and R⁸ are furthermore preferably independently
(1) a hydrogen atom, or
(2) a C₁₋₆ alkyl group (e.g., methyl).

R⁹ is a hydrogen atom or a substituent.

Examples of the “substituent” for R⁹ include those similar to the “substituent” exemplified in the present specification.

In one embodiment, the “substituent” for R⁹ is preferably an optionally substituted hydrocarbon group (e.g., a hydrocarbon group optionally having substituent(s) selected from Substituent Group A), more preferably an optionally substituted C₁₋₆ alkyl group (e.g., a C₁₋₆ alkyl group optionally having substituent(s) selected from Substituent Group A).

In another embodiment, the “substituent” for R⁹ is preferably an optionally substituted hydrocarbon group, more preferably
(1) an optionally substituted C₁₋₆ alkyl group (e.g., a C₁₋₆ alkyl group optionally having substituent(s) selected from Substituent Group A),
(2) an optionally substituted C₂₋₆ alkenyl group (e.g., a C₂₋₆ alkenyl group optionally having substituent(s) selected from Substituent Group A), or
(3) an optionally substituted C₇₋₁₆ aralkyl group (e.g., a C₇₋₁₆ aralkyl group optionally having substituent(s) selected from Substituent Group A).

In one embodiment, R⁹ is preferably an optionally substituted C₁₋₆ alkyl group (e.g., a C₁₋₆ alkyl group optionally having substituent(s) selected from Substituent Group A).

R⁹ is more preferably a C₁₋₆ alkyl group (e.g., methyl, ethyl) optionally substituted by 1 to 3 substituents selected from
(1) a hydroxy group, and
(2) a C₁₋₆ alkoxy group (e.g., methoxy) optionally substituted by 1 to 3 C₆₋₁₄ aryl groups (e.g., phenyl).

In another embodiment, R⁹ is preferably a hydrogen atom or an optionally substituted C₁₋₆ alkyl group (e.g., a C₁₋₆ alkyl group optionally having substituent(s) selected from Substituent Group A).

R⁹ is more preferably
(1) a hydrogen atom, or
(2) a C₁₋₆ alkyl group (e.g., methyl, ethyl) optionally substituted by 1 to 3 substituents selected from
   (i) a hydroxy group, and
   (ii) a C₁₋₆ alkoxy group (e.g., methoxy) optionally substituted by 1 to 3 C₆₋₁₄ aryl groups (e.g., phenyl).

In yet another embodiment, R⁹ is preferably
(1) a hydrogen atom,
(2) an optionally substituted C₁₋₆ alkyl group (e.g., a C₁₋₆ alkyl group optionally having substituent(s) selected from Substituent Group A),
(3) an optionally substituted C₂₋₆ alkenyl group (e.g., a C₂₋₆ alkenyl group optionally having substituent(s) selected from Substituent Group A), or
(4) an optionally substituted C₇₋₁₆ aralkyl group (e.g., a C₇₋₁₆ aralkyl group optionally having substituent(s) selected from Substituent Group A).

R⁹ is more preferably
(1) a hydrogen atom, or
(2) a C₁₋₆ alkyl group (e.g., methyl, ethyl, propyl, isopropyl) optionally substituted by 1 to 3 substituents selected from
   (i) a hydroxy group,
   (ii) a C₁₋₆ alkoxy group (e.g., methoxy) optionally substituted by 1 to 3 C₆₋₁₄ aryl groups (e.g., phenyl), and
   (iii) an amino group optionally mono- or di-substituted by C₁₋₆ alkyl group(s) (e.g., methyl),
   (3) a C₂₋₆ alkenyl group (e.g., allyl), or
   (4) a C₇₋₁₆ aralkyl group (e.g., benzyl) optionally substituted by 1 to 3 C₁₋₆ alkoxy groups (e.g., methoxy).

R⁹ is furthermore preferably
(1) a hydrogen atom, or
(2) a C₁₋₆ alkyl group (e.g., methyl, ethyl, propyl, preferably methyl, ethyl) optionally substituted by 1 to 3 hydroxy groups.

Preferable examples of compound (1) include the following compounds:

### [Compound A-1]

Compound (I) wherein
R¹ is an aromatic heterocyclic group (preferably a 5- to 14-membered aromatic heterocyclic group) or a C₆₋₁₄ aryl group, each of which is optionally substituted by 1 to 3 substituents selected from
   (1) an optionally substituted C₁₋₆ alkyl group (e.g., a C₁₋₆ alkyl group optionally having substituent(s) selected from Substituent Group A),
   (2) an optionally substituted C₆₋₁₄ aryl group (e.g., a C₆₋₁₄ aryl group optionally having substituent(s) selected from Substituent Group A),
   (3) an optionally substituted heterocyclic group (e.g., a heterocyclic group optionally having substituent(s) selected from Substituent Group A (the substituent is optionally further substituted by substituent(s) selected from Substituent Group A)),
   (4) a C₃₋₁₀ cycloalkylsulfonyl group,
   (5) a C₁₋₆ alkyl-carbonyl group, and
   (6) an aromatic heterocyclylsulfonyl group (preferably a 5- to 14-membered aromatic heterocyclylsulfonyl group);
R² is an optionally substituted C₁₋₆ alkyl group (e.g., a C₁₋₆ alkyl group optionally having substituent(s) selected from Substituent Group A);
R³ and R⁴ are independently a hydrogen atom or an optionally substituted C₁₋₆ alkyl group (e.g., a C₁₋₆ alkyl group optionally having substituent(s) selected from Substituent Group A);
R⁵ and R⁶ are independently
   (1) a hydrogen atom,
   (2) a hydroxy group,
   (3) an optionally substituted C₁₋₆ alkyl group (e.g., a C₁₋₆ alkyl group optionally having substituent(s) selected from Substituent Group A),
   (4) an optionally substituted C₁₋₆ alkoxy group (e.g., a C₁₋₆ alkoxy group optionally having substituent(s) selected from Substituent Group A),
   (5) an amino group optionally mono- or di-substituted by substituent(s) selected from
      (i) an optionally substituted C₁₋₆ alkyl group (e.g., a C₁₋₆ alkyl group optionally having substituent(s) selected from Substituent Group A),
      (ii) an optionally substituted C₁₋₆ alkyl-carbonyl group (e.g., a C₁₋₆ alkyl-carbonyl group optionally having substituent(s) selected from Substituent Group A), and
      (iii) an optionally substituted C₁₋₆ alkylsulfonyl group (e.g., a C₁₋₆ alkylsulfonyl group optionally having substituent(s) selected from Substituent Group A), or
   (6) an optionally substituted non-aromatic heterocyclic group (preferably a 3- to 14-membered non-aromatic heterocyclic group) (e.g., a non-aromatic heterocyclic group (preferably a 3- to 14-membered non-aromatic heterocyclic group) optionally having substituent(s) selected from Substituent Group A);
   X is CR⁷R⁸, NR⁹ or O;
R⁷ and R⁸ are independently
   (1) a hydrogen atom,
   (2) a cyano group, or
   (3) an optionally substituted C₁₋₆ alkyl group (e.g., a C₁₋₆ alkyl group optionally having substituent(s) selected from Substituent Group A); and
R⁹ is an optionally substituted C₁₋₆ alkyl group (e.g., a C₁₋₆ alkyl group optionally having substituent(s) selected from Substituent Group A).

### [Compound A-2]

Compound (I) wherein
R¹ is an aromatic heterocyclic group (preferably a 5- to 14-membered aromatic heterocyclic group) or a C₆₋₁₄ aryl group, each of which is optionally substituted by 1 to 3 substituents selected from
   (1) an optionally substituted C₁₋₆ alkyl group (e.g., a C₁₋₆ alkyl group optionally having substituent(s) selected from Substituent Group A),
   (2) an optionally substituted C₆₋₁₄ aryl group (e.g., a C₆₋₁₄ aryl group optionally having substituent(s) selected from Substituent Group A),
   (3) an optionally substituted heterocyclic group (e.g., a heterocyclic group optionally having substituent(s) selected from Substituent Group A (the substituent is optionally further substituted by substituent(s) selected from Substituent Group A)),
   (4) a C₃₋₁₀ cycloalkylsulfonyl group,
   (5) a C₁₋₆ alkyl-carbonyl group, and
   (6) an aromatic heterocyclylsulfonyl group (preferably a 5- to 14-membered aromatic heterocyclylsulfonyl group);
R² is an optionally substituted C₁₋₆ alkyl group (e.g., a C₁₋₆ alkyl group optionally having substituent(s) selected from Substituent Group A);
R³ and R⁴ are independently a hydrogen atom or an optionally substituted C₁₋₆ alkyl group (e.g., a C₁₋₆ alkyl group optionally having substituent(s) selected from Substituent Group A);
R⁵ and R⁶ are independently
   (1) a hydrogen atom,
   (2) a hydroxy group,
   (3) an optionally substituted C₁₋₆ alkyl group (e.g., a C₁₋₆ alkyl group optionally having substituent(s) selected from Substituent Group A),
   (4) an optionally substituted C₁₋₆ alkoxy group (e.g., a C₁₋₆ alkoxy group optionally having substituent(s) selected from Substituent Group A),
   (5) an amino group optionally mono- or di-substituted by substituent(s) selected from
   (i) an optionally substituted C₁₋₆ alkyl group (e.g., a C₁₋₆ alkyl group optionally having substituent(s) selected from Substituent Group A),
   (ii) an optionally substituted C₁₋₆ alkyl-carbonyl group (e.g., a C₁₋₆ alkyl-carbonyl group optionally having substituent(s) selected from Substituent Group A), and
   (iii) an optionally substituted C₁₋₆ alkylsulfonyl group (e.g., a C₁₋₆ alkylsulfonyl group optionally having substituent(s) selected from Substituent Group A), or
   (6) an optionally substituted non-aromatic heterocyclic group (preferably a 3- to 14-membered non-aromatic heterocyclic group) (e.g., a non-aromatic heterocyclic group (preferably a 3- to 14-membered non-aromatic heterocyclic group) optionally having substituent(s) selected from Substituent Group A);
   X is CR⁷R⁶, NR⁹ or O;
R⁷ and R⁸ are independently
   (1) a hydrogen atom,
   (2) a cyano group, or
   (3) an optionally substituted C₁₋₆ alkyl group (e.g., a C₁₋₆ alkyl group optionally having substituent(s) selected from Substituent Group A); and
R⁹ is a hydrogen atom or an optionally substituted C₁₋₆ alkyl group (e.g., a C₁₋₆ alkyl group optionally having substituent(s) selected from Substituent Group A).

### [Compound A-3]

Compound (I) wherein
R¹ is an aromatic heterocyclic group (preferably a 5- to 14-membered aromatic heterocyclic group) or a C₆₋₁₄ aryl group, each of which is optionally substituted by 1 to 3 substituents selected from
   (1) a halogen atom,
   (2) an optionally substituted C₁₋₆ alkyl group (e.g., a C₁₋₆ alkyl group optionally having substituent(s) selected from Substituent Group A),
   (3) an optionally substituted C₆₋₁₄ aryl group (e.g., a C6-14 aryl group optionally having substituent(s) selected from Substituent Group A),
   (4) an optionally substituted heterocyclic group (e.g., a heterocyclic group optionally having substituent(s) selected from Substituent Group A and a thioxo group (the substituent is optionally further substituted by substituent(s) selected from Substituent Group A, an azido group and a mono- or di-C₁₋₆ alkylamino group (the alkyl is substituted by substituent(s) selected from a C₃₋₁₀cycloalkyl group and a halogen atom))),
   (5) a C₃₋₁₀ cycloalkylsulfonyl group,
   (6) a C₁₋₆ alkyl-carbonyl group,
   (7) an aromatic heterocyclylsulfonyl group (preferably a 5- to 14-membered aromatic heterocyclylsulfonyl group), and
   (8) a halogenated sulfanyl group;
R² is
   (1) an optionally substituted C₁₋₆ alkyl group (e.g., a C₁₋₆ alkyl group optionally having substituent(s) selected from Substituent Group A, and a non-aromatic heterocyclic group (preferably a 3- to 14-membered non-aromatic heterocyclic group) having oxo group(s)),
   (2) an optionally substituted C₃₋₁₀ cycloalkyl group (e.g., a C₃₋₁₀ cycloalkyl group optionally having substituent(s) selected from Substituent Group A), or
   (3) an optionally substituted non-aromatic heterocyclic group (preferably a 3- to 14-membered non-aromatic heterocyclic group) (e.g., a non-aromatic heterocyclic group (preferably a 3- to 14-membered non-aromatic heterocyclic group) optionally having substituent(s) selected from Substituent Group A); R³ and R⁴ are independently a hydrogen atom or an optionally substituted C₁₋₆ alkyl group (e.g., a C₁₋₆ alkyl group optionally having substituent(s) selected from Substituent Group A);
R⁵ and R⁶ are independently
   (1) a hydrogen atom,
   (2) a hydroxy group,
   (3) an optionally substituted C₁₋₆ alkyl group (e.g., a C₁₋₆ alkyl group optionally having substituent(s) selected from (1) Substituent Group A, and (2) an amino group mono- or di-substituted by substituent(s) selected from (a) a C₁₋₅ alkyl group, (b) a C₃₋₁₀ cycloalkyl group optionally substituted by 1 to 3 halogen atoms, (c) a non-aromatic heterocyclic group (preferably a 3- to 14-membered non-aromatic heterocyclic group), (d) a C₁₋₆ alkylsulfonyl group, and (e) a C3-10 cycloalkyl-carbonyl group),
   (4) an optionally substituted C₁₋₆ alkoxy group (e.g., a C₁₋₆ alkoxy group optionally having substituent(s) selected from Substituent Group A),
   (5) an amino group optionally mono- or di-substituted by substituent(s) selected from
   (i) an optionally substituted C₁₋₆ alkyl group (e.g., a C₁₋₆ alkyl group optionally having substituent(s) selected from Substituent Group A),
   (ii) an optionally substituted C₁₋₆ alkyl-carbonyl group (e.g., a C₁₋₆ alkyl-carbonyl group optionally having substituent(s) selected from Substituent Group A), and
   (iii) an optionally substituted C₁₋₆ alkylsulfonyl group (e.g., a C₁₋₆ alkylsulfonyl group optionally having substituent(s) selected from Substituent Group A),
   (6) an optionally substituted non-aromatic heterocyclic group (preferably a 3- to 14-membered non-aromatic heterocyclic group) (e.g., a non-aromatic heterocyclic group (preferably a 3- to 14-membered non-aromatic heterocyclic group) optionally having substituent(s) selected from Substituent Group A),
   (7) a carboxy group, or
   (8) a carbamoyl group optionally mono- or di-substituted by C₁₋₆ alkyl group(s), or
R⁵ and R⁶ in combination optionally form
   (1) an optionally substituted non-aromatic heterocycle (preferably a 3- to 14-membered non-aromatic heterocycle) (e.g., a non-aromatic heterocycle (preferably a 3- to 14-membered non-aromatic heterocycle) optionally having substituent(s) selected from Substituent Group A), or
   (2) an optionally substituted C₃₋₁₀ cycloalkane (e.g., a C₃₋₁₀ cycloalkane optionally having substituent(s) selected from Substituent Group A);
   X is CR⁷R⁸, NR⁹, O or S;
R⁷ and R⁸ are independently
   (1) a hydrogen atom,
   (2) a cyano group,
   (3) an optionally substituted C₁₋₆ alkyl group (e.g., a C₁₋₆ alkyl group optionally having substituent(s) selected from Substituent Group A), or
   (4) a hydroxy group, or
R⁷ and R⁸ in combination optionally form
   (1) an optionally substituted C₃₋₁₀ cycloalkane (e.g., a C₃₋₁₀ cycloalkane optionally having substituent(s) selected from Substituent Group A), or
   (2) an optionally substituted non-aromatic heterocycle (preferably a 3- to 14-membered non-aromatic heterocycle) (e.g., a non-aromatic heterocycle (preferably a 3- to 14-membered non-aromatic heterocycle) optionally having substituent(s) selected from Substituent Group A and a C₇₋₁₆ aralkyl group); and
R⁹ is
   (1) a hydrogen atom,
   (2) an optionally substituted C₁₋₆ alkyl group (e.g., a C₁₋₆ alkyl group optionally having substituent(s) selected from Substituent Group A),
   (3) an optionally substituted C₂₋₆ alkenyl group (e.g., a C₂₋₆ alkenyl group optionally having substituent(s) selected from Substituent Group A), or
   (4) an optionally substituted C₇₋₁₆ aralkyl group (e.g., a C₇₋₁₆ aralkyl group optionally having substituent(s) selected from Substituent Group A).

### [Compound B-1]

Compound (I) wherein
R¹ is
(1) an aromatic heterocyclic group (preferably a 5- to 14-membered aromatic heterocyclic group, more preferably a 5- to 6-membered monocyclic aromatic heterocyclic group, a 8- to 14-membered fused polycyclic aromatic heterocyclic group) (e.g., oxazolyl, thiazolyl, thienyl, pyrazolyl, pyridyl, imidazopyridyl (e.g., imidazo[1,5-a]pyridyl), imidazopyridazinyl (e.g., imidazo[1,2-b]pyridazinyl), pyrazolopyrimidinyl (e.g., pyrazolo[1,5-a]pyrimidinyl)) optionally substituted by 1 to 3 substituents selected from
   (i) a C₁₋₆ alkyl group (e.g., methyl, ethyl, isopropyl) optionally substituted by 1 to 3 substituents selected from
      (a) a halogen atom (e.g., a fluorine atom), and
      (b) a hydroxy group,
   (ii) a C₆₋₁₄ aryl group (e.g., phenyl) optionally substituted by 1 to 3 halogen atoms (e.g., a fluorine atom),
   (iii) an aromatic heterocyclic group (preferably a 5- to 14-membered aromatic heterocyclic group, more preferably a 5- to 6-membered monocyclic aromatic heterocyclic group) (e.g., pyridyl, thienyl) optionally substituted by amino group(s) optionally mono- or di-substituted by C₁₋₆ alkyl group(s) (e.g., ethyl) optionally substituted by 1 to 3 halogen atoms (e.g., a fluorine atom),
   (iv) a non-aromatic heterocyclic group (preferably a 3- to 14-membered non-aromatic heterocyclic group, more preferably a 3- to 8-membered monocyclic non-aromatic heterocyclic group) (e.g., morpholinyl) optionally substituted by 1 to 3 C₁₋₆ alkyl groups (e.g., methyl),
   (v) a C3-10 cycloalkylsulfonyl group (e.g., cyclopentylsulfonyl), and
   (vi) a C₁₋₆ alkyl-carbonyl group (e.g., acetyl), or
   (2) a C₆₋₁₄ aryl group (e.g., phenyl) optionally substituted by 1 to 3 substituents selected from
   (i) a C₁₋₆ alkyl group (e.g., methyl),
   (ii) a C₃₋₁₀ cycloalkylsulfonyl group (e.g., cyclopentylsulfonyl), and
   (iii) an aromatic heterocyclylsulfonyl group (preferably a 5- to 14-membered aromatic heterocyclylsulfonyl group, more preferably a 5- to 6-membered monocyclic aromatic heterocyclylsulfonyl group) (e.g., thiazolylsulfonyl);
R² is a C₁₋₆ alkyl group (e.g., methyl);
R³ and R⁴ are independently a hydrogen atom or a C₁₋₆ alkyl group (e.g., methyl);
R⁵ and R⁶ are independently
   (1) a hydrogen atom,
   (2) a hydroxy group,
   (3) a C₁₋₆ alkyl group (e.g., methyl) optionally substituted by 1 to 3 substituents selected from
   (i) a hydroxy group, and
   (ii) an amino group optionally mono- or di-substituted by C₁₋₆ alkyl group(s) (e.g., methyl, ethyl),
   (4) a C₁₋₆ alkoxy group (e.g., methoxy),
   (5) an amino group optionally mono- or di-substituted by substituent(s) selected from
   (i) a C₁₋₆ alkyl group (e.g., methyl, ethyl),
   (ii) a C₁₋₆ alkyl-carbonyl group (e.g., acetyl), and
   (iii) a C₁₋₆ alkylsulfonyl group (e.g., methylsulfonyl), or
   (6) a non-aromatic heterocyclic group (preferably a 3- to 14-membered non-aromatic heterocyclic group, more preferably a 3- to 8-membered monocyclic non-aromatic heterocyclic group) (e.g., morpholinyl);
X is CR⁷R⁸, NR⁹ or O;
R⁷ and R⁸ are independently
   (1) a hydrogen atom,
   (2) a cyano group, or
   (3) a C₁₋₆ alkyl group (e.g., methyl, ethyl); and
R⁹ is a C₁₋₆ alkyl group (e.g., methyl, ethyl) optionally substituted by 1 to 3 substituents selected from
   (1) a hydroxy group, and
   (2) a C₁₋₆ alkoxy group (e.g., methoxy) optionally substituted by 1 to 3 C₆₋₁₄ aryl groups (e.g., phenyl).

### [Compound B-2]

Compound (I) wherein
R¹ is
(1) an aromatic heterocyclic group (preferably a 5- to 14-membered aromatic heterocyclic group, more preferably a 5- to 6-membered monocyclic aromatic heterocyclic group, a 8- to 14-membered fused polycyclic aromatic heterocyclic group) (e.g., oxazolyl, thiazolyl, thienyl, pyrazolyl, pyridyl, imidazopyridyl (e.g., imidazo[1,5-a]pyridyl), imidazopyridazinyl (e.g., imidazo[1,2-b]pyridazinyl), pyrazolopyrimidinyl (e.g., pyrazolo[1,5-a]pyrimidinyl)) optionally substituted by 1 to 3 substituents selected from
   (i) a C₁₋₆ alkyl group (e.g., methyl, ethyl, isopropyl) optionally substituted by 1 to 3 substituents selected from
      (a) a halogen atom (e.g., a fluorine atom), and
      (b) a hydroxy group,
   (ii) a C₆₋₁₄ aryl group (e.g., phenyl) optionally substituted by 1 to 3 halogen atoms (e.g., a fluorine atom),
   (iii) an aromatic heterocyclic group (preferably a 5- to 14-membered aromatic heterocyclic group, more preferably a 5- to 6-membered monocyclic aromatic heterocyclic group) (e.g., pyridyl, thienyl) optionally substituted by amino group(s) optionally mono- or di-substituted by C₁₋₆ alkyl group(s) (e.g., methyl, ethyl) optionally substituted by 1 to 3 substituents selected from
      (a) a halogen atom (e.g., a fluorine atom), and
      (b) a C₃₋₁₀ cycloalkyl group (e.g., cyclopropyl),
   (iv) a non-aromatic heterocyclic group (preferably a 3- to 14-membered non-aromatic heterocyclic group, more preferably a 3- to 8-membered monocyclic non-aromatic heterocyclic group) (e.g., morpholinyl) optionally substituted by 1 to 3 C₁₋₅ alkyl groups (e.g., methyl),
   (v) a C₃₋₁₀ cycloalkylsulfonyl group (e.g., cyclopentylsulfonyl), and
   (vi) a C₁₋₆ alkyl-carbonyl group (e.g., acetyl), or
   (2) a C₆₋₁₄ aryl group (e.g., phenyl) optionally substituted by 1 to 3 substituents selected from
   (i) a C₁₋₆ alkyl group (e.g., methyl),
   (ii) a C₃₋₁₀ cycloalkylsulfonyl group (e.g., cyclopentylsulfonyl), and
   (iii) an aromatic heterocyclylsulfonyl group (preferably a 5- to 14-membered aromatic heterocyclylsulfonyl group, more preferably a 5- to 6-membered monocyclic aromatic heterocyclylsulfonyl group) (e.g., thiazolylsulfonyl);
R² is a C₁₋₆ alkyl group (e.g., methyl);
R³ and R⁴ are independently a hydrogen atom or a C₁₋₆ alkyl group (e.g., methyl);
R⁵ and R⁶ are independently
   (1) a hydrogen atom,
   (2) a hydroxy group,
   (3) a C₁₋₆ alkyl group (e.g., methyl) optionally substituted by 1 to 3 substituents selected from
   (i) a hydroxy group, and
   (ii) an amino group optionally mono- or di-substituted by C₁₋₆ alkyl group(s) (e.g., methyl, ethyl),
   (4) a C₁₋₆ alkoxy group (e.g., methoxy),
   (5) an amino group optionally mono- or di-substituted by substituent(s) selected from
   (i) a C₁₋₆ alkyl group (e.g., methyl, ethyl),
   (ii) a C₁₋₆ alkyl-carbonyl group (e.g., acetyl), and
   (iii) a C₁₋₆ alkylsulfonyl group (e.g., methylsulfonyl), or
   (6) a non-aromatic heterocyclic group (preferably a 3- to 14-membered non-aromatic heterocyclic group, more preferably a 3- to 8-membered monocyclic non-aromatic heterocyclic group) (e.g., morpholinyl);
X is CR⁷R⁸, NR⁹ or O;
R⁷ and R⁸ are independently
   (1) a hydrogen atom,
   (2) a cyano group, or
   (3) a C₁₋₆ alkyl group (e.g., methyl, ethyl); and
R⁹ is
   (1) a hydrogen atom, or
   (2) a C₁₋₆ alkyl group (e.g., methyl, ethyl) optionally substituted by 1 to 3 substituents selected from
   (i) a hydroxy group, and
   (ii) a C₁₋₆ alkoxy group (e.g., methoxy) optionally substituted by 1 to 3 C₆₋₁₄ aryl groups (e.g., phenyl).

### [Compound B-3]

Compound (I) wherein
R¹ is an aromatic heterocyclic group (preferably a 5- to 14-membered aromatic heterocyclic group, more preferably a 5- to 6-membered monocyclic aromatic heterocyclic group, a 8- to 14-membered fused polycyclic aromatic heterocyclic group) (e.g., oxazolyl, thiazolyl, thienyl, pyrazolyl, pyridyl, imidazopyridyl (e.g., imidazo[1,5-a]pyridyl), imidazopyridazinyl (e.g., imidazo[1,2-b]pyridazinyl), pyrazolopyrimidinyl (e.g., pyrazolo[1,5-a]pyrimidinyl)), or a C₆₋₁₄ aryl group (e.g., phenyl), each of which is optionally substituted by 1 to 3 substituents selected from
   (1) a halogen atom (e.g., a fluorine atom),
   (2) a C₁₋₆ alkyl group (e.g., methyl, ethyl, isopropyl) optionally substituted by 1 to 3 substituents selected from
      (i) a halogen atom (e.g., a fluorine atom), and
      (ii) a hydroxy group,
   (3) a C₆₋₁₄ aryl group (e.g., phenyl) optionally substituted by 1 to 3 halogen atoms (e.g., a fluorine atom),
   (4) an aromatic heterocyclic group (preferably a 5- to 14-membered aromatic heterocyclic group, more preferably a 5- to 6-membered monocyclic aromatic heterocyclic group, a 8- to 14-membered fused polycyclic aromatic heterocyclic group) (e.g., pyridyl, thienyl, pyrimidinyl, imidazolyl, pyrazolyl, tetrazolyl, benzimidazolyl (e.g., 1H-benzimidazolyl), thiazolyl) optionally substituted by 1 to 3 substituents selected from
      (i) an amino group optionally mono- or di-substituted by C₁₋₆ alkyl group(s) (e.g., methyl, ethyl) optionally substituted by 1 to 3 substituents selected from
         (a) a halogen atom (e.g., a fluorine atom), and
         (b) a C3-10 cycloalkyl group (e.g., cyclopropyl),
      (ii) a halogen atom (e.g., a chlorine atom),
      (iii) a C₁₋₆ alkoxy group (e.g., methoxy),
      (iv) a cyano group,
      (v) a C₁₋₆ alkyl group (e.g., methyl, ethyl) optionally substituted by 1 to 3 substituents selected from
         (a) an azido group,
         (b) an amino group optionally mono- or di-substituted by C₁₋₆ alkyl group(s) (e.g., methyl, ethyl) optionally substituted by 1 to 3 substituents selected from a halogen atom (e.g., a fluorine atom) and a C₃₋₁₀ cycloalkyl group (e.g., cyclopropyl),
      (c) a hydroxy group, and
      (d) a halogen atom (e.g., a fluorine atom),
   (vi) a formyl group,
   (vii) a carboxy group,
   (viii) a carbamoyl group,
   (ix) a C₃₋₁₀ cycloalkyl group (e.g., cyclopropyl), and
   (x) a non-aromatic heterocyclic group (preferably a 3- to 14-membered non-aromatic heterocyclic group, more preferably a 3- to 8-membered monocyclic non-aromatic heterocyclic group) (e.g., dioxolanyl (e.g., 1,3-dioxolanyl)),
   (5) a non-aromatic heterocyclic group (preferably a 3- to 14-membered non-aromatic heterocyclic group, more preferably a 3- to 8-membered monocyclic non-aromatic heterocyclic group, a 9- to 14-membered fused polycyclic non-aromatic heterocyclic group, a 7- to 14-membered spiro heterocyclic group) (e.g., morpholinyl, dihydropyranyl (e.g., 3,6-dihydro-2H-pyranyl), tetrahydropyranyl, dihydropyridyl (e.g., 1,2-dihydropyridyl), dihydrobenzofuranyl (e.g., 2,3-dihydrobenzofuranyl), imidazolidinyl, pyrrolidinyl, dihydroisoxazolyl (e.g., 4,5-dihydroisoxazolyl), dihydropyrrolopyrazolyl (e.g., 5,6-dihydropyrrolo[3,4-c]pyrazolyl), piperazinyl, triazaspirononyl (e.g., 1,3,7-triazaspiro[4.4]nonyl), thiadiazaspirononyl (e.g., 7-thia-1,3-diazaspiro[4.4]nonyl), dioxidothiadiazaspirononyl (e.g., 7,7-dioxido-7-thia-1,3-diazaspiro[4.4]nonyl)) optionally substituted by 1 to 3 substituents selected from
      (i) a C₁₋₆ alkyl group (e.g., methyl, ethyl) optionally substituted by 1 to 3 substituents selected from
         (a) a hydroxy group,
         (b) an amino group optionally mono- or di-substituted by C₁₋₆ alkyl group(s) (e.g., methyl),
         (c) a cyano group, and
         (d) a C₆₋₁₄ aryl group (e.g., phenyl),
      (ii) an oxo group,
      (iii) a hydroxy group,
      (iv) a carbamoyl group, and
      (v) a thioxo group,
   (6) a C₃₋₁₀ cycloalkylsulfonyl group (e.g., cyclopentylsulfonyl),
   (7) a C₁₋₆ alkyl-carbonyl group (e.g., acetyl),
   (8) an aromatic heterocyclylsulfonyl group (preferably a 5- to 14-membered aromatic heterocyclylsulfonyl group, more preferably a 5- to 6-membered monocyclic aromatic heterocyclylsulfonyl group) (e.g., thiazolylsulfonyl), and
   (9) a halogenated thio group (e.g., pentafluorothio);
R² is
   (1) a C₁₋₆ alkyl group (e.g., methyl, ethyl) optionally substituted by 1 to 3 substituents selected from
   (i) a C₁₋₆ alkoxy-carbonyl group (e.g., methoxycarbonyl),
   (ii) a C₁₋₆ alkylsulfonyl group (e.g., methylsulfonyl),
   (iii) a carbamoyl group,
   (iv) a cyano group,
   (v) a non-aromatic heterocyclic group (preferably a 3- to 14-membered non-aromatic heterocyclic group, more preferably a 3- to 8-membered monocyclic non-aromatic heterocyclic group) (e.g., pyrrolidinyl, tetrahydrofuryl, oxetanyl) optionally substituted by 1 to 3 oxo groups, and
   (vi) a halogen atom (e.g., a fluorine atom),
   (2) a C₃₋₁₀ cycloalkyl group (e.g., cyclopentyl) optionally substituted by 1 to 3 hydroxy groups, or
   (3) a non-aromatic heterocyclic group (preferably a 3- to 14-membered non-aromatic heterocyclic group, more preferably a 3- to 8-membered monocyclic non-aromatic heterocyclic group) (e.g., oxetanyl);
R³ and R⁴ are independently
   (1) a hydrogen atom, or
   (2) a C₁₋₆ alkyl group (e.g., methyl) optionally substituted by 1 to 3 substituents selected from an amino group optionally mono- or di-substituted by C₁₋₆ alkyl group(s) (e.g., methyl);
R⁵ and R⁶ are independently
   (1) a hydrogen atom,
   (2) a hydroxy group,
   (3) a C₁₋₆ alkyl group (e.g., methyl) optionally substituted by 1 to 3 substituents selected from
   (i) a hydroxy group,
   (ii) an amino group optionally mono- or di-substituted by substituent(s) selected from
      (a) a C₁₋₆ alkyl group (e.g., methyl, ethyl) optionally substituted by 1 to 3 halogen atoms (e.g., a fluorine atom),
      (b) a C₃₋₁₀ cycloalkyl group (e.g., cyclopropyl, cyclobutyl) optionally substituted by 1 to 3 halogen atoms (e.g., a fluorine atom),
      (c) a non-aromatic heterocyclic group (preferably a 3- to 14-membered non-aromatic heterocyclic group, more preferably a 3- to 8-membered monocyclic non-aromatic heterocyclic group) (e.g., oxetanyl),
      (d) a C₁₋₆ alkylsulfonyl group (e.g., methylsulfonyl),
      (e) a C₁₋₆ alkyl-carbonyl group (e.g., acetyl), and
      (f) a C₃₋₁₀ cycloalkyl-carbonyl group (e.g., cyclopropylcarbonyl),
   (iii) a halogen atom (e.g., a fluorine atom),
   (iv) a C₁₋₆ alkylsulfanyl group (e.g., methylsulfanyl),
   (v) a C₁₋₆ alkylsulfinyl group (e.g., methylsulfinyl), and
   (vi) a C₁₋₆ alkylsulfonyl group (e.g., methylsulfonyl),
   (4) a C₁₋₆ alkoxy group (e.g., methoxy),
   (5) an amino group optionally mono- or di-substituted by substituent(s) selected from
   (i) a C₁₋₆ alkyl group (e.g., methyl, ethyl),
   (ii) a C₁₋₆ alkyl-carbonyl group (e.g., acetyl), and
   (iii) a C₁₋₆ alkylsulfonyl group (e.g., methylsulfonyl),
   (6) a non-aromatic heterocyclic group (preferably a 3- to 14-membered non-aromatic heterocyclic group, more preferably a 3- to 8-membered monocyclic non-aromatic heterocyclic group) (e.g., morpholinyl),
   (7) a carboxy group, or
   (8) a carbamoyl group optionally mono- or di-substituted by C₁₋₆ alkyl group(s) (e.g., methyl), or
R⁵ and R⁶ in combination optionally form
   (1) a non-aromatic heterocycle (preferably a 3- to 14-membered non-aromatic heterocycle, more preferably a 3- to 8-membered monocyclic non-aromatic heterocycle) (e.g., tetrahydrofuran), or
   (2) a C₃₋₁₀ cycloalkane (e.g., cyclopentane);
X is CR⁷R⁹, NR⁹, O or S;
R⁷ and R⁸ are independently
   (1) a hydrogen atom,
   (2) a cyano group,
   (3) a 01-6 alkyl group (e.g., methyl, ethyl) optionally substituted by 1 to 3 hydroxy groups, or
   (4) a hydroxy group, or
R⁷ and R⁸ in combination optionally form
   (1) a C₃₋₁₀ cycloalkane (e.g., cyclohexane) optionally substituted by 1 to 3 substituents selected from
   (i) an oxo group, and
   (ii) a hydroxy group, or
   (2) a non-aromatic heterocycle (preferably a 3- to 14-membered non-aromatic heterocycle, more preferably a 3- to 8-membered monocyclic non-aromatic heterocycle) (e.g., pyrrolidine, piperidine) optionally substituted by 1 to 3 C₇₋₁₆ aralkyl groups (e.g., benzyl); and
R⁹ is
   (1) a hydrogen atom, or
   (2) a C₁₋₆ alkyl group (e.g., methyl, ethyl, propyl, isopropyl) optionally substituted by 1 to 3 substituents selected from
   (i) a hydroxy group,
   (ii) a C₁₋₆ alkoxy group (e.g., methoxy) optionally substituted by 1 to 3 C₆₋₁₄ aryl groups (e.g., phenyl), and
   (iii) an amino group optionally mono- or di-substituted by C₁₋₆ alkyl group(s) (e.g., methyl),
   (3) a C₂₋₆ alkenyl group (e.g., allyl), or
   (4) a C₇₋₁₆ aralkyl group (e.g., benzyl) optionally substituted by 1 to 3 C₁₋₆ alkoxy groups (e.g., methoxy).

### [Compound B-4]

Compound (I) wherein
R¹ is
   (1) an aromatic heterocyclic group (preferably a 5- to 14 membered aromatic heterocyclic group, more preferably a 5- to 6-membered monocyclic aromatic heterocyclic group, a 8- to 14-membered fused polycyclic aromatic heterocyclic group) (e.g., oxazolyl, thiazolyl, thienyl, pyrazolyl, pyridyl, imidazopyridyl (e.g., imidazo[1,5-a]pyridyl), imidazopyridazinyl (e.g., imidazo[1,2-b]pyridazinyl), pyrazolopyrimidinyl (e.g., pyrazolo[1,5-a]pyrimidinyl)) optionally substituted by 1 to 3 substituents selected from
   (i) a C₁₋₆ alkyl group (e.g., methyl, ethyl, isopropyl) optionally substituted by 1 to 3 substituents selected from
      (a) a halogen atom (e.g., a fluorine atom), and
      (b) a hydroxy group,
   (ii) a C₆₋₁₄ aryl group (e.g., phenyl) optionally substituted by 1 to 3 halogen atoms (e.g., a fluorine atom),
   (iii) an aromatic heterocyclic group (preferably a 5- to 14-membered aromatic heterocyclic group, more preferably a 5- to 6-membered monocyclic aromatic heterocyclic group) (e.g., pyridyl, thienyl, pyrimidinyl, pyrazolyl, thiazolyl, imidazolyl) optionally substituted by 1 to 3 substituents selected from
      (a) an amino group optionally mono- or di-substituted by C₁₋₆ alkyl group(s) (e.g., methyl, ethyl) optionally substituted by 1 to 3 substituents selected from
         (I) a halogen atom (e.g., a fluorine atom), and
         (II) a C₃₋₁₀ cycloalkyl group (e.g., cyclopropyl),
      (b) a halogen atom (e.g., a chlorine atom),
      (c) a C₁₋₆ alkoxy group (e.g., methoxy),
      (d) a cyano group,
      (e) a C₁₋₆ alkyl group (e.g., methyl, ethyl) optionally substituted by 1 to 3 substituents selected from
         (I) an azido group,
         (II) an amino group optionally mono- or di-substituted by C₁₋₆ alkyl group(s) (e.g., methyl, ethyl) optionally substituted by 1 to 3 substituents selected from a halogen atom (e.g., a fluorine atom) and a C₃₋₁₀ cycloalkyl group (e.g., cyclopropyl),
         (III) a hydroxy group, and
         (IV) a halogen atom (e.g., a fluorine atom),
      (f) a formyl group,
      (g) a carboxy group,
      (h) a carbamoyl group,
      (i) a C₃₋₁₀ cycloalkyl group (e.g., cyclopropyl), and
      (j) a non-aromatic heterocyclic group (preferably a 3- to 14-membered non-aromatic heterocyclic group, more preferably a 3- to 8-membered monocyclic non-aromatic heterocyclic group) (e.g., dioxolanyl (e.g., 1,3-dioxolanyl)),
   (iv) a non-aromatic heterocyclic group (preferably a 3- to 14-membered non-aromatic heterocyclic group, more preferably a 3- to 8-membered monocyclic non-aromatic heterocyclic group, a 9- to 14-membered fused polycyclic non-aromatic heterocyclic group, a 7- to 14-membered spiro heterocyclic group) (e.g., morpholinyl, dihydropyranyl (e.g., 3,6-dihydro-2H-pyranyl), tetrahydropyranyl, dihydropyridyl (e.g., 1,2-dihydropyridyl), dihydrobenzofuranyl (e.g., 2,3-dihydrobenzofuranyl), imidazolidinyl, pyrrolidinyl, dihydroisoxazolyl (e.g., 4,5-dihydroisoxazolyl), dihydropyrrolopyrazolyl (e.g., 5,6-dihydropyrrolo[3,4-c]pyrazolyl), piperazinyl, triazaspirononyl (e.g., 1,3,7-triazaspiro[4.4]nonyl), thiadiazaspirononyl (e.g., 7-thia-1,3-diazaspiro[4.4]nonyl), dioxidothiadiazaspirononyl (e.g., 7,7-dioxido-7-thia-1,3-diazaspiro[4.4]nonyl)) optionally substituted by 1 to 3 substituents selected from
      (a) a C₁₋₆ alkyl group (e.g., methyl, ethyl) optionally substituted by 1 to 3 substituents selected from
         (I) a hydroxy group,
         (II) an amino group optionally mono- or di-substituted by C₁₋₆ alkyl group(s) (e.g., methyl),
         (III) a cyano group, and
         (IV) a C₆₋₁₄ aryl group (e.g., phenyl),
      (b) an oxo group,
      (c) a hydroxy group,
      (d) a carbamoyl group, and
      (e) a thioxo group,
   (v) a C₃₋₁₀ cycloalkylsulfonyl group (e.g., cyclopentylsulfonyl), and
   (vi) a C₁₋₆ alkyl-carbonyl group (e.g., acetyl), or
   (2) a C₆₋₁₄ aryl group (e.g., phenyl) optionally substituted by 1 to 3 substituents selected from
   (i) a halogen atom (e.g., a fluorine atom),
   (ii) a C₁₋₆ alkyl group (e.g., methyl),
   (iii) an aromatic heterocyclic group (preferably a 5- to 14-membered aromatic heterocyclic group, more preferably a 5- to 6-membered monocyclic aromatic heterocyclic group, a 8- to 14-membered fused polycyclic aromatic heterocyclic group) (e.g., imidazolyl, pyrazolyl, tetrazolyl, benzimidazolyl (e.g., 1H-benzimidazolyl)),
   (iv) a C₃₋₁₀ cycloalkylsulfonyl group (e.g., cyclopentylsulfonyl),
   (v) an aromatic heterocyclylsulfonyl group (preferably a 5- to 14-membered aromatic heterocyclylsulfonyl group, more preferably a 5- to 6-membered monocyclic aromatic heterocyclylsulfonyl group) (e.g., thiazolylsulfonyl),
   (vi) a halogenated thio group (e.g., pentafluorothio), and
   (vii) a non-aromatic heterocyclic group (preferably a 3- to 14-membered non-aromatic heterocyclic group, more preferably a 3- to 8-membered monocyclic non-aromatic heterocyclic group, a 7- to 14-membered spiro heterocyclic group) (e.g., imidazolidinyl, triazaspirononyl (e.g., 1,3,7-triazaspiro[4.4]nonyl)) optionally substituted by 1 to 3 substituents selected from
      (a) a C₁₋₆ alkyl group (e.g., methyl) optionally substituted by 1 to 3 amino groups, and
      (b) an oxo group;
R² is
   (1) a C₁₋₆ alkyl group (e.g., methyl, ethyl) optionally substituted by 1 to 3 substituents selected from
   (i) a C₁₋₆ alkoxy-carbonyl group (e.g., methoxycarbonyl),
   (ii) a C₁₋₆ alkylsulfonyl group (e.g., methylsulfonyl),
   (iii) a carbamoyl group,
   (iv) a cyano group,
   (v) a non-aromatic heterocyclic group (preferably a 3- to 14-membered non-aromatic heterocyclic group, more preferably a 3- to 8-membered monocyclic non-aromatic heterocyclic group) (e.g., pyrrolidinyl, tetrahydrofuryl, oxetanyl) optionally substituted by 1 to 3 oxo groups, and
   (vi) a halogen atom (e.g., a fluorine atom),
   (2) a C₃₋₁₀ cycloalkyl group (e.g., cyclopentyl) optionally substituted by 1 to 3 hydroxy groups, or
   (3) a non-aromatic heterocyclic group (preferably a 3- to 14-membered non-aromatic heterocyclic group, more preferably a 3- to 8-membered monocyclic non-aromatic heterocyclic group) (e.g., oxetanyl);
one of R³ and R⁴ is a hydrogen atom, and the other is
   (1) a hydrogen atom, or
   (2) a C₁₋₆ alkyl group (e.g., methyl) optionally substituted by 1 to 3 substituents selected from an amino group optionally mono- or di-substituted by C₁₋₆ alkyl group(s) (e.g., methyl);
one of R⁵ and R⁶ is a hydrogen atom or a C₁₋₆ alkyl group (e.g., methyl), and the other is
   (1) a hydrogen atom,
   (2) a hydroxy group,
   (3) a C₁₋₆ alkyl group (e.g., methyl) optionally substituted by 1 to 3 substituents selected from
   (i) a hydroxy group,
   (ii) an amino group optionally mono- or di-substituted by substituent(s) selected from
      (a) a C₁₋₆ alkyl group (e.g., methyl, ethyl) optionally substituted by 1 to 3 halogen atoms (e.g., a fluorine atom),
      (b) a C₃₋₁₀ cycloalkyl group (e.g., cyclopropyl, cyclobutyl) optionally substituted by 1 to 3 halogen atoms (e.g., a fluorine atom),
      (c) a non-aromatic heterocyclic group (preferably a 3- to 14-membered non-aromatic heterocyclic group, more preferably a 3- to 8-membered monocyclic non-aromatic heterocyclic group) (e.g., oxetanyl),
      (d) a C₁₋₆ alkylsulfonyl group (e.g., methylsulfonyl),
      (e) a C₁₋₆ alkyl-carbonyl group (e.g., acetyl), and
      (f) a C₃₋₁₀ cycloalkyl-carbonyl group (e.g., cyclopropylcarbonyl),
   (iii) a halogen atom (e.g., a fluorine atom),
   (iv) a C₁₋₆ alkylsulfanyl group (e.g., methylsulfanyl),
   (v) a C₁₋₆ alkylsulfinyl group (e.g., methylsulfinyl), and
   (vi) a C₁₋₆ alkylsulfonyl group (e.g., methylsulfonyl),
   (4) a C₁₋₆ alkoxy group (e.g., methoxy),
   (5) an amino group optionally mono- or di-substituted by substituent(s) selected from
   (i) a C₁₋₆ alkyl group (e.g., methyl, ethyl),
   (ii) a C₁₋₆ alkyl-carbonyl group (e.g., acetyl), and
   (iii) a C₁₋₆ alkylsulfonyl group (e.g., methylsulfonyl),
   (6) a non-aromatic heterocyclic group (preferably a 3- to 14-membered non-aromatic heterocyclic group, more preferably a 3- to 8-membered monocyclic non-aromatic heterocyclic group) (e.g., morpholinyl),
   (7) a carboxy group, or
   (8) a carbamoyl group optionally mono- or di-substituted by C₁₋₆ alkyl group(s) (e.g., methyl), or
R⁵ and R⁶ in combination optionally form
   (1) a non-aromatic heterocycle (preferably a 3- to 14-membered non-aromatic heterocycle, more preferably a 3- to 8-membered monocyclic non-aromatic heterocycle) (e.g., tetrahydrofuran), or
   (2) a C₃₋₁₀ cycloalkane (e.g., cyclopentane);
X is CR⁷R⁸, NR⁹, O or S;
R⁷ and R⁸ are independently
   (1) a hydrogen atom,
   (2) a cyano group,
   (3) a C₁₋₆ alkyl group (e.g., methyl, ethyl) optionally substituted by 1 to 3 hydroxy groups, or
   (4) a hydroxy group, or
R⁷ and R⁸ in combination optionally form
   (1) a C₃₋₁₀ cycloalkane (e.g., cyclohexane) optionally substituted by 1 to 3 substituents selected from
   (i) an oxo group, and
   (ii) a hydroxy group, or
   (2) a non-aromatic heterocycle (preferably a 3- to 14-membered non-aromatic heterocycle, more preferably a 3- to 8-membered monocyclic non-aromatic heterocycle) (e.g., pyrrolidine, piperidine) optionally substituted by 1 to 3 C₇₋₁₆ aralkyl groups (e.g., benzyl); and
R⁹ is
   (1) a hydrogen atom, or
   (2) a C₁₋₆ alkyl group (e.g., methyl, ethyl, propyl, isopropyl) optionally substituted by 1 to 3 substituents selected from
   (i) a hydroxy group,
   (ii) a C₁₋₆ alkoxy group (e.g., methoxy) optionally substituted by 1 to 3 C₆₋₁₄ aryl groups (e.g., phenyl), and
   (iii) an amino group optionally mono- or di-substituted by C₁₋₆ alkyl group(s) (e.g., methyl),
   (3) a C₂₋₆ alkenyl group (e.g., allyl), or
   (4) a C₇₋₁₆ aralkyl group (e.g., benzyl) optionally substituted by 1 to 3 C₁₋₆ alkoxy groups (e.g., methoxy).

### [Compound C-1]

Compound (I) wherein
R¹ is
   (1) an aromatic heterocyclic group (preferably a 5- to 14-membered aromatic heterocyclic group, more preferably a 5- to 6-membered monocyclic aromatic heterocyclic group) (e.g., oxazolyl, pyridyl, pyrazolyl) optionally substituted by 1 to 3 substituents selected from
   (i) a C1-6 alkyl group (e.g., methyl) optionally substituted by 1 to 3 halogen atoms (e.g., a fluorine atom),
   (ii) an aromatic heterocyclic group (preferably a 5- to 14-membered aromatic heterocyclic group, more preferably a 5- to 6-membered monocyclic aromatic heterocyclic group) (e.g., pyridyl, pyrazolyl) optionally substituted by 1 to 3 substituents selected from
      (a) an amino group optionally mono- or di-substituted by C₁₋₆ alkyl group(s) (e.g., methyl, ethyl) optionally substituted by 1 to 3 substituents selected from
         (I) a halogen atom (e.g., a fluorine atom), and
         (II) a C₃₋₁₀ cycloalkyl group (e.g., cyclopropyl), and
      (b) a C₁₋₆ alkyl group (e.g., methyl) optionally substituted by 1 to 3 amino groups, and
   (iii) a non-aromatic heterocyclic group (preferably a 3- to 14-membered non-aromatic heterocyclic group, more preferably a 3- to 8-membered monocyclic non-aromatic heterocyclic group, a 7- to 14-membered spiro heterocyclic group) (e.g., imidazolidinyl, triazaspirononyl (e.g., 1,3,7-triazaspiro[4.4]nonyl)) optionally substituted by 1 to 3 substituents selected from
      (a) a C₁₋₆ alkyl group (e.g., methyl) optionally substituted by 1 to 3 amino groups, and
      (b) an oxo group, or
      (2) a C₆₋₁₄ aryl group (e.g., phenyl) optionally substituted by 1 to 3 C₁₋₆ alkyl groups (e.g., methyl);
R² is a C₁₋₆ alkyl group (e.g., methyl);
one of R³ and R⁴ is a hydrogen atom, and the other is a hydrogen atom or a C₁₋₆ alkyl group (e.g., methyl);
one of R⁵ and R⁶ is a hydrogen atom, and the other is
   (1) a hydrogen atom,
   (2) a hydroxy group,
   (3) a C₁₋₆ alkyl group (e.g., methyl) optionally substituted by 1 to 3 substituents selected from
   (i) an amino group optionally mono- or di-substituted by C₁₋₆ alkyl group(s) (e.g., methyl), and
   (ii) a hydroxy group, or
   (4) an amino group optionally mono- or di-substituted by C₁₋₆ alkyl group(s) (e.g., methyl);
X is CR⁷R⁹, NR⁹ or O;
R⁷ and R⁸ are independently
   (1) a hydrogen atom, or
   (2) a C₁₋₆ alkyl group (e.g., methyl); and
R⁹ is
   (1) a hydrogen atom, or
   (2) a C₁₋₆ alkyl group (e.g., methyl, ethyl, propyl) optionally substituted by 1 to 3 hydroxy groups.

### [Compound D-1]

Compound (I) wherein
R¹ is an aromatic heterocyclic group (preferably a 5- to 14-membered aromatic heterocyclic group, more preferably a 5- to 6-membered monocyclic aromatic heterocyclic group) (e.g., oxazolyl) optionally substituted by aromatic heterocyclic group(s) (preferably a 5- to 14-membered aromatic heterocyclic group, more preferably a 5- to 6-membered monocyclic aromatic heterocyclic group) (e.g., pyridyl) optionally substituted by amino group(s) optionally mono- or di-substituted by C₁₋₆ alkyl group(s) (e.g., ethyl) optionally substituted by 1 to 3 halogen atoms (e.g., a fluorine atom);
R² is a C₁₋₆ alkyl group (e.g., methyl);
R³ and R⁴ are both hydrogen atoms;
one of R⁵ and R⁶ is a hydrogen atom, and the other is
   (1) a hydrogen atom,
   (2) a hydroxy group,
   (3) a C₁₋₆ alkyl group (e.g., methyl) optionally substituted by amino group(s) optionally mono- or di-substituted by C₁₋₆ alkyl group(s) (e.g., methyl), or
   (4) an amino group optionally mono- or di-substituted by C₁₋₆ alkyl group(s) (e.g., methyl);
X is CR⁷R⁸; and
R⁷ and R⁸ are independently
   (1) a hydrogen atom, or
   (2) a C₁₋₆ alkyl group (e.g., methyl).

### [Compound D-2]

Compound (I) wherein
R¹ is an aromatic heterocyclic group (preferably a 5- to 14-membered aromatic heterocyclic group, more preferably a 5- to 6-membered monocyclic aromatic heterocyclic group) (e.g., oxazolyl) optionally substituted by aromatic heterocyclic group(s) (preferably a 5- to 14-membered aromatic heterocyclic group, more preferably a 5- to 6-membered monocyclic aromatic heterocyclic group) (e.g., pyridyl) optionally substituted by amino group(s) optionally mono- or di-substituted by C₁₋₆ alkyl group(s) (e.g., methyl, ethyl) optionally substituted by 1 to 3 substituents selected from
   (1) a halogen atom (e.g., a fluorine atom), and
   (2) a C₃₋₁₀ cycloalkyl group (e.g., cyclopropyl);
R² is a C₁₋₆ alkyl group (e.g., methyl);
R³ and R⁴ are both hydrogen atoms;
one of R⁵ and R⁶ is a hydrogen atom, and the other is
   (1) a hydrogen atom,
   (2) a C₁₋₆ alkyl group (e.g., methyl) optionally substituted by amino group(s) optionally mono- or di-substituted by C₁₋₆ alkyl group(s) (e.g., methyl), or
   (3) an amino group optionally mono- or di-substituted by C₁₋₆ alkyl group(s) (e.g., methyl);
X is CR⁷R⁸ or NR⁹;
R⁷ and R⁸ are independently
   (1) a hydrogen atom, or
   (2) a C₁₋₆ alkyl group (e.g., methyl); and
R⁹ is
   (1) a hydrogen atom, or
   (2) a C₁₋₆ alkyl group (e.g., methyl, ethyl) optionally substituted by 1 to 3 hydroxy groups.

### [Compound E-1]

Compound (I) wherein
R^{l} is an aromatic heterocyclic group (preferably a 5- to 14-membered aromatic heterocyclic group, more preferably a 5- to 6-membered monocyclic aromatic heterocyclic group) (e.g., oxazolyl) optionally substituted by aromatic heterocyclic group(s) (preferably a 5- to 14-membered aromatic heterocyclic group, more preferably a 5- to 6-membered monocyclic aromatic heterocyclic group) (e.g., pyridyl) optionally substituted by amino group(s) optionally mono- or di-substituted by C₁₋₆ alkyl group(s) (e.g., methyl, ethyl) optionally substituted by 1 to 3 halogen atoms (e.g., a fluorine atom);
R² is a C₁₋₆ alkyl group (e.g., methyl);
R³ and R⁴ are both hydrogen atoms;
R⁵ and R⁶ are both hydrogen atoms;
X is CR⁷R⁸ or NR⁹;
R⁷ and R⁸ are independently
   (1) a hydrogen atom, or
   (2) a C₁₋₆ alkyl group (e.g., methyl); and
R⁹ is
   (1) a hydrogen atom, or
   (2) a C₁₋₆ alkyl group (e.g., methyl, ethyl) optionally substituted by 1 to 3 hydroxy groups.

### [Compound F-1]

Compound (I) wherein
R¹ is an aromatic heterocyclic group (preferably a 5- to 14-membered aromatic heterocyclic group, more preferably a 5- to 6-membered monocyclic aromatic heterocyclic group) (e.g., oxazolyl) optionally substituted by aromatic heterocyclic group(s) (preferably a 5- to 14-membered aromatic heterocyclic group, more preferably a 5- to 6-membered monocyclic aromatic heterocyclic group) (e.g., pyridyl) optionally substituted by amino group(s) optionally mono- or di-substituted by C₁₋₆ alkyl group(s) (e.g., methyl, ethyl) optionally substituted by 1 to 3 halogen atoms (e.g., a fluorine atom);
R² is a C₁₋₆ alkyl group (e.g., methyl);
R³ and R⁴ are both hydrogen atoms;
R⁵ and R⁶ are both hydrogen atoms;
X is NR⁹; and
R⁹ is
   (1) a hydrogen atom, or
   (2) a C₁₋₆ alkyl group (e.g., methyl, ethyl) optionally substituted by 1 to 3 hydroxy groups.

### [Compound G-1]

N-(3-(3-(2-hydroxyethyl)-2-oxoimidazolidin-1-yl)-1-methyl-1H-pyrazol-4-yl)-2-(2-((2,2,2-trifluoroethyl)amino)pyridin-4-yl)-1,3-oxazole-4-carboxamide or a salt thereof; or
N-(1-methyl-3-(2-oxoimidazolidin-1-yl)-1H-pyrazol-4-yl)-2-(2-((2,2,2-trifluoroethyl)amino)pyridin-4-yl)-1,3-oxazole-4-carboxamide or a salt thereof.

### [Compound H-1]

N-(1-methyl-3-((3S)-3-methyl-2-oxopyrrolidin-1-yl)-1H-pyrazol-4-yl)-2-(2-((2,2,2-trifluoroethyl)amino)pyridin-4-yl)-1,3-oxazole-4-carboxamide or a salt thereof.

In one embodiment, the present disclosure provides a topical composition comprising a compound [Compound 2] of Formula (III) having the following structure:

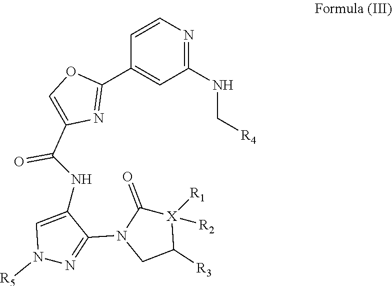

or a stereoisomer, solvates, tautomers, or pharmaceutically acceptable salts thereof, wherein:

R₁ and R₂ are independently selected from H, C₁₋₆ alkyl optionally substituted with hydroxyl;

R₃ is H, OH, —O—C₁₋₃ alkyl, or CH₂NR₆R₇;

R₄ is an optionally halogenated C₁₋₆ alkyl group (e.g., CF₃), an optionally halogenated C₃₋₁₀ cycloalkyl group, an optionally substituted non-aromatic heterocyclic group, an optionally substituted aromatic heterocyclic group, or an optionally substituted C₆₋₁₄ aryl group;

R₅ is C₁₋₆ alkyl, an optionally halo-substituted C₃₋₁₀ cycloalkyl group, or a non-aromatic heterocyclic group;

R₆ and R₇ are independently selected from H, C₁₋₆ alkyl and a C₃₋₁₀ cycloalkyl group; X is N or C; and

n is 0, 1 or 2,

wherein when X is N, then R₂ is not present.

The disclosure further provides a compound of Formula III as follows:
2.1 Compound 2, wherein Xis C and at least one of R₁ and R₂ is C₁₋₃ alkyl.
2.2 Compound 2, wherein X is C and at least one of R₁ and R₂ is H.
2.3 Compound 2, wherein X is C and R₁ is H and R₂ is C₁₋₃ alkyl.
2.4 Compound 2, wherein X is C and R₁ and R₂ are both C₁₋₃ alkyl.
2.5 Compound 2, wherein X is C and R₁ and R₂ are both H.
2.6 Compound 2, wherein X is N.
2.7 Compound 2.6, wherein R₁ is H.
2.8 Compound 2.6, wherein Ri is C₁₋₃ alkyl.
2.9 Compound 2.6 or 2.8, wherein R₁ is methyl or propyl.
2.10 Compound 2.6, wherein R₁ is C₁₋₆ alkyl substituted with hydroxyl.
2.11 Compound 2.6 or 2.10, wherein R₁ is hydroxyethyl or hydroxypropyl (e.g., 2-hydroxyethyl or 2-hydroxypropyl).
2.12 Any of compounds 2 or 2.1-2.11, wherein R₃ is H.
2.13 Any of compounds 2 or 2.1-2.11, wherein R₃ is OH.
2.14 Any of compounds 2 or 2.1-2.11, wherein R₃ is CH₂NR₆R₇ and at least one of R₆ and R₇ are H.
2.15 Any of compounds 2 or 2.1-2.11, wherein R₃ is CH₂NR₆R₇ and at least one of R₆ and R₇ are C₁₋₆ alkyl.
2.16 Any of compounds 2 or 2.1-2.11, wherein R₃ is CH₂NR₆R₇ and at least one of R₆ and R₇ are a C₃₋₁₀ cycloalkyl group (e.g., cyclopropyl).
2.17 Any of compounds 2 or 2.1-2.11, wherein R₃ is CH₂NR₆R₇ and R₆ is H and R₇ is C₁₋₆ alkyl (e.g., methyl).
2.18 Any of compounds 2 or 2.1-2.11, wherein R₃ is CH₂NR₆R₇ and both R₆ and R₇ are C₁₋₆ alkyl (e.g., methyl).
2.19 Any of compounds 2 or 2.1-2.18, wherein R₄ is a halogenated C₁₋₆ alkyl group.
2.20 Any of compounds 2 or 2.1-2.19, wherein R₄ is CF₃.
2.21 Any of compounds 2 or 2.1-2.18, wherein R₄ is an optionally halogenated C₃₋₁₀cycloalkyl group (e.g., cyclopropyl).
2.22 Any of compounds 2 or 2.1-2.21, wherein R₅ is C₁₋₆ alkyl.
2.23 Any of the preceding compounds, wherein R₅ is methyl.
2.24 Any of compounds 2 or 2.1-2.21, wherein R₅ is a non-aromatic heterocyclic group.
2.25 Any of compounds 2 or 2.1-2.21 or 2.24, wherein R₅ is a 3- to 14-membered non-aromatic heterocyclic group.
2.26 Any of compounds 2 or 2.1-2.21 or 2.24-2.25, wherein R₅ is a 4-membered non-aromatic heterocyclic group.
2.27 Any of compounds 2 or 2.1-2.21 or 2.24-2.26, wherein R₅ is oxetanyl.
2.28 Any of the preceding compounds, wherein the compound of Formula III has one of the following structures:

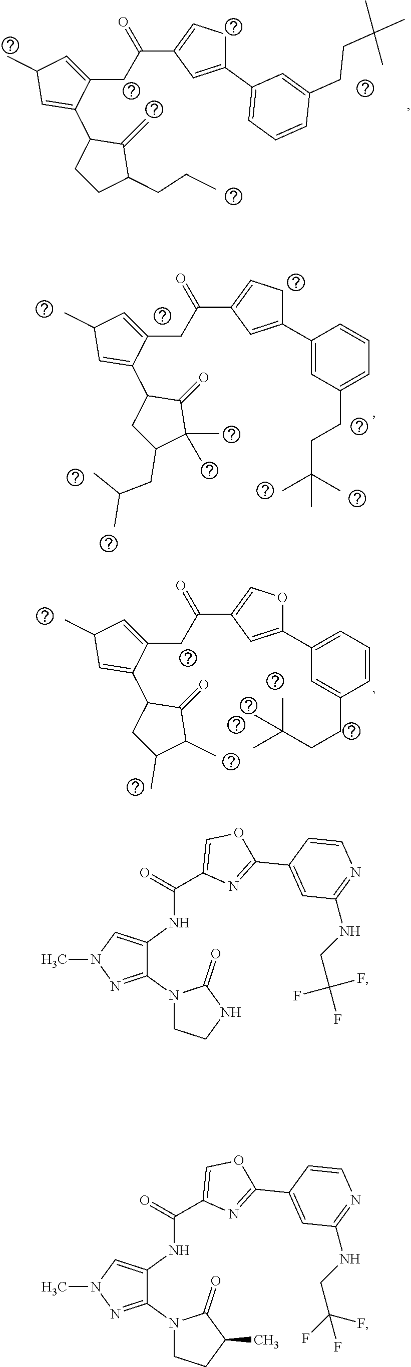

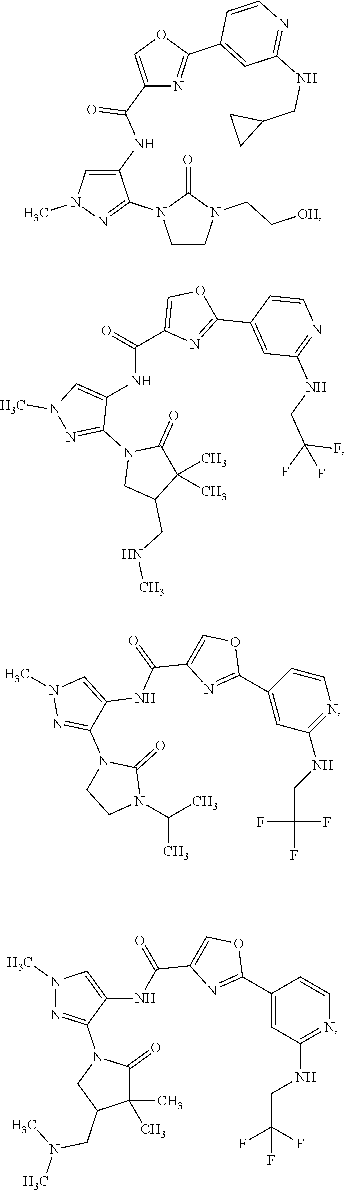

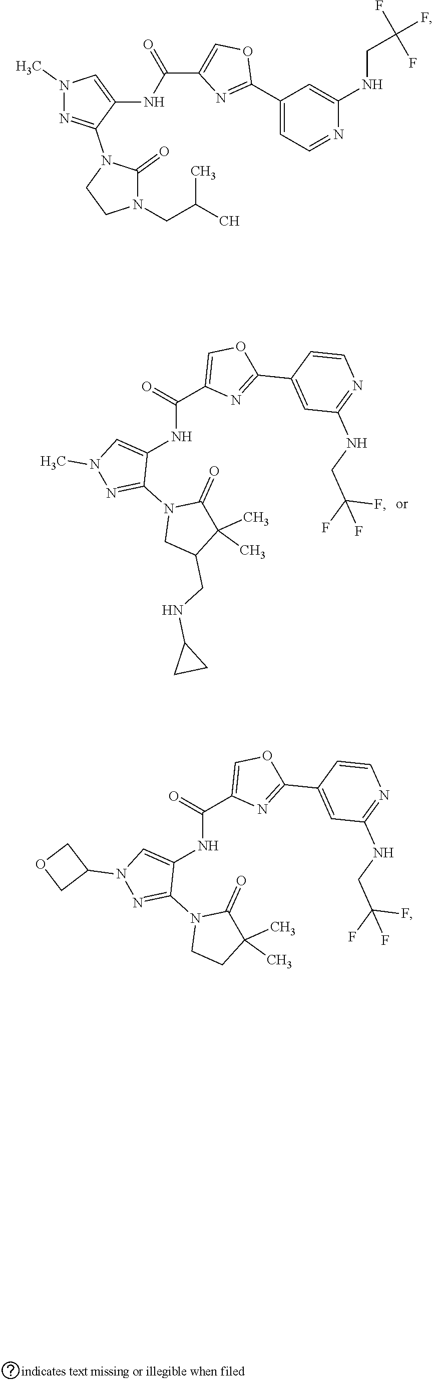 [SMILES: CC(C)CN1CCN(c2nn(C)cc2NC(=O)c2coc(-c3ccnc(NCC(F)(F)F)c3)n2)C1=O ; Cn1cc(NC(=O)c2coc(-c3ccnc(NCC(F)(F)F)c3)n2)c(N2CC(CNC3CC3)C(C)(C)C2=O)n1 ; CC1(C)CCN(c2nn(C3COC3)cc2NC(=O)c2coc(-c3ccnc(NCC(F)(F)F)c3)n2)C1=O]

or a stereoisomer, solvates, tautomers, or pharmaceutically acceptable salts thereof.
2.29 Any of the preceding compounds, wherein the compound of Formula III is selected from the following:
   N-(3-(3-(2-hydroxyethyl)-2-oxoimidazolidin-1-yl)-1-methyl-1H-pyrazol-4-yl)-2-(2-((2,2,2-trifluoroethyl)amino)pyridin-4-yl)-1,3-oxazole-4-carboxamide,
   2-(2-((2,2-difluoroethyl)amino)pyridin-4-yl)-N-(3-(3-(2-hydroxyethyl)-2-oxoimidazolidin-1-yl)-1-methyl-1H-pyrazol-4-yl)-1,3-oxazole-4-carboxamide,
   N-(3-((3S,4S)-4-hydroxy-3-methyl-2-oxopyrrolidin-1-yl)-1-methyl-1H-pyrazol-4-yl)-2-(2-((2,2,2-trifluoroethyl)amino)pyridin-4-yl)-1,3-oxazole-4-carboxamide,
   N-(1-methyl-3-(2-oxoimidazolidin-1-yl)-1H-pyrazol-4-yl)-2-(2-((2,2,2-trifluoroethyl)amino)pyridin-4-yl)-1,3-oxazole-4-carboxamide,
   N-(1-methyl-3-((3S)-3-methyl-2-oxopyrrolidin-1-yl)-1H-pyrazol-4-yl)-2-(2-((2,2,2-trifluoroethyl)amino)pyridin-4-yl)-1,3-oxazole-4-carboxamide,
   2-(2-((cyclopropylmethyl)amino)pyridin-4-yl)-N-(3-(3-(2-hydroxyethyl)-2-oxoimidazolidin-1-yl)-1-methyl-1H-pyrazol-4-yl)-1,3-oxazole-4-carboxamide,
   N-(3-(3,3-dimethyl-4-((methylamino)nethyl)-2-oxopyrrolidin-1-yl) methyl-1H-pyrazol-4-yl)-2-(2-((2,2,2-trifluoroethyl)amino)pyridin-4-yl)-1,3-oxazole-4-carboxamide,
   N-(3-(3,3-dimethyl-4-((methylamino)nethyl)-2-oxopyrrolidin-1-yl)-1-methyl-1H-pyrazol-4-yl)-2-(2-((2,2,2-trifluoroethyl)amino)pyridin-4-yl)-1,3-oxazole-4-carboxamide,
   N-(3-(3-isopropyl-2-oxoimidazolidin-1-yl)-1-methyl-1H-pyrazol-4-yl)-2-(2-((2,2,2-trifluoroethyllamino)pyridin-4-yl)-1,3-oxazole-4-carboxamide,
   N-(3-(4-((dimethylamino)nethyl)-3,3-dimethyl-2-oxopyrrolidin-1-yl)-1-methyl-1H-pyrazol-4-yl)-2-(2-((2,2,2-trifluoroethyl)amino)pyridin-4-yl)-1,3-oxazole-4-carboxamide,
   N-(3-(3-(2-hydroxypropyl)-2-oxoimidazolidin-1-yl)-1-methyl-1H-pyrazol-4-yl)-2-(2-((2,2,2-trifluoroethyl)amino)pyridin-4-yl)-1,3-oxazole-4-carboxamide,
   N-(3-(4-((dimethylamino)methyl)-3,3-dimethyl-2-oxopyrrolidin-1-yl)-1-methyl-1H-pyrazol-4-yl)-2-(2-((2,2,2-trifluoroethyl)amino)pyridin-4-yl)-1,3-oxazole-4-carboxamide,
   N-(3-(4-((cyclopropylamino)methyl)-3,3-dimethyl-2-oxopyrrolidin-1-yl)-1-methyl-1H-pyrazol-4-yl)-2-(2-((2,2,2-trifluoroethyl)amino)pyridin-4-yl)-1,3-oxazole-4-carboxamide, and
   N-(3-(3,3-dimethyl-2-oxopyrrolidin-1-yl)-1-(oxetan-3-yl)-1H-pyrazol-4-yl)-2-(2-((2,2,2-trifluoroethyl)amino)pyridin-4-yl)-1,3-oxazole-4-carboxamide,
   or a stereoisomer, solvates, tautomers, or pharmaceutically acceptable salts thereof
2.30 Compound 2.28, wherein the compounds of Formula III has the following structure:

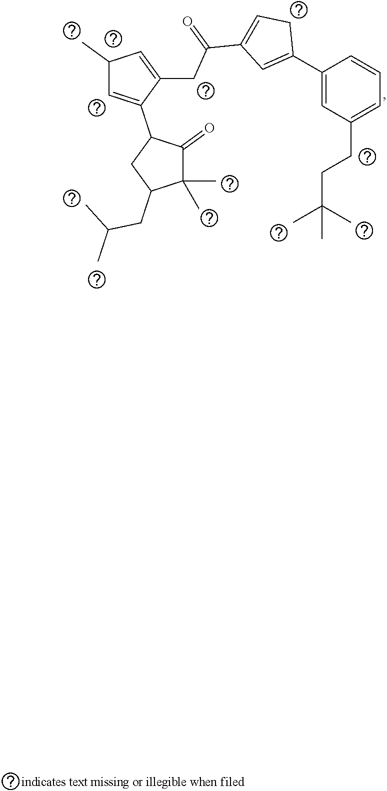 [SMILES: CC1C=C(CC(=O)C2=CCC(c3cccc(CCC(C)(C)C)c3)=C2)C(C2CC(CC(C)C)C(C)(C)C2=O)=C1]

or a stereoisomer, solvates, tautomers, or pharmaceutically acceptable salts thereof.
2.31 Compound 2.28, wherein the compounds of Formula III has the following structure:

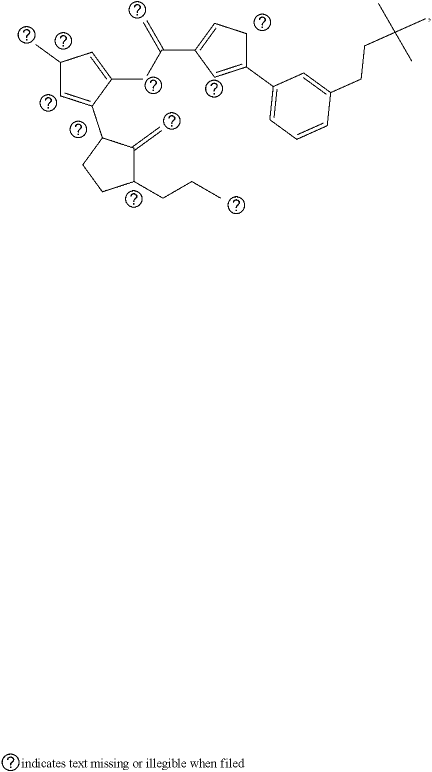 [SMILES: C=C(CC1=CC(C)C=C1C1CCC(CCC)C1=C)C1=CCC(c2cccc(CCC(C)(C)C)c2)=C1]

or a stereoisomer, solvates, tautomers, or pharmaceutically acceptable salts thereof.
2.32 Compound 2.28, wherein the compounds of Formula III has the following structure:

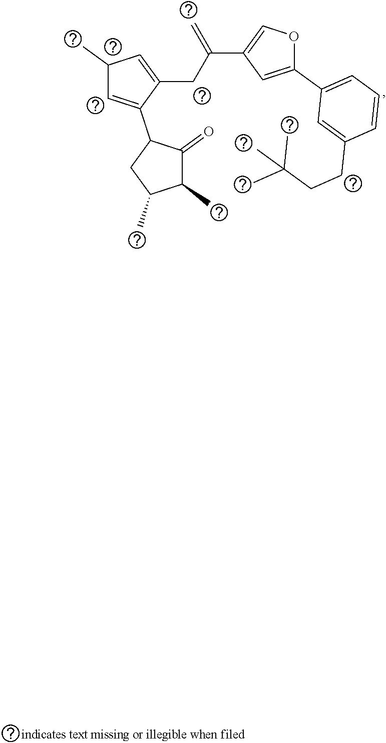 [SMILES: C=C(CC1=CC(C)C=C1C1C[C@@H](C)[C@H](C)C1=O)c1coc(-c2cccc(CCC(C)(C)C)c2)c1]

or a stereoisomer, solvates, tautomers, or pharmaceutically acceptable salts thereof.
2.33 Compound 2.28, wherein the compounds of Formula III has the following structure:

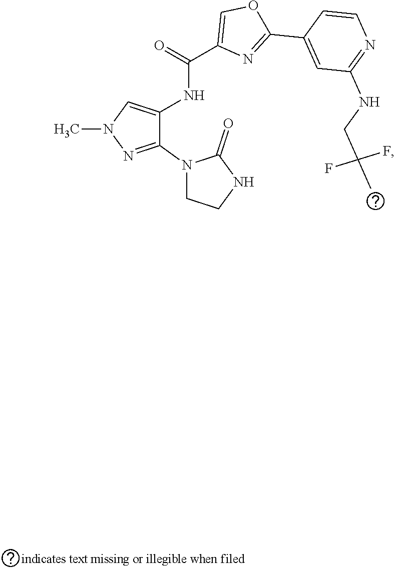 [SMILES: Cn1cc(NC(=O)c2coc(-c3ccnc(NCC(C)(F)F)c3)n2)c(N2CCNC2=O)n1]

or a stereoisomer, solvates, tautomers, or pharmaceutically acceptable salts thereof.
2.34 Compound 2.28, wherein the compounds of Formula III has the following structure:

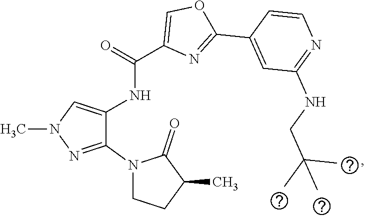 [SMILES: C[C@H]1CCN(c2nn(C)cc2NC(=O)c2coc(-c3ccnc(NCC(C)(C)C)c3)n2)C1=O]

or a stereoisomer, solvates, tautomers, or pharmaceutically acceptable salts thereof.
2.35 Compound 2.28, wherein the compounds of Formula III has the following structure:

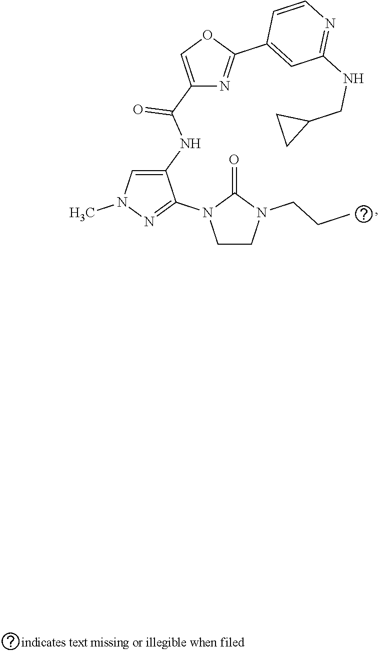 [SMILES: CCCN1CCN(c2nn(C)cc2NC(=O)c2coc(-c3ccnc(NCC4CC4)c3)n2)C1=O]

or a stereoisomer, solvates, tautomers, or pharmaceutically acceptable salts thereof.
2.36 Compound 2.28, wherein the compounds of Formula III has the following structure:

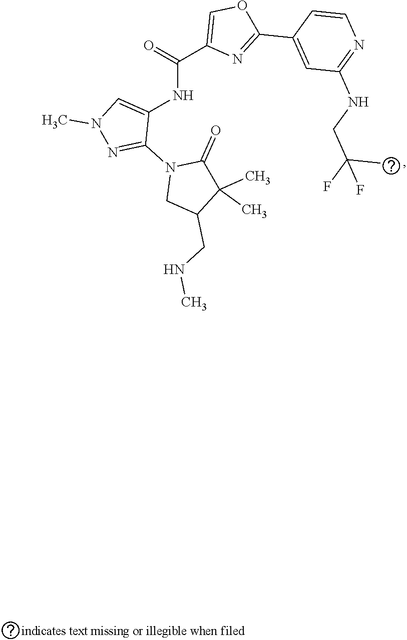 [SMILES: CNCC1CN(c2nn(C)cc2NC(=O)c2coc(-c3ccnc(NCC(C)(F)F)c3)n2)C(=O)C1(C)C]

or a stereoisomer, solvates, tautomers, or pharmaceutically acceptable salts thereof.
2.37 Compound 2.28, wherein the compounds of Formula III has the following structure:

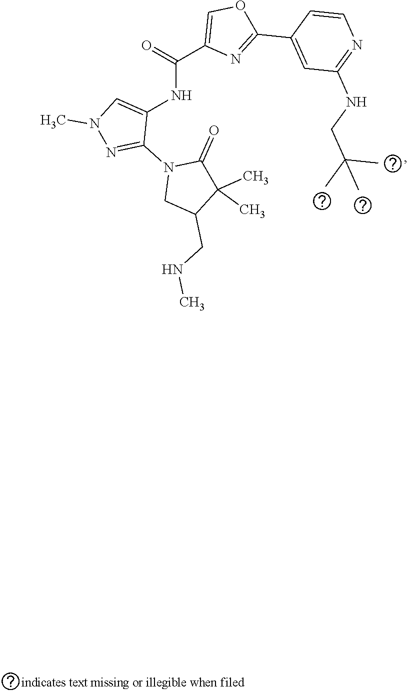 [SMILES: CNCC1CN(c2nn(C)cc2NC(=O)c2coc(-c3ccnc(NCC(C)(C)C)c3)n2)C(=O)C1(C)C]

or a stereoisomer, solvates, tautomers, or pharmaceutically acceptable salts thereof.
2.38 Compound 2.28, wherein the compounds of Formula III has the following structure:

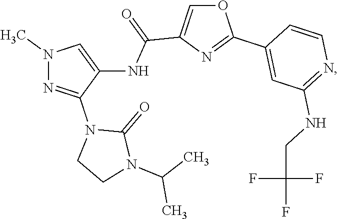 [SMILES: CC(C)N1CCN(c2nn(C)cc2NC(=O)c2coc(-c3ccnc(NCC(F)(F)F)c3)n2)C1=O]

or a stereoisomer, solvates, tautomers, or pharmaceutically acceptable salts thereof.
2.39 Compound 2.28, wherein the compounds of Formula III has the following structure:

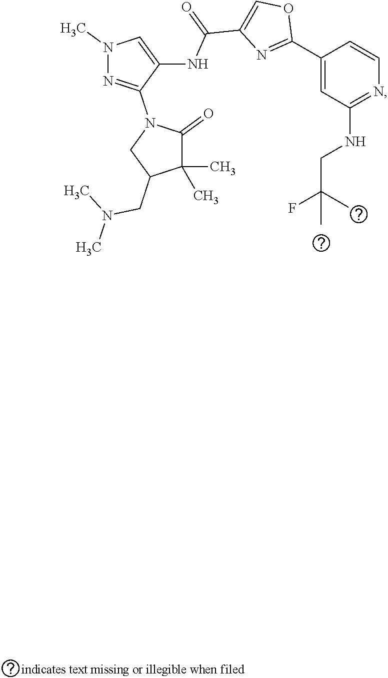 [SMILES: CN(C)CC1CN(c2nn(C)cc2NC(=O)c2coc(-c3ccnc(NCC(C)(C)F)c3)n2)C(=O)C1(C)C]

or a stereoisomer, solvates, tautomers, or pharmaceutically acceptable salts thereof.
2.40 Compound 2.28, wherein the compounds of Formula III has the following structure:

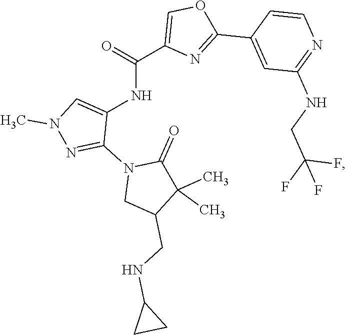 [SMILES: Cn1cc(NC(=O)c2coc(-c3ccnc(NCC(F)(F)F)c3)n2)c(N2CC(CNC3CC3)C(C)(C)C2=O)n1]

or a stereoisomer, solvates, tautomers, or pharmaceutically acceptable salts thereof.
2.41 Compound 2.28, wherein the compounds of Formula III has the following structure:

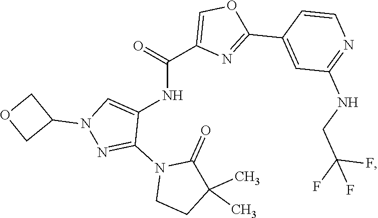 [SMILES: CC1(C)CCN(c2nn(C3COC3)cc2NC(=O)c2coc(-c3ccnc(NCC(F)(F)F)c3)n2)C1=O]

or a stereoisomer, solvates, tautomers, or pharmaceutically acceptable salts thereof.
2.42 Any of the preceding compounds, wherein the compound of Formula III has one of the following structures:

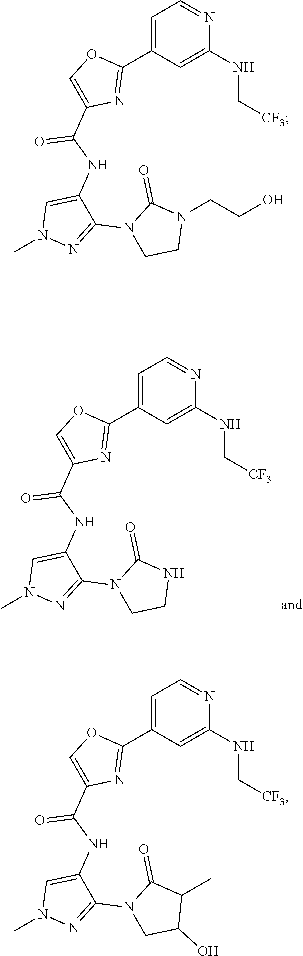 [SMILES: Cn1cc(NC(=O)c2coc(-c3ccnc(NCC(F)(F)F)c3)n2)c(N2CCN(CCO)C2=O)n1 ; Cn1cc(NC(=O)c2coc(-c3ccnc(NCC(F)(F)F)c3)n2)c(N2CCNC2=O)n1 ; CC1C(=O)N(c2nn(C)cc2NC(=O)c2coc(-c3ccnc(NCC(F)(F)F)c3)n2)CC1O]

or a stereoisomer, solvates, tautomers, or pharmaceutically acceptable salts thereof.
2.43 Compound 2.42, wherein the compound of Formula III has the following structure:

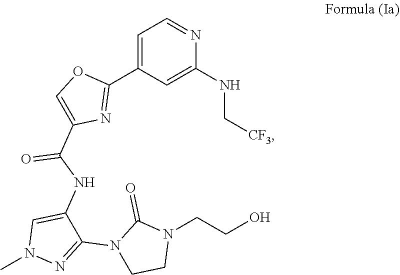 [SMILES: Cn1cc(NC(=O)c2coc(-c3ccnc(NCC(F)(F)F)c3)n2)c(N2CCN(CCO)C2=O)n1]

or is a stereoisomer, solvate, tautomer, or a pharmaceutically acceptable salt thereof.
2.44 Compound 2.42, wherein the compound of Formula III has the following structure:

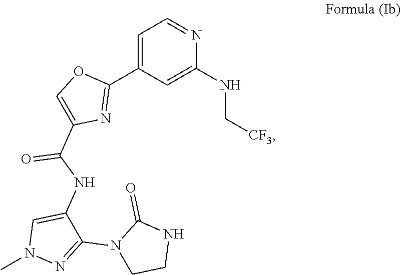 [SMILES: Cn1cc(NC(=O)c2coc(-c3ccnc(NCC(F)(F)F)c3)n2)c(N2CCNC2=O)n1]

or is a stereoisomer, solvate, tautomer, or a pharmaceutically acceptable salt thereof.
2.45 Compound 2.42, wherein the compound of Formula III has the following structure:

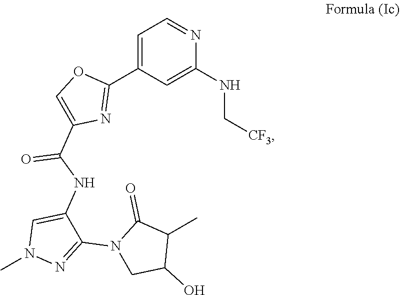 [SMILES: CC1C(=O)N(c2nn(C)cc2NC(=O)c2coc(-c3ccnc(NCC(F)(F)F)c3)n2)CC1O]

or is a stereoisomer, solvate, tautomer, or a pharmaceutically acceptable salt thereof.

In several embodiments, the present disclosure provides for a topical composition [Composition 1] comprising a compound of Formula I, II (e.g., Compound 1, et seq.), or III (e.g., Compound 2, et seq.) and a pharmaceutically acceptable vehicle.

The present disclosure further provides compositions as follows:
1.1 Composition 1, wherein the composition comprises a compound of Formula III (e.g. Compound 2, et seq.).
1.2 Composition 1 or 1.1, wherein the composition is in the form of a cream, a gel, a spray or an ointment.
1.3 Any of the preceding compositions, wherein the compound of Formula (I) is at a concentration of about 0.001 wt. % to about 10 wt. %.
1.4 Any of the preceding compositions, wherein the compound of Formula (I) is at a concentration of about 0.1 wt. % to about 5 wt. %.
1.5 Any of the preceding compositions, further comprising a skin penetration enhancer, adjuvant, carrier, vehicle (e.g. liposome), solvent, co-solvent, preservatives, viscosity enhancers, pH adjusters, film-forming agents, humectant, glidant, sweetening agent, diluent, preservative, dye/colorant, flavor enhancer, surfactant, wetting agent, dispersing agent, suspending agent, stabilizer, isotonic agent, solvent, emulsifier, anti-oxidant, or chelating agent.
1.6 The preceding composition, wherein the skin penetration enhancer comprises one or more of mannitol, sulphoxides (e.g., dimethylsulphoxide, DMSO), Azones (e.g. laurocapram), pyrrolidones (e.g., 2-pyrrolidone, 2P), alcohols and alkanols (e.g., ethanol, or decanol), glycols (e.g., propylene glycol, hexylene glycol, polyoxyethylene glycol, diethylene glycol), surfactants (also common in dosage forms) and terpenes.
1.7 The preceding composition, wherein the composition is applied to a patient's skin once daily, every other day, weekly or monthly.
1.8 The preceding composition, wherein the composition is applied to a patient's skin twice daily.
1.9 The preceding composition, wherein the composition is applied to a patient's skin three times daily or more.
1.10 Any of the preceding compositions, wherein the composition is administered to a patient suffering from a dermatological disorder.
1.11 Composition 1.10, wherein the dermatological disorder is an inflammatory dermatological disorder.
1.12 The preceding composition, wherein the inflammatory dermatological disorder is rosacea, psoriasis, atopic dermatitis, hidradenitis suppurativa, seborrheic dermatitis, contact dermatitis, urticaria, dermatitis herpetiformis, nummular dermatitis, lichen planus, pityriasis rosea, cutaneous lupus, acne, cancers of the skin (e.g. cutaneous T-cell lymphoma), or miliaria.
1.13 Any of compositions 1.10-1.12, wherein the inflammatory dermatological disorder is rosacea.
1.14 The preceding composition, wherein the rosacea is papulopustular rosacea.
1.15 Any of compositions 1.10-1.12, wherein the inflammatory dermatological disorder is psoriasis.
1.16 Any of compositions 1.10-1.12, wherein the inflammatory dermatological disorder is atopic dermatitis.
1.17 Any of compositions 1.10-1.12, wherein inflammatory dermatological disorder is hidradenitis suppurativa.
1.18 Any of compositions 1.10-1.12, wherein inflammatory dermatological disorder is cutaneous lupus.
1.19 Any of compositions 1.10-1.12, wherein inflammatory dermatological disorder is acne.
1.20 Any of compositions 1.10-1.12, wherein inflammatory dermatological disorder is a cancer of the skin (e.g., cutaneous T-cell lymphoma).
1.21
1.22 Any of the preceding compositions, the subject is a human.
1.23 Any of the preceding compositions, wherein the mammalian skin is human skin.

The IRAK4 inhibitors described herein may be prepared according to the methods disclosed in, for example, U.S. Pat. Nos. 9,890,145 and 9,321,757, which patents are incorporated by reference in their entireties.

The definition of each substituent used in the present specification is described in detail in the following. Unless otherwise specified, each substituent has the following definition.

In the present specification, examples of the “halogen atom” include fluorine, chlorine, bromine and iodine.

In the present specification, examples of the “C₁₋₆ alkyl group” include methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl and 2-ethylbutyl.

In the present specification, examples of the “optionally halogenated C₁₋₆ alkyl group” include a C1-6 alkyl group optionally having 1 to 7, preferably 1 to 5 halogen atoms. Specific examples thereof include methyl, chloromethyl, difluoromethyl, trichloromethyl, trifluoromethyl, ethyl, 2-bromoethyl, 2,2,2-trifluoroethyl, tetrafluoroethyl, pentafluoroethyl, propyl, 2,2-difluoropropyl, 3,3,3-trifluoropropyl, isopropyl, butyl, 4,4,4-trifluorobutyl, isobutyl, sec-butyl, tert-butyl, pentyl, isopentyl, neopentyl, 5,5,5-trifluoropentyl, hexyl and 6,6,6-trifluorohexyl.

In the present specification, examples of the “C₂₋₆ alkenyl group” include ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl and 5-hexenyl.

In the present specification, examples of the “C₂₋₆ alkynyl group” include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and 4-methyl-2-pentynyl.

In the present specification, examples of the “C₃₋₁₀cycloalkyl group” include cyclopropyl, cyclobutyl, cyclopentyl, cyclohexyl, cycloheptyl, cyclooctyl, bicyclo[2.2.1]heptyl, bicyclo[2.2.2]octyl, bicyclo[3.2.1]octyl and adamantyl.

In the present specification, examples of the “optionally halogenated C₃₋₁₀ cycloalkyl group” include a C₃₋₁₀ cycloalkyl group optionally having 1 to 7, preferably 1 to 5 halogen atoms. Specific examples thereof include cyclopropyl, 2,2-difluorocyclopropyl, 2,3-difluorocyclopropyl, cyclobutyl, difluorocyclobutyl, cyclopentyl, cyclohexyl, cycloheptyl and cyclooctyl.

In the present specification, examples of the “C₃₋₁₀ cycloalkenyl group” include cyclopropenyl, cyclobutenyl, cyclopentenyl, cyclohexenyl, cycloheptenyl and cyclooctenyl.

In the present specification, examples of the “C₆₋₁₄ aryl group” include phenyl, 1-naphthyl, 2-naphthyl, 1-anthryl, 2-anthryl and 9-anthryl.

In the present specification, examples of the “C₇₋₁₆ aralkyl group” include benzyl, phenethyl, naphthylmethyl and phenylpropyl.

In the present specification, examples of the “C₁₋₆ alkoxy group” include methoxy, ethoxy, propoxy, isopropoxy, butoxy, isobutoxy, sec-butoxy, tert-butoxy, pentyloxy and hexyloxy.

In the present specification, examples of the “optionally halogenated C₁₋₆ alkoxy, group” include a C₁₋₆ alkoxy group optionally having 1 to 7, preferably 1 to 5 halogen atoms. Specific examples thereof include methoxy, difluoromethoxy, trifluoromethoxy, ethoxy, 2,2,2-trifluoroethoxy, propoxy, isopropoxy, butoxy, 4,4,4-trifluorobutoxy, isobutoxy, sec-butoxy, pentyloxy and hexyloxy.

In the present specification, examples of the “C₃₋₁₀ cycloalkyloxy group” include cyclopropyloxy, cyclobutyloxy, cyclopentyloxy, cyclohexyloxy, cycloheptyloxy and cyclooctyloxy.

In the present specification, examples of the “C₁₋₆ alkylthio group” include methylthio, ethylthio, propylthio, isopropylthio, butylthio, sec-butylthio, tert-butylthio, pentylthio and hexylthio.

In the present specification, examples of the “optionally halogenated C₁₋₆ alkylthio group” include a C₁₋₆ alkylthio group optionally having 1 to 7, preferably 1 to 5 halogen atoms. Specific examples thereof include methylthio, difluoromethylthio, trifluoromethylthio, ethylthio, propylthio, isopropylthio, butylthio, 4,4,4-trifluorobutylthio, pentylthio and hexylthio.

In the present specification, examples of the “C₁₋₆ alkyl-carbonyl group” include acetyl, propanoyl, butanoyl, 2-methylpropanoyl, pentanoyl, 3-methylbutanoyl, 2-methylbutanoyl, 2,2-dimethylpropanoyl, hexanoyl and heptanoyl.

In the present specification, examples of the “optionally halogenated C₁₋₆ alkyl-carbonyl group” include a C₁₋₆ alkyl-carbonyl group optionally having 1 to 7, preferably 1 to 5 halogen atoms. Specific examples thereof include acetyl, chloroacetyl, trifluoroacetyl, trichloroacetyl, propanoyl, butanoyl, pentanoyl and hexanoyl.

In the present specification, examples of the “C₁₋₆ alkoxy-carbonyl group” include methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, pentyloxycarbonyl and hexyloxycarbonyl.

In the present specification, examples of the “C₆₋₁₄ aryl-carbonyl group” include benzoyl, 1-naphthoyl and 2-naphthoyl.

In the present specification, examples of the “C₇₋₁₆ aralkyl-carbonyl group” include phenylacetyl and phenylpropionyl.

In the present specification, examples of the “5- to 14-membered aromatic heterocyclylcarbonyl group” include nicotinoyl, isonicotinoyl, thenoyl and furoyl.

In the present specification, examples of the “3- to 14-membered non-aromatic heterocyclylcarbonyl group” include morpholinylcarbonyl, piperidinylcarbonyl and pyrrolidinylcarbonyl.

In the present specification, examples of the “mono- or di-C₁₋₆ alkyl-carbamoyl group” include methylcarbamoyl, ethylcarbamoyl, dimethylcarbamoyl, diethylcarbamoyl and N-ethyl-N-methylcarbamoyl.

In the present specification, examples of the “mono- or di-C₇₋₁₆ aralkyl-carbamoyl group” include benzylcarbamoyl and phenethylcarbamoyl.

In the present specification, examples of the “C₁₋₆ alkylsulfonyl group” include methylsulfonyl, ethylsulfonyl, propylsulfonyl, isopropylsulfonyl, butylsulfonyl, sec-butylsulfonyl and tert-butylsulfonyl.

In the present specification, examples of the “optionally halogenated C₁₋₆ alkylsulfonyl group” include a C₁₋₆ alkylsulfonyl group optionally having 1 to 7, preferably 1 to 5 halogen atoms. Specific examples thereof include methylsulfonyl, difluoromethylsulfonyl, trifluoromethylsulfonyl, ethylsulfonyl, propylsulfonyl, isopropylsulfonyl, butylsulfonyl, 4,4,4-trifluorobutylsulfonyl, pentylsulfonyl and hexylsulfonyl.

In the present specification, examples of the “C₆₋₁₄ arylsulfonyl group” include phenylsulfonyl, 1-naphthylsulfonyl and 2-naphthylsulfonyl.

In the present specification, examples of the “substituent” include a halogen atom, a cyano group, a nitro group, an optionally substituted hydrocarbon group, an optionally substituted heterocyclic group, an acyl group, an optionally substituted amino group, an optionally substituted carbamoyl group, an optionally substituted thiocarbamoyl group, an optionally substituted sulfamoyl group, an optionally substituted hydroxy group, an optionally substituted sulfanyl (SH) group and an optionally substituted silyl group.

In the present specification, examples of the “hydrocarbon group” (including “hydrocarbon group” of “optionally substituted hydrocarbon group”) include a C₁₋₆ alkyl group, a C₂₋₆ alkenyl group, a C₂₋₆ alkynyl group, a C₃₋₁₀ cycloalkyl group, a C₃₋₄₀ cycloalkenyl group, a C₆₋₁₄ aryl group and a C₇₋₁₆ aralkyl group.

In the present specification, examples of the “optionally substituted hydrocarbon group” include a hydrocarbon group optionally having substituent(s) selected from the following Substituent Group A.

[Substituent Group A]
(1) a halogen atom,
(2) a nitro group,
(3) a cyano group,
(4) an oxo group,
(5) a hydroxy group,
(6) an optionally halogenated C₁₋₆ alkoxy group,
(7) a C₆₋₁₄ aryloxy group (e.g., phenoxy, naphthoxy),
(8) a C₇₋₁₆ aralkyloxy group (e.g., benzyloxy),
(9) a 5- to 14-membered aromatic heterocyclyloxy group (e.g., pyridyloxy),
(10) a 3- to 14-membered non-aromatic heterocyclyloxy group (e.g., morpholinyloxy, piperidinyloxy),
(11) a C₁₋₆ alkyl-carbonyloxy group (e.g., acetoxy, propanoyloxy),
(12) a C₆₋₁₄ aryl-carbonyloxy group (e.g., benzoyloxy, naphthoyloxy, 2-naphthoyloxy),
(13) a C₁₋₆ alkoxy-carbonyloxy group (e.g., methoxycarbonyloxy, ethoxycarbonyloxy, propoxycarbonyloxy, butoxycarbonyloxy),
(14) a mono- or di-C₁₋₆alkyl-carbamoyloxy group (e.g., methylcarbamoyloxy, ethylcarbamoyloxy, dimethylcarbamoyloxy, diethylcarbamoyloxy),
(15) a C₆₋₁₄ aryl-carbamoyloxy group (e.g., phenylcarbamoyloxy, naphthylcarbamoyloxy),
(16) a 5- to 14-membered aromatic heterocyclylcarbonyloxy group (e.g., nicotinoyloxy),
(17) a 3- to 14-membered non-aromatic heterocyclylcarbonyloxy group (e.g., morpholinylcarbonyloxy, piperidinylcarbonyloxy),
(18) an optionally halogenated C₁₋₆ alkylsulfonyloxy group (e.g., methylsulfonyloxy, trifluoromethylsulfonyloxy),
(19) a C₆₋₁₄ arylsulfonyloxy group optionally substituted by a C₁₋₆ alkyl group (e.g., phenylsulfonyloxy, toluenesulfonyloxy),
(20) an optionally halogenated C₁₋₆ alkylthio group,
(21) a 5- to 14-membered aromatic heterocyclic group,
(22) a 3- to 14-membered non-aromatic heterocyclic group,
(23) a formyl group,
(24) a carboxy group,
(25) an optionally halogenated C₁₋₆ alkyl-carbonyl group,
(26) a C₆₋₁₄ aryl-carbonyl group,
(27) a 5- to 14-membered aromatic heterocyclylcarbonyl group,
(28) a 3- to 14-membered non-aromatic heterocyclylcarbonyl group,
(29) a C₁₋₆ alkoxy-carbonyl group,
(30) a C₆₋₁₄ aryloxy-carbonyl group (e.g., phenyloxycarbonyl, 1-naphthyloxycarbonyl, 2-naphthyloxycarbonyl),
(31) a C₇₋₁₆ aralkyloxy-carbonyl group (e.g., benzyloxycarbonyl, phenethyloxycarbonyl),
(32) a carbamoyl group,
(33) a thiocarbamoyl group,
(34) a mono- or di-C₁₋₆ alkyl-carbamoyl group,
(35) a C₆₋₁₄ aryl-carbamoyl group (e.g., phenylcarbamoyl),
(36) a 5- to 14-membered aromatic heterocyclylcarbamoyl group (e.g., pyridylcarbamoyl, thienylcarbamoyl),
(37) a 3- to 14-membered non-aromatic heterocyclylcarbamoyl group (e.g., morpholinylcarbamoyl, piperidinylcarbamoyl),
(38) an optionally halogenated C₁₋₆ alkylsulfonyl group,
(39) a C₆₋₁₄ arylsulfonyl group,
(40) a 5- to 14-membered aromatic heterocyclylsulfonyl group (e.g., pyridylsulfonyl, thienylsulfonyl),
(41) an optionally halogenated C₁₋₆ alkylsulfinyl group,
(42) a C₆₋₁₄ arylsulfinyl group (e.g., phenylsulfinyl, 1-naphthylsulfinyl, 2-naphthylsulfinyl),
(43) a 5- to 14-membered aromatic heterocyclylsulfinyl group (e.g., pyridylsulfinyl, thienylsulfinyl),
(44) an amino group,
(45) a mono- or di-C₁₋₆ alkylamino group (e.g., methylamino, ethylamino, propylamino, isopropylamino, butylamino, dimethylamino, diethylamino, dipropylamino, dibutylamino, N-ethyl-N-methylamino),
(46) a mono- or di-C₆₋₁₄ acylamino group (e.g., phenylamino),
(47) a 5- to 14-membered aromatic heterocyclylamino group (e.g., pyridylamino),
(48) a C₇₋₁₆ aralkylamino group (e.g., benzylamino),
(49) a formylamino group,
(50) a C₁₋₆ alkyl-carbonylamino group (e.g., acetylamino, propanoylamino, butanoylamino),
(51) a (C₁₋₆ alkyl) (C₁₋₆ alkyl-carbonyl)amino group (e.g., N-acetyl-N-methylamino),
(52) a C₆₋₁₄ aryl-carbonylamino group (e.g., phenylcarbonylamino, naphthylcarbonylamino),
(53) a C₁₋₆ alkoxy-carbonylamino group (e.g., methoxycarbonylamino, ethoxycarbonylamino, propoxycarbonylamino, butoxycarbonylamino, tert-butoxycarbonylamino),
(54) a C₇₋₁₆ aralkyloxy-carbonylamino group (e.g., benzyloxycarbonylamino),
(55) a C₁₋₆ alkylsulfonylamino group (e.g., methylsulfonylamino, ethylsulfonylamino),
(56) a C₆₋₁₄ arylsulfonylamino group optionally substituted by a C₁₋₆ alkyl group (e.g., phenylsulfonylamino, toluenesulfonylamino),
(57) an optionally halogenated C₁₋₆ alkyl group,
(58) a C₂₋₆ alkenyl group,
(59) a C₂₋₆ alkynyl group,
(60) a C₃₋₁₀ cycloalkyl group,
(61) a C₃₋₁₀ cycloalkenyl group and
(62) a C₆₋₁₄ aryl group.

The number of the above-mentioned substituents in the “optionally substituted hydrocarbon group” is, for example, 1 to 5, preferably 1 to 3. When the number of the substituents is two or more, the respective substituents may be the same or different.

In the present specification, examples of the “heterocyclic group” (including “heterocyclic group” of “optionally substituted heterocyclic group”) include (i) an aromatic heterocyclic group, (ii) a non-aromatic heterocyclic group and (iii) a 7- to 10-membered bridged heterocyclic group, each containing, as a ring-constituting atom besides carbon atom, 1 to 4 hetero atoms selected from a nitrogen atom, a sulfur atom and an oxygen atom.

In the present specification, examples of the “aromatic heterocyclic group” (including “5- to 14-membered aromatic heterocyclic group”) include a 5- to 14-membered (preferably 5- to 10-membered) aromatic heterocyclic group containing, as a ring-constituting atom besides carbon atom, 1 to 4 hetero atoms selected from a nitrogen atom, a sulfur atom and an oxygen atom.

Preferable examples of the “aromatic heterocyclic group” include 5- or 6-membered monocyclic aromatic heterocyclic groups such as thienyl, furyl, pyrrolyl, imidazolyl, pyrazolyl, thiazolyl, isothiazolyl, oxazolyl, isoxazolyl, pyridyl, pyrazinyl, pyrimidinyl, pyridazinyl, 1,2,4-oxadiazolyl, 1,3,4-oxadiazolyl, 1,2,4-thiadiazolyl, 1,3,4-thiadiazolyl, triazolyl, tetrazolyl, triazinyl and the like; and

8- to 14-membered fused polycyclic (preferably bi or tricyclic) aromatic heterocyclic groups such as benzothiophenyl, benzofuranyl, benzimidazolyl, benzoxazolyl, benzisoxazolyl, benzothiazolyl, benzisothiazolyl, benzotriazolyl, imidazopyridinyl, thienopyridinyl, furopyridinyl, pyrrolopyridinyl, pyrazolopyridinyl, oxazolopyridinyl, thiazolopyridinyl, imidazopyrazinyl, imidazopyrimidinyl, thienopyrimidinyl, furopyrimidinyl, pyrrolopyrimidinyl, pyrazolopyrimidinyl, oxazolopyrimidinyl, thiazolopyrimidinyl, pyrazolotriazinyl, naphtho[2,3-b]thienyl, phenoxathiinyl, indolyl, isoindolyl, 1H-indazolyl, purinyl, isoquinolyl, quinolyl, phthalazinyl, naphthyridinyl, quinoxalinyl, quinazolinyl, cinnolinyl, carbazolyl, O-carbolinyl, phenanthridinyl, acridinyl, phenazinyl, phenothiazinyl, phenoxazinyl and the like.

In the present specification, examples of the “non-aromatic heterocyclic group” (including “3- to 14-membered non-aromatic heterocyclic group”) include a 3- to 14-membered (preferably 4- to 10-membered) non-aromatic heterocyclic group containing, as a ring-constituting atom besides carbon atom, 1 to 4 hetero atoms selected from a nitrogen atom, a sulfur atom and an oxygen atom.

Preferable examples of the “non-aromatic heterocyclic group” include 3- to 8-membered monocyclic non-aromatic heterocyclic groups such as aziridinyl, oxiranyl, thiiranyl, azetidinyl, oxetanyl, thietanyl, tetrahydrothienyl, tetrahydrofuranyl, pyrrolinyl, pyrrolidinyl, imidazolinyl, imidazolidinyl, oxazolinyl, oxazolidinyl, pyrazolinyl, pyrazolidinyl, thiazolinyl, thiazolidinyl, tetrahydroisothiazolyl, tetrahydrooxazolyl, tetrahydroisooxazolyl, piperidinyl, piperazinyl, tetrahydropyridinyl, dihydropyridinyl, dihydrothiopyranyl, tetrahydropyrimidinyl, tetrahydropyridazinyl, dihydropyranyl, tetrahydropyranyl, tetrahydrothiopyranyl, morpholinyl, thiomorpholinyl, azepanyl, diazepanyl, azepinyl, oxepanyl, azocanyl, diazocanyl and the like; and

9- to 14-membered fused polycyclic (preferably bi or tricyclic) non-aromatic heterocyclic groups such as dihydrobenzofuranyl, dihydrobenzimidazolyl, dihydrobenzoxazolyl, dihydrobenzothiazolyl, dihydrobenzisothiazolyl, dihydronaphtho[2,3-b]thienyl, tetrahydroisoquinolyl, tetrahydroquinolyl, 4H-quinolizinyl, indolinyl, isoindolinyl, tetrahydrothieno[2,3-c]pyridinyl, tetrahydrobenzazepinyl, tetrahydroquinoxalinyl, tetrahydrophenanthridinyl, hexahydrophenothiazinyl, hexahydrophenoxazinyl, tetrahydrophthalazinyl, tetrahydronaphthyridinyl, tetrahydroquinazolinyl, tetrahydrocinnolinyl, tetrahydrocarbazolyl, tetrahydro-β-carbolinyl, tetrahydroacrydinyl, tetrahydrophenazinyl, tetrahydrothioxanthenyl, octahydroisoquinolyl and the like.

In the present specification, preferable examples of the “7- to 10-membered bridged heterocyclic group” include quinuclidinyl and 7-azabicyclo[2.2.1]heptanyl.

In the present specification, examples of the “nitrogen-containing heterocyclic group” include a “heterocyclic group” containing at least one nitrogen atom as a ring-constituting atom.

In the present specification, examples of the “optionally substituted heterocyclic group” include a heterocyclic group optionally having substituent(s) selected from the above-mentioned Substituent Group A.

The number of the substituents in the “optionally substituted heterocyclic group” is, for example, 1 to 3. When the number of the substituents is two or more, the respective substituents may be the same or different.

In the present specification, examples of the “acyl group” include a formyl group, a carboxy group, a carbamoyl group, a thiocarbamoyl group, a sulfino group, a sulfo group, a sulfamoyl group and a phosphono group, each optionally having “1 or 2 substituents selected from a C₁₋₆ alkyl group, a C₂₋₆ alkenyl group, a C₃₋₁₀cycloalkyl group, a C₃₋₁₀cycloalkenyl group, a C₆₋₁₄ aryl group, a C₇₋₁₆ aralkyl group, a 5- to 14-membered aromatic heterocyclic group and a 3- to 14-membered non-aromatic heterocyclic group, each of which optionally has 1 to 3 substituents selected from a halogen atom, an optionally halogenated C₁₋₆ alkoxy group, a hydroxy group, a nitro group, a cyano group, an amino group and a carbamoyl group”.

Examples of the “acyl group” also include a hydrocarbon-sulfonyl group, a heterocyclylsulfonyl group, a hydrocarbon-sulfinyl group and a heterocyclylsulfinyl group.

Here, the hydrocarbon-sulfonyl group means a hydrocarbon group-bonded sulfonyl group, the heterocyclylsulfonyl group means a heterocyclic group-bonded sulfonyl group, the hydrocarbon-sulfinyl group means a hydrocarbon group-bonded sulfinyl group and the heterocyclylsulfinyl group means a heterocyclic group-bonded sulfinyl group.

Preferable examples of the “acyl group” include a formyl group, a carboxy group, a C₁₋₆ alkyl-carbonyl group, a C₂₋₆ alkenyl-carbonyl group (e.g., crotonoyl), a C₃₋₁₀ cycloalkyl-carbonyl group (e.g., cyclobutanecarbonyl, cyclopentanecarbonyl, cyclohexanecarbonyl, cycloheptanecarbonyl), a C₃₋₁₀ cycloalkenyl-carbonyl group (e.g., 2-cyclohexenecarbonyl), a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a C₆₋₁₄ aryloxy-carbonyl group (e.g., phenyloxycarbonyl, naphthyloxycarbonyl), a C₇₋₁₆ aralkyloxy-carbonyl group (e.g., benzyloxycarbonyl, phenethyloxycarbonyl), a carbamoyl group, a mono- or di-C₁₋₆ alkyl-carbamoyl group, a mono- or di-C₂₋₆ alkenyl-carbamoyl group (e.g., diallylcarbamoyl), a mono- or di-C₃₋₁₀ cycloalkyl-carbamoyl group (e.g., cyclopropylcarbamoyl), a mono- or di-C₆₋₁₄ aryl-carbamoyl group (e.g., phenylcarbamoyl), a mono- or di-C₇₋₁₆ aralkyl-carbamoyl group, a 5- to 14-membered aromatic heterocyclylcarbamoyl group (e.g., pyridylcarbamoyl), a thiocarbamoyl group, a mono- or di-C₁₋₆ alkyl-thiocarbamoyl group (e.g., methylthiocarbamoyl, N-ethyl-N-methylthiocarbamoyl), a mono- or di-C₂₋₆ alkenyl-thiocarbamoyl group (e.g., diallylthiocarbamoyl), a mono- or di-C₃₋₁₀ cycloalkyl-thiocarbamoyl group (e.g., cyclopropylthiocarbamoyl, cyclohexylthiocarbamoyl), a mono- or di-C₆₋₁₄ aryl-thiocarbamoyl group (e.g., phenylthiocarbamoyl), a mono- or di-C₇₋₁₆ aralkyl-thiocarbamoyl group (e.g., benzylthiocarbamoyl, phenethylthiocarbamoyl), a 5- to 14-membered aromatic heterocyclylthiocarbamoyl group (e.g., pyridylthiocarbamoyl), a sulfino group, a C₁₋₆ alkylsulfinyl group (e.g., methylsulfinyl, ethylsulfinyl), a sulfo group, a C₁₋₆ alkylsulfonyl group, a C₆₋₁₄ arylsulfonyl group, a phosphono group and a mono- or di-C₁₋₆ alkylphosphono group (e.g., dimethylphosphono, diethylphosphono, diisopropylphosphono, dibutylphosphono).

In the present specification, examples of the “optionally substituted amino group” include an amino group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group, a mono- or di-C₇₋₁₆ aralkyl-carbamoyl group, a C₁₋₆ alkylsulfonyl group and a C₆₋₁₄ arylsulfonyl group, each of which optionally has 1 to 3 substituents selected from Substituent Group A”.

Preferable examples of the optionally substituted amino group include an amino group, a mono- or di-(optionally halogenated C₁₋₆ alkyl)amino group (e.g., methylamino, trifluoromethylamino, dimethylamino, ethylamino, diethylamino, propylamino, dibutylamino), a mono- or di-C₂₋₆ alkenylamino group (e.g., diallylamino), a mono- or di-C₃₋₁₀ cycloalkylamino group (e.g., cyclopropylamino, cyclohexylamino), a mono- or di-C₆₋₁₄ arylamino group (e.g., phenylamino), a mono- or di-C₇₋₁₆ aralkylamino group (e.g., benzylamino, dibenzylamino), a mono- or di-(optionally halogenated C₁₋₆ alkyl)-carbonylamino group (e.g., acetylamino, propionylamino), a mono- or di-C₆₋₁₄ aryl-carbonylamino group (e.g., benzoylamino), a mono- or di-C₇₋₁₆ aralkyl-carbonylamino group (e.g., benzylcarbonylamino), a mono- or di-5- to 14-membered aromatic heterocyclylcarbonylamino group (e.g., nicotinoylamino, isonicotinoylamino), a mono- or di-3- to 14-membered non-aromatic heterocyclylcarbonylamino group (e.g., piperidinylcarbonylamino), a mono- or di-C₁₋₆ alkoxy-carbonylamino group (e.g., tert-butoxycarbonylamino), a 5- to 14-membered aromatic heterocyclylamino group (e.g., pyridylamino), a carbamoylamino group, a (mono- or di-C₁₋₆ alkyl-carbamoyl)amino group (e.g., methylcarbamoylamino), a (mono- or di-C₇₋₁₆ aralkyl-carbamoyl)amino group (e.g., benzylcarbamoylamino), a C₁₋₆ alkylsulfonylamino group (e.g., methylsulfonylamino, ethylsulfonylamino), a C₆₋₁₄ arylsulfonylamino group (e.g., phenylsulfonylamino), a (C₁₋₆ alkyl) (C₁₋₆ alkyl-carbonyl)amino group (e.g., N-acetyl-N-methylamino) and a (C₁₋₆ alkyl) (C₆₋₁₄ aryl-carbonyl)amino group (e.g., N-benzoyl-N-methylamino).

In the present specification, examples of the “optionally substituted carbamoyl group” include a carbamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group and a mono- or di-C₇₋₁₆ aralkyl-carbamoyl group, each of which optionally has 1 to 3 substituents selected from Substituent Group A”.

Preferable examples of the optionally substituted carbamoyl group include a carbamoyl group, a mono- or di-C₁₋₆ alkyl-carbamoyl group, a mono- or di-C₂₋₆ alkenyl-carbamoyl group (e.g., diallylcarbamoyl), a mono- or di-C₃₋₁₀cycloalkyl-carbamoyl group (e.g., cyclopropylcarbamoyl, cyclohexylcarbamoyl), a mono- or di-C₆₋₁₄ aryl-carbamoyl group (e.g., phenylcarbamoyl), a mono- or di-C₇₋₁₆ aralkyl-carbamoyl group, a mono- or di-C₁₋₆ alkyl-carbonyl-carbamoyl group (e.g., acetylcarbamoyl, propionylcarbamoyl), a mono- or di-C₆₋₁₄ carbonyl-carbamoyl group (e.g., benzoylcarbamoyl) and a 5- to 14-membered aromatic heterocyclylcarbamoyl group (e.g., pyridylcarbamoyl).

In the present specification, examples of the “optionally substituted thiocarbamoyl group” include a thiocarbamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group and a mono- or di-C₇₋₁₆ aralkyl-carbamoyl group, each of which optionally has 1 to 3 substituents selected from Substituent Group A”.

Preferable examples of the optionally substituted thiocarbamoyl group include a thiocarbamoyl group, a mono- or alkyl-thiocarbamoyl group (e.g., methylthiocarbamoyl, ethylthiocarbamoyl, dimethylthiocarbamoyl, diethylthiocarbamoyl, N-ethyl-N-methylthiocarbamoyl), a mono- or di-C₂₋₆ alkenyl-thiocarbamoyl group (e.g., diallylthiocarbamoyl), a mono- or di-C₃₋₁₀ cycloalkyl-thiocarbamoyl group (e.g., cyclopropylthiocarbamoyl, cyclohexylthiocarbamoyl), a mono- or di-C₆₋₁₄ aryl-thiocarbamoyl group (e.g., phenylthiocarbamoyl), a mono- or di-C₇₋₁₆ aralkyl-thiocarbamoyl group (e.g., benzylthiocarbamoyl, phenethylthiocarbamoyl), a mono- or di-C₁₋₆ alkyl-carbonyl-thiocarbamoyl group (e.g., acetylthiocarbamoyl, propionylthiocarbamoyl), a mono- or di-C₆₋₁₄ aryl-carbonyl-thiocarbamoyl group (e.g., benzoylthiocarbamoyl) and a 5- to 14-membered aromatic heterocyclylthiocarbamoyl group (e.g., pyridylthiocarbamoyl).

In the present specification, examples of the “optionally substituted sulfamoyl group” include a sulfamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group and a mono- or di-C746 aralkyl-carbamoyl group, each of which optionally has 1 to 3 substituents selected from Substituent Group A”.

Preferable examples of the optionally substituted sulfamoyl group include a sulfamoyl group, a mono- or di-C₁₋₆ alkyl-sulfamoyl group (e.g., methylsulfamoyl, ethylsulfamoyl, dimethylsulfamoyl, diethylsulfamoyl, N-ethyl-N-methylsulfamoyl), a mono- or di-C₂₋₆ alkenyl-sulfamoyl group (e.g., diallylsulfamoyl), a mono- or di-C₃₋₁₀ cycloalkyl-sulfamoyl group (e.g., cyclopropylsulfamoyl, cyclohexylsulfamoyl), a mono- or di-C₆₋₁₄ aryl-sulfamoyl group (e.g., phenylsulfamoyl), a mono- or di-C₇₋₁₆ aralkyl-sulfamoyl group (e.g., benzylsulfamoyl, phenethylsulfamoyl), a mono- or alkyl-carbonyl-sulfamoyl group (e.g., acetylsulfamoyl, propionylsulfamoyl), a mono- or di-C₆₋₁₄ aryl-carbonyl-sulfamoyl group (e.g., benzoylsulfamoyl) and a 5- to 14-membered aromatic heterocyclylsulfamoyl group (e.g., pyridylsulfamoyl).

In the present specification, examples of the “optionally substituted hydroxy group” include a hydroxyl group optionally having “a substituent selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group, a mono- or di-C₇₋₁₆ aralkyl-carbamoyl group, a C₁₋₆ alkylsulfonyl group and a C₆₋₁₄ arylsulfonyl group, each of which optionally has 1 to 3 substituents selected from Substituent Group A”.

Preferable examples of the optionally substituted hydroxy group include a hydroxy group, a C₁₋₆ alkoxy group, a C₂₋₆ alkenyloxy group (e.g., allyloxy, 2-butenyloxy, 2-pentenyloxy, 3-hexenyloxy), a C₃₋₁₀ cycloalkyloxy group (e.g., cyclohexyloxy), a C₆₋₁₄ aryloxy group (e.g., phenoxy, naphthyloxy), a C₇₋₁₆ aralkyloxy group (e.g., benzyloxy, phenethyloxy), a C₁₋₆ alkyl-carbonyloxy group (e.g., acetyloxy, propionyloxy, butyryloxy, isobutyryloxy, pivaloyloxy), a C₆₋₁₄ aryl-carbonyloxy group (e.g., benzoyloxy), a C₇₋₁₆ aralkyl-carbonyloxy group (e.g., benzylcarbonyloxy), a 5- to 14-membered aromatic heterocyclylcarbonyloxy group (e.g., nicotinoyloxy), a 3- to 14-membered non-aromatic heterocyclylcarbonyloxy group (e.g., piperidinylcarbonyloxy), a C₁₋₆ alkoxy-carbonyloxy group (e.g., tert-butoxycarbonyloxy), a 5- to 14-membered aromatic heterocyclyloxy group (e.g., pyridyloxy), a carbamoyloxy group, a C₁₋₆ alkyl-carbamoyloxy group (e.g., methylcarbamoyloxy), a C₇₋₁₆ aralkyl-carbamoyloxy group (e.g., benzylcarbamoyloxy), a C₁₋₆ alkylsulfonyloxy group (e.g., methylsulfonyloxy, ethylsulfonyloxy) and a C₆₋₁₄ arylsulfonyloxy group (e.g., phenylsulfonyloxy).

In the present specification, examples of the “optionally substituted sulfanyl group” include a sulfanyl group optionally having “a substituent selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group and a 5- to 14-membered aromatic heterocyclic group, each of which optionally has 1 to 3 substituents selected from Substituent Group A” and a halogenated sulfanyl group.

Preferable examples of the optionally substituted sulfanyl group include a sulfanyl (—SH) group, a C₁₋₆ alkylthio group, a C₂₋₆ alkenylthio group (e.g., allylthio, 2-butenylthio, 2-pentenylthio, 3-hexenylthio), a C₃₋₁₀ cycloalkylthio group (e.g., cyclohexylthio), a C₆₋₁₄ arylthio group (e.g., phenylthio, naphthylthio), a C₇₋₁₆ aralkylthio group (e.g., benzylthio, phenethylthio), a C₁₋₆ alkyl-carbonylthio group (e.g., acetylthio, propionylthio, butyrylthio, isobutyrylthio, pivaloylthio), a C₆₋₁₄ aryl-carbonylthio group (e.g., benzoylthio), a 5- to 14-membered aromatic heterocyclylthio group (e.g., pyridylthio) and a halogenated thio group (e.g., pentafluorothio).

In the present specification, examples of the “optionally substituted silyl group” include a silyl group optionally having “1 to 3 substituents selected from a C₁₋₆ alkyl group, a C₂₋₆ alkenyl group, a C₃₋₁₀ cycloalkyl group, a C₆₋₁₄ aryl group and a C₇₋₁₆ aralkyl group, each of which optionally has 1 to 3 substituents selected from Substituent Group A”.

Preferable examples of the optionally substituted silyl group include a tri-C₁₋₆ alkylsilyl group (e.g., trimethylsilyl, tert-butyl(dimethyl)silyl).

In the present specification, examples of the “C₁₋₆ alkylene group” include —CH₂—, —(CH₂)₂—, —(CH₂)₃—, —(CH₂)₄—, —(CH₂)₅—, —(CH₂)₆—, —CH(CH₃)—, —C(CH₃)₂—, —CH(C₂H₅)—, —CH(C₃H₇)—, —CH(CH(CH₃)₂)—, —(CH(CH₃))₂—, —CH₂—CH(CH₃)—, —CH(CH₃)—CH₂—, —CH₂—CH₂—C(CH₃)₂—, —C(CH₃)₂—CH₂—CH₂—, —CH₂—CH₂—CH₂—C(CH₃)₂— and —C(CH₃)₂—CH₂—CH₂—CH₂—.

In the present specification, examples of the “C₂₋₆ alkenylene group” include —CH═CH—, —CH₂—CH═CH—, —CH═CH—CH₂—, —C(CH₃)₂—CH═CH—, —CH═CH—C(CH₃)₂—, —CH₂—CH═CH—CH₂—, —CH₂—CH₂—CH═CH—, —CH═CH—CH₂—CH₂—, —CH═CH—CH═CH—, —CH═CH—CH₂—CH₂—CH₂— and —CH₂—CH₂—CH₂—CH═CH—.

In the present specification, examples of the “C₂₋₆ alkynylene group” include —C≡C—, —CH₂—C≡C—, —C≡C—CH₂—, —C(CH₃)₂—C≡C—, —C≡C—C(CH₃)₂—,—CH₂—C≡C—CH₂—, —CH₂—CH₂—C≡C—, —C≡C—CH₂—CH₂—, —C≡C—C≡C—, —C≡C—CH₂—CH₂—CH₂— and —CH₂—CH₂—CH₂—≡CC—.

In the present specification, examples of the “hydrocarbon ring” include a C₆₋₁₄ aromatic hydrocarbon ring, C₃₋₁₀ cycloalkane and C₃₋₁₀ cycloalkene.

In the present specification, examples of the “C₆₋₄₄ aromatic hydrocarbon ring” include benzene and naphthalene.

In the present specification, examples of the “C₃₋₁₀cycloalkane” include cyclopropane, cyclobutane, cyclopentane, cyclohexane, cycloheptane and cyclooctane.

In the present specification, examples of the “C₃₋₁₀cycloalkene” include cyclopropene, cyclobutene, cyclopentene, cyclohexene, cycloheptene and cyclooctene.

In the present specification, examples of the “heterocycle” include an aromatic heterocycle and a non-aromatic heterocycle, each containing, as a ring-constituting atom besides carbon atom, 1 to 4 hetero atoms selected from a nitrogen atom, a sulfur atom and an oxygen atom.

In the present specification, examples of the “aromatic heterocycle” include a 5- to 14-membered (preferably 5- to 10-membered) aromatic heterocycle containing, as a ring-constituting atom besides carbon atom, 1 to 4 hetero atoms selected from a nitrogen atom, a sulfur atom and an oxygen atom. Preferable examples of the “aromatic heterocycle” include 5- or 6-membered monocyclic aromatic heterocycles such as thiophene, furan, pyrrole, imidazole, pyrazole, thiazole, isothiazole, oxazole, isoxazole, pyridine, pyrazine, pyrimidine, pyridazine, 1,2,4-oxadiazole, 1,3,4-oxadiazole, 1,2,4-thiadiazole, 1,3,4-thiadiazole, triazole, tetrazole, triazine and the like; and

8- to 14-membered fused polycyclic (preferably bi or tricyclic) aromatic heterocycles such as benzothiophene, benzofuran, benzimidazole, benzoxazole, benzisoxazole, benzothiazole, benzisothiazole, benzotriazole, imidazopyridine, thienopyridine, furopyridine, pyrrolopyridine, pyrazolopyridine, oxazolopyridine, thiazolopyridine, imidazopyrazine, imidazopyrimidine, thienopyrimidine, furopyrimidine, pyrrolopyrimidine, pyrazolopyrimidine, oxazolopyrimidine, thiazolopyrimidine, pyrazolopyrimidine, pyrazolotriazine, naphtho[2,3-b]thiophene, phenoxathiine, indole, isoindole, 1H-indazole, purine, isoquinoline, quinoline, phthalazine, naphthyridine, quinoxaline, quinazoline, cinnoline, carbazole, β-carboline, phenanthridine, acridine, phenazine, phenothiazine, phenoxathiine and the like.

In the present specification, examples of the “non-aromatic heterocycle” include a 3- to 14-membered (preferably 4- to 10-membered) non-aromatic heterocycle containing, as a ring-constituting atom besides carbon atom, 1 to 4 hetero atoms selected from a nitrogen atom, a sulfur atom and an oxygen atom. Preferable examples of the “non-aromatic heterocycle” include 3- to 8-membered monocyclic non-aromatic heterocycles such as aziridine, oxirane, thiirane, azetidine, oxetane, thietane, tetrahydrothiophene, tetrahydrofuran, pyrroline, pyrrolidine, imidazoline, imidazolidine, oxazoline, oxazolidine, pyrazoline, pyrazolidine, thiazoline, thiazolidine, tetrahydroisothiazole, tetrahydrooxazole, tetrahydroisoxazole, piperidine, piperazine, tetrahydropyridine, dihydropyridine, dihydrothiopyran, tetrahydropyrimidine, tetrahydropyridazine, dihydropyran, tetrahydropyran, tetrahydrothiopyran, morpholine, thiomorpholine, azepanine, diazepane, azepine, azocane, diazocane, oxepane and the like; and

9- to 14-membered fused polycyclic (preferably bi or tricyclic) non-aromatic heterocycles such as dihydrobenzofuran, dihydrobenzimidazole, dihydrobenzoxazole, dihydrobenzothiazole, dihydrobenzisothiazole, dihydronaphtho[2,3-b]thiophene, tetrahydroisoquinoline, tetrahydroquinoline, 4H-quinolizine, indoline, isoindoline, tetrahydrothieno[2,3-c]pyridine, tetrahydrobenzazepine, tetrahydroquinoxaline, tetrahydrophenanthridine, hexahydrophenothiazine, hexahydrophenoxazine, tetrahydrophthalazine, tetrahydronaphthyridine, tetrahydroquinazoline, tetrahydrocinnoline, tetrahydrocarbazole, tetrahydro-β-carboline, tetrahydroacridine, tetrahydrophenazine, tetrahydrothioxanthene, octahydroisoquinoline and the like.

In the present specification, examples of the “nitrogen-containing heterocycle” include a “heterocycle” containing at least one nitrogen atom as a ring-constituting atom.

In one embodiment, preferable examples of the “non-aromatic heterocyclic group” include a 7- to 14-membered spiro heterocyclic group such as triazaspirononyl (e.g., 1,3,7-triazaspiro[4.4]nonyl), thiadiazaspirononyl (e.g., 7-thia-1,3-diazaspiro[4.4]nonyl), dioxidothiadiazaspirononyl (e.g., 7,7-dioxido-7-thia-1,3-diazaspiro[4.4]nonyl) and the like, in addition to the above-mentioned “3- to 8-membered monocyclic non-aromatic heterocyclic group” and “9- to 14-membered fused polycyclic (preferably bi- or tri-cyclic) non-aromatic heterocyclic group”.

As used herein, “topical composition” refers to a formulation of a compound of the present disclosure and a medium generally accepted in the art for the delivery of the biologically active compound to mammalian skin, e.g., human skin. Such a medium includes all dermatologically acceptable carriers, diluents or excipients therefor.

“Stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposeable mirror images of one another.

“Solvate” refers to a form of a compound complexed by solvent molecules.

“Tautomers” refers to two molecules that are structural isomers that readily interconvert.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

The compounds of the present disclosure, or their pharmaceutically acceptable salts may contain one or more asymmetric centres and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallisation. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

The chemical naming protocol and structure diagrams used herein are a modified form of the I.U.P.A.C. nomenclature system, using the ChemDraw Version 10 software naming program (CambridgeSoft). In chemical structure diagrams, all bonds are identified, except for some carbon atoms, which are assumed to be bonded to sufficient hydrogen atoms to complete the valency.

“Dermatologically acceptable excipient” includes without limitation any adjuvant, carrier, vehicle, excipient, glidant, sweetening agent, diluent, preservative, dye/colorant, flavor enhancer, surfactant, wetting agent, dispersing agent, suspending agent, stabilizer, isotonic agent, solvent, or emulsifier, including those approved by the United States Food and Drug Administration as being acceptable for dermatological use on humans or domestic animals, or which are known, or are suitable for use in dermatological compositions.

“Optional” or “optionally” means that the subsequently described event of circumstances may or may not occur, and that the description includes instances where said event or circumstance occurs and instances in which it does not. When a functional group is described as “optionally substituted,” and in turn, substituents on the functional group are also “optionally substituted” and so on, for the purposes of this disclosure, such iterations are limited to five, preferably such iterations are limited to two.

### Treatment of Dermatological Disorders

In one embodiment, the present disclosure provides for a method [Method 1] for treating a dermatological disorder, the method comprising topically administering to a subject in need thereof a topical composition having a therapeutically effective amount of an IRAK4 inhibitor of Formula I, II (e.g., Compound 1, et seq.), or III (e.g., Compound 2, et seq.); and a dermatologically acceptable excipient.

The present disclosure provides additional embodiments of Method 1 as follows:
1.1 Method 1, wherein the IRAK4 inhibitor is a dual IRAK4/TrkA inhibitor.
1.2 Any of the preceding methods, wherein the IRAK4 inhibitor is a compound according to Compound 1, et seq. or Compound 2, et seq.
1.3 Any of the preceding methods, wherein the dermatological disorder is an inflammatory dermatological disorder.
1.4 The preceding method, wherein the inflammatory dermatological disorder is rosacea, psoriasis, atopic dermatitis, hidradenitis suppurativa, seborrheic dermatitis, contact dermatitis, urticaria, dermatitis herpetiformis, nummular dermatitis, lichen planus, pityriasis rosea, cutaneous lupus, acne, cancers of the skin (e.g. cutaneous T-cell lymphoma), or miliaria.
1.5 Any of Method 1 or 1.3-1.4, wherein the inflammatory dermatological disorder is rosacea.
1.6 The preceding method, wherein the rosacea is papulopustular rosacea.
1.7 Method 1 or 1.1-1.4, wherein the inflammatory dermatological disorder is psoriasis.
1.8 Method 1 or 1.1-1.4, wherein the inflammatory dermatological disorder is atopic dermatitis.
1.9 Method 1 or 1.1-1.4, wherein inflammatory dermatological disorder is hidradenitis suppurativa.
1.10 Method 1 or 1.1-1.4, wherein inflammatory dermatological disorder is cutaneous lupus.
1.11 Method 1 or 1.1-1.4, wherein inflammatory dermatological disorder is acne.
1.12 Method 1 or 1.1-1.4, wherein inflammatory dermatological disorder is a cancer of the skin (e.g., cutaneous T-cell lymphoma).
1.13 Any of the preceding methods, the subject is a human.

Another embodiment provides a method [Method 2] for reducing inflammation in mammalian skin, the method comprising topically administering to the mammalian skin an effective amount of a topical composition including an IRAK4 inhibitor of compound of Formula I, II (e.g., Compound 1, et seq.), or III (e.g., Compound 2, et seq.) to a subject in need thereof.

The present disclosure provides additional embodiments of Method 2 as follows:
2.1 Method 2, wherein the IRAK4 inhibitor is a dual IRAK4/TrkA inhibitor.
2.2 Any of the preceding methods, wherein the IRAK4 inhibitor is a compound according to Compound 1, et seq. or Compound 2, et seq.
2.3 Any of the preceding methods, wherein the subject is suffering from a dermatological disorder.
2.4 Method 2.3, wherein the dermatological disorder is an inflammatory dermatological disorder.
2.5 The preceding method, wherein the inflammatory dermatological disorder is rosacea, psoriasis, atopic dermatitis, hidradenitis suppurativa, seborrheic dermatitis, contact dermatitis, urticaria, dermatitis herpetiformis, nummular dermatitis, lichen planus, pityriasis rosea, cutaneous lupus, acne, cancers of the skin (e.g. cutaneous T-cell lymphoma), or miliaria.
2.6 Any of Methods 2.3-2.5, wherein the inflammatory dermatological disorder is rosacea.
2.7 The preceding method, wherein the rosacea is papulopustular rosacea.
2.8 Any of Methods 2.3-2.5, wherein the inflammatory dermatological disorder is psoriasis.
2.9 Any of Methods 2.3-2.5, wherein the inflammatory dermatological disorder is atopic dermatitis.
2.10 Any of Methods 2.3-2.5, wherein inflammatory dermatological disorder is hidradenitis suppurativa.
2.11 Any of Methods 2.3-2.5, wherein inflammatory dermatological disorder is cutaneous lupus.
2.12 Any of Methods 2.3-2.5, wherein inflammatory dermatological disorder is acne.
2.13 Any of Methods 2.3-2.5, wherein inflammatory dermatological disorder is a cancer of the skin (e.g., cutaneous T-cell lymphoma).
2.14 Any of the preceding methods, the subject is a human.
2.15 Any of the preceding methods, wherein the mammalian skin is human skin.

A further embodiment provides a method [Method 3] of reducing inflammation and vascular dysfunction in mammalian skin, the method comprising topically administering to the mammalian skin a therapeutically effective amount of a topical composition including an IRAK4 inhibitor a compound of Formula I, II (e.g., Compound 1, et seq.), or III (e.g., Compound 2, et seq.) to a subject in need thereof.

The present disclosure provides additional embodiments of Method 3 as follows:
3.1 Method 3, wherein the IRAK4 inhibitor is a dual IRAK4/TrkA inhibitor.
3.2 Any of the preceding methods, wherein the IRAK4 inhibitor is a compound according to Compound 1, et seq. or Compound 2, et seq.
3.3 Any of the preceding methods, wherein the subject is suffering from a dermatological disorder.
3.4 Method 3.3, wherein the dermatological disorder is an inflammatory dermatological disorder.
3.5 The preceding method, wherein the inflammatory dermatological disorder is rosacea, psoriasis, atopic dermatitis, hidradenitis suppurativa, seborrheic dermatitis, contact dermatitis, urticaria, dermatitis herpetiformis, nummular dermatitis, lichen planus, pityriasis rosea, cutaneous lupus, acne, cancers of the skin (e.g. cutaneous T-cell lymphoma), or miliaria.
3.6 Any of Methods 3.3-3.5, wherein the inflammatory dermatological disorder is rosacea.
3.7 The preceding method, wherein the rosacea is papulopustular rosacea.
3.8 Any of Methods 3.3-3.5, wherein the inflammatory dermatological disorder is psoriasis.
3.9 Any of Methods 3.3-3.5, wherein the inflammatory dermatological disorder is atopic dermatitis.
3.10 Any of Methods 3.3-3.5, wherein inflammatory dermatological disorder is hidradenitis suppurativa.
3.11 Any of Methods 3.3-3.5, wherein inflammatory dermatological disorder is cutaneous lupus.
3.12 Any of Methods 3.3-3.5, wherein inflammatory dermatological disorder is acne.
3.13 Any of Methods 3.3-3.5, wherein inflammatory dermatological disorder is a cancer of the skin (e.g., cutaneous T-cell lymphoma).
3.14
3.15 Any of the preceding methods, the subject is a human.
3.16 Any of the preceding methods, wherein the mammalian skin is human skin.

As used herein, “inflammatory dermatological disorder” refers to disorders involving skin inflammation including, for example, rosacea, psoriasis, atopic dermatitis, hidradenitis suppurativa, seborrheic dermatitis, contact dermatitis, urticaria, dermatitis herpetiformis, nummular dermatitis, lichen planus, pityriasis rosea, cutaneous lupus, acne, cancers of the skin (e.g. cutaneous T-cell lymphoma), and miliaria. Skin inflammation is typically characterized by redness/flushing, pain, pustules, sensation of heat, and/or swelling.

“Lesional skin” of a human having rosacea refers to a site on the skin having active rosacea, such as an active site of erythematotelangiectatic rosacea (e.g., with flushing or visible blood vessels), or an active site of papulopustular rosacea (e.g., skin having an active acne-like breakout of swollen red bumps).

“Mammal” or “mammalian” includes humans and both domestic animals such as laboratory animals and household pets, (e.g., cats, dogs, swine, cattle, sheep, goats, horses, rabbits), and non-domestic animals such as wildlife and the like.

“Therapeutically effective amount” refers to that amount of a compound of the present disclosure which, when administered to a mammal, preferably a human, is sufficient to effect treatment of the disease or condition of interest in a mammal, preferably a human, having the disease or condition. The amount of a compound of the present disclosure which constitutes a “therapeutically effective amount” will vary depending on the compound, the disease or condition and its severity, the manner of administration, and the age of the mammal to be treated, but can be determined routinely by one of ordinary skill in the art having regard to his own knowledge and to this disclosure. Preferably, for purposes of this disclosure, a “therapeutically effective amount” is that amount of a compound of present disclosure which is sufficient to inhibit inflammation of the skin.

“Treating” or “treatment”, as used herein, covers the treatment of the disease or condition of interest in a mammal, preferably a human, and includes:

(i) preventing the disease or condition from occurring in the mammal;

(ii) inhibiting the disease or condition in the mammal, i.e., arresting its development;

(iii) relieving the disease or condition in the mammal, i.e., causing regression of the disease or condition; or

(iv) relieving the symptoms of the disease or condition in the mammal, i.e., relieving the symptoms without addressing the underlying disease or condition; or

As used herein, the terms “disease,” “disorder,” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

“Locally reducing inflammation” refers to a decrease or reduction of local inflammation at the site of topical administration of the pharmaceutical composition. Administering a topical composition as described herein may reduce inflammation at the site of the body where the pharmaceutical composition is topically administered. A reduction in local inflammation may be evidenced by decreased redness, decreased swelling, deceased pain or irritation, a decrease in a sensation of heat, and/or decreased expression of one or more inflammation markers such as interleukin-6 (IL-6), C-C motif chemokine ligand 3 (CCL3, or MIP-1alpha).

In the present description, the term “about” means ±20% of the indicated range, value, or structure, unless otherwise indicated.

In some embodiments, the IRAK4 inhibitor of Formula I, II (e.g., Compound 1, et seq.), or III (e.g., Compound 2, et seq.) is present in the topical composition at a concentration of about 0.005% to about 5% by weight, e.g., a concentration of about 0.05% to about 4% by weight.

In certain embodiments, the pharmaceutical compositions described herein further include a dermatologically acceptable excipient. The dermatologically acceptable excipients may be one or more solvents that solubilize and/or stabilize the active ingredient (e.g., IRAK4 inhibitors) contained therein. The dermatologically acceptable excipients may also include skin penetration enhancers, preservatives, viscosity enhancers, pH adjusters, film forming agents and the like. Non-limiting examples of the suitable excipients include water, PEG 200, PEG 400, ethanol, glycerol, Transcutol P (diethylene glycol monoethyl ether), propylene glycol, 1,3-dimethyl-2-imidazolidinone (DMI), sodium metabisulfite, butylated hydroxytoluene (BHT), benzyl alcohol, sodium benzoate, isopropyl myristate, diisopropyl adipate, crodamol OHS (ethylhexyl hydroxystearate), mineral oil, Betadex, TWEEN 20, Brij S20 (polyoxyethylene (20) stearyl ether), silicones (eg. dimethicone, cylcomethicone etc).

More detailed description of certain suitable excipients is described below. As will be appreciated, components of the pharmaceutical formulations described herein can possess multiple functions. For example, a given substance may act as both a viscosity increasing agent and as an emulsifying agent.

The skin (especially stratum corneum) provides a physical barrier to the harmful effects of the external environment. In doing so, it also interferes with the absorption or transdermal delivery of topical therapeutic drugs. Thus, a suitable dermatologically acceptable excipient may include one or more penetration enhancers (or permeation enhancers), which are substances that promote the diffusion of the therapeutic drugs (e.g., the IRAK4 inhibitors described herein) through the skin barrier. They typically act to reduce the impedance or resistance of the skin to allow improved permeation of the therapeutic drugs. In particular, substances which would perturb the normal structure of the stratum corneum are capable of disrupting the intercellular lipid organization, thus reducing its effectiveness as a barrier. These substances could include any lipid material which would partition into the stratum corneum lipids causing a direct effect or any material which would affect the proteins and cause an indirect perturbation of the lipid structure. Furthermore, solvents, such as ethanol, can remove lipids from the stratum corneum, thus destroying its lipid organization and disrupting its barrier function.

Examples of penetration enhancers or barrier function disrupters include, but are not limited to, alcohol-based enhancers, such as alkanols with one to sixteen carbons, benzyl alcohol, butylene glycol, diethylene glycol, glycofurol, glycerides, glycerin, glycerol, phenethyl alcohol, polypropylene glycol, polyvinyl alcohol, and phenol; amide-based enhancers, such as N-butyl-N-dodecylacetamide, crotamiton, N,N-dimethylformamide, N,N-dimethylacetamide, N-methyl formamide, and urea; amino acids, such as L-α-amino acids and water soluble proteins; azone and azone-like compounds, such as azacycloalkanes; essential oils, such as almond oil, amyl butyrate, apricot kernel oil, avocado oil, camphor, castor oil, 1-carvone, coconut oil, corn oil, cotton seed oil, eugenol, menthol, oil of anise, oil of clove, orange oil, peanut oil, peppermint oil, rose oil, safflower oil, sesame oil, shark liver oil (squalene), soybean oil, sunflower oil, and walnut oil; vitamins and herbs, such as aloe, allantoin, black walnut extract, chamomile extract, panthenol, papain, tocopherol, and vitamin A palmitate; waxes, such as candelilla wax, carnauba wax, ceresin wax, beeswax, lanolin wax, jojoba oil, petrolatum; mixes, such as primary esters of fractionated vegetable oil fatty acids with glycerine or propylene glycol, and interesterified medium chain triglyceride oils; fatty acids and fatty acid esters, such as amyl caproate, butyl acetate, caprylic acid, cetyl ester, diethyl sebacate, dioctyl malate, elaidic acid ethyl caprylate, ethyl glycol palmitostearate, glyceryl beheate, glucose glutamate, isobutyl acetate, laureth-4, lauric acid, malic acid, methyl caprate, mineral oil, myristic acid, oleic acid, palmitic acid, PEG fatty esters, polyoxylene sorbitan monooleate, polypropylene glycols, propylene glycols, saccharose disterate, salicylic acid, sodium citrate, stearic acid, soaps, and caproic-, caprylic-, capric-, and lauric-triglycerides; macrocylics, such as butylated hydroxyanisole, cyclopentadecanolide, cyclodextrins; phospholipid and phosphate enhancers, such as dialkylphosphates, ditetradecyl phosphate, lecithin, 2-pyrrolidone derivatives, such as alkyl pyrrolidone-5-carboxylate esters, pyroglutamic acid esters, N-methyl pyrrolidone, biodegradable soft penetration enhancers, such as dioxane derivatives and dioxolane derivatives; sulphoxide enhancers, such as dimethyl sulphoxide and decylmethyl sulphoxide; acid enhancers, such as alginic acid, sorbic acid, and succinic acid; cyclic amines; imidazolinones; imidazoles; ketones, such as acetone, dimethicone, methyl ethyl ketone, and pentanedione; lanolin derivatives, such as lanolin alcohol, PEG 16 lanolin, and acetylated lanolin; oxazolines; oxazolindinones; proline esters; pyrroles, urethanes; and surfactants, such as nonoxynols, polysorbates, polyoxylene alcohols, polyoxylene fatty acid esters, sodium lauryl sulfate, and sorbitan monostearate.

The topical compositions described herein typically contain one or more carriers, which preferably have a vapor pressure greater than or equal to 23.8 mm Hg at 25° C. Preferred concentration range of a single carrier or the total of a combination of carriers can be from about 0.1 wt. % to about 10 wt. %, more preferably from about 10 wt. % to about 50 wt. %, more specifically from about 50 wt. % to about 95 wt. % of the dermatological composition. Non-limiting examples of the solvent include water (e.g., deionized water) and lower alcohols, including ethanol, 2-propanol and n-propanol.

A dermatological composition of the present disclosure can contain one or more hydrophilic co-solvents, which are miscible with water and/or lower chain alcohols and preferably have a vapor pressure less than water at 25° C. (˜23.8 mm Hg). The carrier typically has a vapor pressure greater than or equal to the hydrophilic co-solvent as to concentrate the compound of Formula I, II (e.g., Compound 1, et seq.), or III (e.g., Compound 2, et seq.) on the skin. A hydrophilic co-solvent may be a glycol, specifically propylene glycol. In particular, the propylene glycol can be from the class of polyethylene glycols, specifically polyethylene glycols ranging in molecular weight from 200 to 20000. Preferably, the solvent would be part of a class of glycol ethers. More specifically, a hydrophilic co-solvent of the present disclosure would be diethylene glycol monoethyl ether (transcutol). As used herein, “diethylene glycol monoethyl ether” (“DGME”) or “transcutol” refers to 2-(2-ethoxyethoxy)ethanol {CAS NO 001893} or ethyoxydiglycol. Another preferred co-solvent is 1,3-dimethyl-2-imidazolidinone (DMI).

The topical compositions described herein may also contain one or more “humectant(s)” used to provide a moistening effect. Preferably the humectant remains stable in the composition. Any suitable concentration of a single humectant or a combination of humectants can be employed, provided that the resulting concentration provides the desired moistening effect. Typically, the suitable amount of humectant will depend upon the specific humectant or humectants employed. Preferred concentration range of a single humectant or the total of a combination of humectants can be from about 0.1 wt. % to about 70 wt. %, more preferably from about 5.0 wt. % to about 30 wt. %, more specifically from about 10 wt. % to about 25 wt. % of the dermatological composition. Non-limiting examples for use herein include glycerin, polyhydric alcohols and silicone oils. More preferably, the humectant is glycerin, propylene glycol and/or cyclomethicone. Specifically, the filler would be glycerine and/or cyclomethicone.

In certain embodiments, the pharmaceutical compositions include a viscosity enhancing agent or an emulsifier. Gelling agents are used to increase the viscosity of the final composition. Emulsifiers are substances that stabilize an emulsion. The viscosity increasing agent can also act as an emulsifying agent. Typically, the concentration and combination of viscosity increasing agents will depend on the physical stability of the finished product. Preferred concentration range of a viscosity increasing agent can be from about 0.01 wt. % to about 20 wt. %, more preferably from about 0.1 wt. % to about 10 wt. %, more specifically from about 0.5 wt. % to about 5 wt. % of the dermatological composition. Non-limiting examples of viscosity increasing agents for use herein include classes of celluloses, acrylate polymers and acrylate crosspolymers, such as, hydroxypropyl cellulose, hydroxymethyl cellulose, Pluronic PF127 polymer, carbomer 980, carbomer 1342 and carbomer 940, more preferably hydroxypropyl cellulose, Pluronic PF127 carbomer 980 and carbomer 1342, more specifically hydroxypropyl cellulose (Klucel® EF, GF and/or HF), Pluronic PF127, carbomer 980 and/or carbomer 1342 (Pemulen® TR-1, TR-2 and/or Carbopol® ETD 2020). Examples of emulsifiers for use herein include polysorbates, laureth-4, and potassium cetyl sulfate.

The topical compositions described herein may contain one or more anti-oxidants, radical scavengers, and/or stabilizing agents, preferred concentration range from about 0.001 wt. % to about 0.1 wt. %, more preferably from about 0.1 wt. % to about 5 wt. % of the dermatological composition. Non-limiting examples for use herein include butylatedhydroxytoluene, butylatedhydroxyanisole, ascorbyl palmitate, citric acid, vitamin E, vitamin E acetate, vitamin E-TPGS, ascorbic acid, tocophersolan and propyl gallate. More specifically the anti-oxidant can be ascorbyl palmitate, vitamin E acetate, vitamin E-TPGS, vitamin E or butylatedhydroxy toluene.

The topical compositions described herein may also contain preservatives that exhibit anti-bacterial and/or anti-fungal properties. Preservatives can be present in a gelled dermatological composition of the present disclosure to minimize bacterial and/or fungal over its shelf-life. Preferred concentration range of preservatives in a dermatological composition of the present disclosure can be from about 0.001 wt. % to about 0.01 wt. %, more preferably from about 0.01 wt. % to about 0.5 wt. % of the dermatological composition. Non-limiting examples for use herein include diazolidinyl urea, methylparaben, propylparaben, tetrasodium EDTA, and ethylparaben. More specifically the preservative would be a combination of methylparaben and propylparaben.

The topical compositions described herein may optionally include one or more chelating agents. As used herein, the term “chelating agent” or “chelator” refers to those skin benefit agents capable of removing a metal ion from a system by forming a complex so that the metal ion cannot readily participate in or catalyze chemical reactions. The chelating agents for use herein are preferably formulated at concentrations ranging from about 0.001 wt. % to about 10 wt. %, more preferably from about 0.05 wt. % to about 5.0 wt. % of the dermatological composition. Non-limiting examples for use herein include EDTA, disodium edeate, dipotassium edeate, cyclodextrin, trisodium edetate, tetrasodium edetate, citric acid, sodium citrate, gluconic acid and potassium gluconate. Specifically, the chelating agent can be EDTA, disodium edeate, dipotassium edate, trisodium edetate or potassium gluconate.

The topical compositions described herein may include one or more compatible cosmetically acceptable adjuvants commonly used, such as colorants, fragrances, emollients, and the like, as well as botanicals, such as aloe, chamomile, witch hazel and the like.

Alternatively, other pharmaceutical delivery systems may be employed for the pharmaceutical compositions of the present disclosure. Liposomes and emulsions are well-known examples of delivery vehicles that may be used to deliver active compound(s) or prodrug(s). Certain organic solvents such as dimethylsulfoxide (DMSO) may also be employed.

The topical compositions described herein may be provided in any cosmetically suitable form, preferably as a lotion, a cream, or a ointment, as well as a sprayable liquid form (e.g., a spray that includes the IRAK4 inhibitor in a base, vehicle or carrier that dries in a cosmetically acceptable way without the greasy appearance that a lotion or ointment would have when applied to the skin).

Any suitable amount of a compound of Formula I, II (e.g., Compound 1, et seq.), or III (e.g., Compound 2, et seq.) can be employed in such dermatological compositions, provided the amount effectively reduces local inflammation and/or vascular dysfunction, and remains stable in the composition over a prolonged period of time. Preferably, the stability is over a prolonged period of time, e.g., up to about 3 years, up to 1 year, or up to about 6 months, which is typical in the manufacturing, packaging, shipping and/or storage of dermatologically acceptable compositions. A compound of Formula I, II (e.g., Compound 1, et seq.), or III (e.g., Compound 2, et seq.) can be in solution, partially in solution with an undissolved portion or completely undissolved suspension. A compound of Formula I, II (e.g., Compound 1, et seq.), or III (e.g., Compound 2, et seq.) can be present in a dermatological composition of the present disclosure in a concentration range from about 0.001 wt. % to about 80 wt. %, from about 0.001 wt. % to about 50 wt. %, from about 0.001 wt. % to about 25 wt. %, or from about 0.001 wt. % to about 6 wt. % of the dermatological composition. In one embodiment, a compound of Formula I, II (e.g., Compound 1, et seq.), or III (e.g., Compound 2, et seq.) can be present in a concentration range of from about 0.001 wt. % to about 10 wt. %, from about 0.1 wt. % to about 10 wt. % or from about 1.0 wt. % to about 5.0 wt. % of the dermatological composition.

In treating the inflammatory dermatological disorders such as rosacea, the topical composition comprising a compound of Formula I, II (e.g., Compound 1, et seq.), or III (e.g., Compound 2, et seq.) is preferably administered directly to the affected area of the skin (e.g., rosacea lesion) of the human in need thereof. When such compositions are in use (e.g., when a dermatological composition comprising a compound of Formula I, II (e.g., Compound 1, et seq.), or III (e.g., Compound 2, et seq.) and a dermatologically acceptable excipient is placed upon the skin of the human in need thereof), the compound of Formula I, II (e.g., Compound 1, et seq.), or III (e.g., Compound 2, et seq.) is in continuous contact with the skin of the patient, thereby effecting penetration and treatment.

In topically administering the pharmaceutical compositions of the present disclosure, the skin of the human to be treated can be optionally pre-treated (such as washing the skin with soap and water or cleansing the skin with an alcohol-based cleanser) prior to administration of the dermatological composition of the present disclosure.

The pharmaceutical compositions of the present disclosure may, if desired, be presented in a pack or dispenser device which may contain one or more unit dosage forms containing the active compound(s). The topical composition described herein may also be provided in a patch with the topical composition on the side of the patch that directly contacts the skin. Dermatologically acceptable adhesives may be used to affix the patch to the skin for an extended period of time.

The following Examples may be used by one skilled in the art to determine the effectiveness of the compounds of the present disclosure in treating a human having a dermatological condition characterized by inflammation.

### EXAMPLES

### Example 1

### Topical Screening of Compounds for Reduction of Inflammation

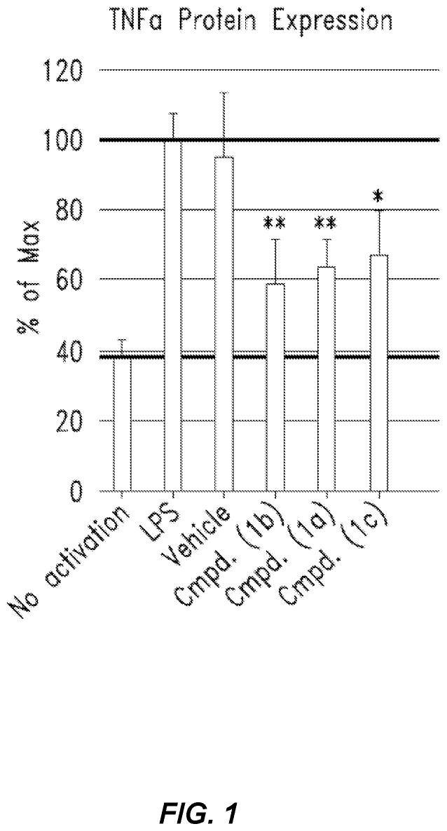

A skin Resident Immune Cell Assay (sRICA) was used to test compounds for reduction of inflammation. In this model, human surgical skin waste was cultured in a transwell system, with the dermis in contact with cell culture media and the stratum corneum exposed to air. To perform the assay, each human skin sample was defatted and dermatomed to 750 μm. Next, 8 mm punch biopsies were obtained and placed in a membrane transwell. The transwells were inserted into culture wells with complete media, and a cocktail of cytokines and antibodies were added to promote skin resident immune cell polarization. The transwells were treated with LPS as a positive control, a vehicle as a negative control, and various dual IRAK/TrkA inhibitors. TNFα protein expression was measured and the mean TNFa protein level in the LPS treated samples was set to 100%. The results for compounds of Formulae (Ia), (Ib), and (Ic) are shown in FIG. 1.

Overall results for all tested compounds are summarized below in Table 1. Results are expressed as a percentage of TNFα expression relative to the LPS treated sample. The transwell treated only with the vehicle showed 95.24% TNFα expression, while the untreated transwell showed 38.14% TNFα expression.

TABLE 1
Results for Select Compounds in Comparison with Controls

| | TNFα Protein |
|---|---|
| | Expression (% relative |
| Compound | to positive control) |
| 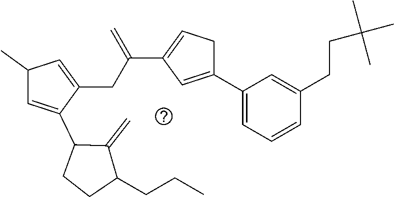 [SMILES: C=C(CC1=CC(C)C=C1C1CCC(CCC)C1=C)C1=CCC(c2cccc(CCC(C)(C)C)c2)=C1] | 64.03 |
| 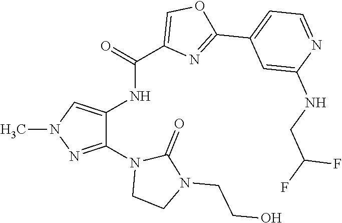 [SMILES: Cn1cc(NC(=O)c2coc(-c3ccnc(NCC(F)F)c3)n2)c(N2CCN(CCO)C2=O)n1] | 72.75 |
| 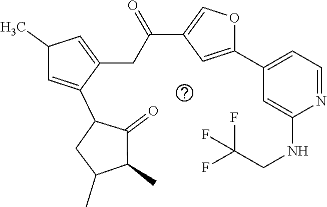 [SMILES: CC1C=C(CC(=O)c2coc(-c3ccnc(NCC(F)(F)F)c3)c2)C(C2CC(C)[C@H](C)C2=O)=C1] | 67.49 |
| 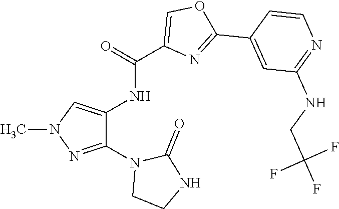 [SMILES: Cn1cc(NC(=O)c2coc(-c3ccnc(NCC(F)(F)F)c3)n2)c(N2CCNC2=O)n1] | 59.13 |
| 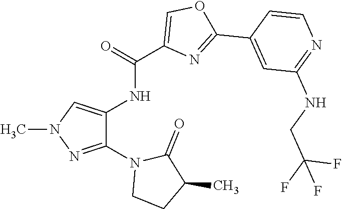 [SMILES: C[C@H]1CCN(c2nn(C)cc2NC(=O)c2coc(-c3ccnc(NCC(F)(F)F)c3)n2)C1=O] | 92.45 |
| 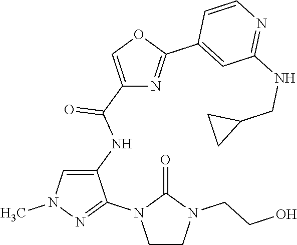 [SMILES: Cn1cc(NC(=O)c2coc(-c3ccnc(NCC4CC4)c3)n2)c(N2CCN(CCO)C2=O)n1] | 74.41 |
| 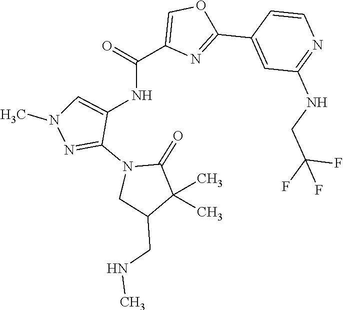 [SMILES: CNCC1CN(c2nn(C)cc2NC(=O)c2coc(-c3ccnc(NCC(F)(F)F)c3)n2)C(=O)C1(C)C] | 92.45 |
| 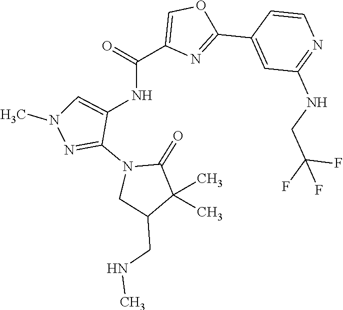 [SMILES: CNCC1CN(c2nn(C)cc2NC(=O)c2coc(-c3ccnc(NCC(F)(F)F)c3)n2)C(=O)C1(C)C] | 93.64 |
| 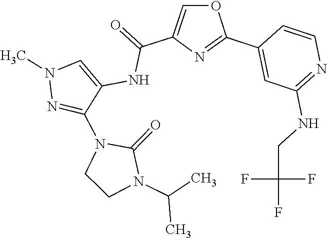 [SMILES: CC(C)N1CCN(c2nn(C)cc2NC(=O)c2coc(-c3ccnc(NCC(F)(F)F)c3)n2)C1=O] | 90.44 |
| 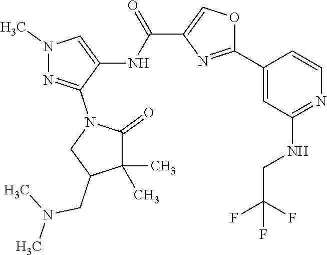 [SMILES: CN(C)CC1CN(c2nn(C)cc2NC(=O)c2coc(-c3ccnc(NCC(F)(F)F)c3)n2)C(=O)C1(C)C] | 86.94 |
| 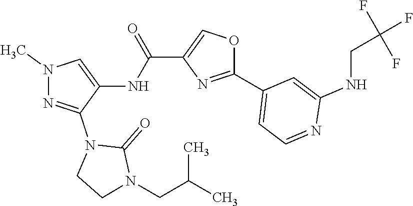 [SMILES: CC(C)CN1CCN(c2nn(C)cc2NC(=O)c2coc(-c3ccnc(NCC(F)(F)F)c3)n2)C1=O] | 89.24 |
| 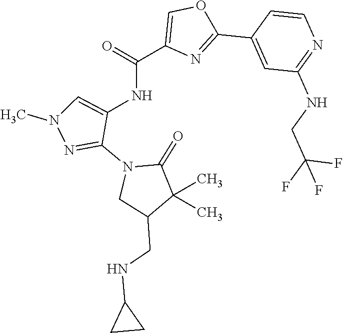 [SMILES: Cn1cc(NC(=O)c2coc(-c3ccnc(NCC(F)(F)F)c3)n2)c(N2CC(CNC3CC3)C(C)(C)C2=O)n1] | 72.95 |
| 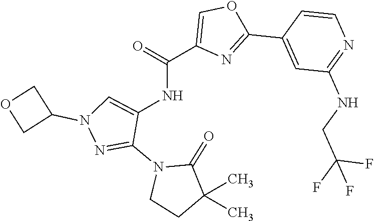 [SMILES: CC1(C)CCN(c2nn(C3COC3)cc2NC(=O)c2coc(-c3ccnc(NCC(F)(F)F)c3)n2)C1=O] | 97.84 |

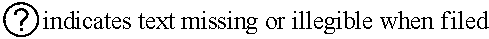

Further studies were carried out with a representative compound selected from Table 1 (Compound 1) above for the efficacy of the compound to mediate innate inflammatory response Immune cells of the monocyte and macrophage lineage are well known to express an array of different TLRs, but many other cells types also express toll-like receptors (TLR), such as keratinocytes, fibroblasts, and other stromal cells. TLRs can be expressed either at the cell surface or endosomally, depending on the typical location of Danger Associated Molecular Patterns or Pathogen Associated Molecular Patterns (i.e. DAMPs or PAMPs) they detect. Activation of TLRs results in cytokine and chemokine production and elicitation of an inflammatory response. All TLRs (except for TLR3) are partially dependent IRAK4 for signaling.

The sRICA model was utilized, and skin samples were treated with Compound 1 overnight prior to stimulation with PamCSK (TLR1/2 agonist), LPS (TLR4 agonist) or Flagellin (TLR5 agonist). The IC50 for various downstream inflammatory biomarkers were recorded. The results are summarized below in Table 2.

TABLE 2
Anti-inflammatory efficacy of Compound
1 following treatment with TLR agonists

| Stimulant | Output analyte | IC50 | |
|---|---|---|---|
| PamCSK | GM-CSF | <50 | nM |
| (TLR1/2 agonist) | IL-10 | <50 | nM |
| | MIP-1a | <100 | nM |
| | TNFa | <50 | nM |
| LPS | GM-CSF | <50 | nM |
| (TLR4 agonist) | IL-10 | <500 | nM |
| | MIP-1a | <500 | nM |
| | TNFa | <50 | nM |
| Flagellin | GM-CSF | <100 | nM |
| (TLR5 agonist) | IL-10 | <5 | uM |
| | MIP-1a | <500 | nM |
| | TNFa | <500 | nM |

Similar tests were carried out for skin samples were treated with IL-1β (which utilizes IRAK4 for signaling downstream of the IL-1 receptor). Compound 1 was added to skin overnight prior to being stimulated with IL-1β and IC50 for various downstream inflammatory biomarkers were recorded. The results are summarized below in Table 3.

TABLE 3
Anti-inflammatory efficacy of select compound
following treatment with IL-1β

| Stimulant | Output Analyte | IC50 | |
|---|---|---|---|
| IL-1β | GM-CSF | <100 | nM |
| | IL-10 | <50 | nM |
| | MIP-1a | <100 | nM |
| | TNFa | <50 | nM |

Finally, skin samples were treated with Compound 1 overnight prior to stimulation with a mixture of cytokines and antibodies to mimic T-helper 2 and T-helper 17 inflammatory responses. The IC50 for various downstream inflammatory biomarkers were recorded. The results are summarized below in Table 4.

TABLE 4
Anti-inflammatory efficacy of select compound following
simulated T-helper cell inflammatory response

| T-helper cell response | Output Analyte | IC50 | |
|---|---|---|---|
| Th2 sRICA | TARC | <500 | nM |
| | GM-CSF | <500 | nM |
| | IL-5 | <50 | nM |
| Th17 sRICA | TNFa | <50 | nM |
| | IL-8 | <500 | nM |
| | IP-10 | <5 | uM |
| | CXCL9 | <5 | uM |

### Example 2

### Expression Comparison for Non-Lesional Versus Lesional Rosacea

Five patients with mild-moderate, untreated, papulopustular rosacea (PPR) were enrolled and completed the study. Each patient donated 3 punch biopsies: 1 non-lesional (NL) and 2 lesional (LS) papules. Each biopsy was bisected, resulting in a total of 6 biopsy pieces. NL tissue was left untreated. LS tissue was either left untreated (vehicle), treated with a dual IRAK/TrkA inhibitor according to the present disclosure (1 uM or 500 uM) or clobetasol (1 uM). Tissue was cultured in media at 37 C, 5% CO₂ for 24 hours. After culture, the tissue was submerged in RNAlater for subsequent RNA isolation and RNA seq, and conditioned cell media was collected into 2×500 ul aliquots for proteomic analysis.

Proteomic analysis included use of the OLink platform. OLink proteomics, includes immunoassay, extension, preamplification, and detection by microfluidic qPCR, and has a high multiplex ability, but not absolute quantification of protein (only relative expression).

For the OLink proteomics platform, 40 μl of conditioned cell media was sent to OLINK proteomics for evaluation on their human Inflammation Panel (92 proteins):

IL8; VEGFA; CD8A; MCP-3; GDNF; CDCP1; CD244; IL7; OPG; LAP TGF-β1; uPA; IL6; IL-17C; MCP-1; IL-17A; CXCL11; AXIN1; TRAIL; IL-20RA; CXCL9; CST5; IL-2RB; IL-1 alpha; OSM; IL2; CXCL1; TSLP; CCL4; CD6; SCF; IL18; SLAMF1; TGF-alpha; MCP-4; CCL11; TNFSF14; FGF-23; IL-10RA; FGF-5; MMP-1; LIF-R; FGF-21; CCL19; IL-15RA; IL-10RB; IL-22 RA1; IL-18R1; PD-L1; β-NGF; CXCL5; TRANCE; HGF; IL-12B; IL-24; IL13; ARTN; MMP-10; IL10; TNF; CCL23; CD5; CCL3; Flt3L; CXCL6; CXCL10; 4E-BP1; IL-20; SIRT2; CCL28; DNER; EN-RAGE; CD40; IL33; IFN-gamma; FGF-19; IL4; LIF; NRTN; MCP-2; CASP-8; CCL25; CX3CL1; TNFRSF9; NT-3; TWEAK; CCL20; ST1A1; STAMBP; ILS; ADA; TNFB; and CSF-1.

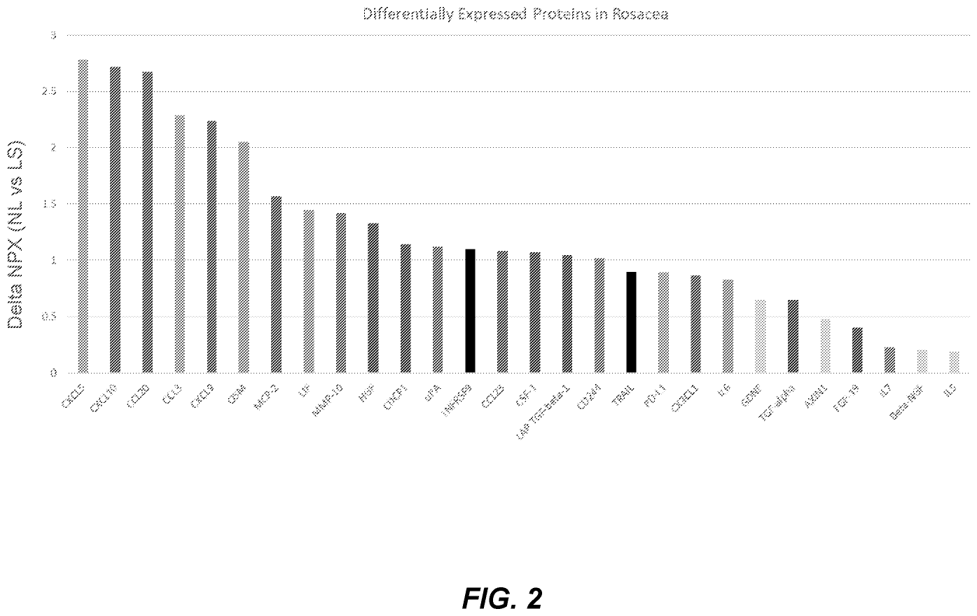

Normalized protein expression (NPX) was calculated for expression of each gene in the NL and LS samples, and the difference in expression of each gene in NL versus LS was calculated (as delta NPX). As shown in FIG. 2, the OLink method detected 28 statistically significant protein expression differences (p<0.05 (paired, 2-sided t-test)) between LS and NS samples. Proteins that had more than two-fold higher expression in LS versus NL were CXCL5, CXCL10, CCL20, CCL3, CXCL9, and OSM. Examples of T helper markers with statistically significant upregulation in LS (compared to NS) include CXCL9, CXCL10, IL-5, and CCL20. TNF pathway proteins with statistically significant upregulation in LS (compared to NS) include TRAIL, and TNFRSF. IL-6 pathway proteins upregulated in LS (compared to NS) include IL-6, LIF, and OSM. Additionally, modest expression of Th1, Th2, and Th17 markers in LS samples indicates adaptive inflammation is also present in this disease. Furthermore, the neurogenic marker β-NGF has statistically significant upregulation in LS versus NL samples.

Next, the protein expression results obtained using the OLink platform were verified using MesoScale Discovery (MSD). MSD involves coating of carbon electrodes with multiple capture antibodies each coated onto discrete spots in the same plate, and is a multiplex platform that allows for absolute quantification of protein expression, as measured in pg/mL. For MSD quantification, 150 μl of conditioned cell media was analyzed using MSD platform using the human Cytokine 30-plex kit:

In summary, this was the first quantitative proteomic assessment of papulopustular rosacea using two different proteomic platforms. The results align with the known/proposed pathology of rosacea, and demonstrate heterogeneous inflammation, with a strong innate immune signature and evidence of neuro-vasculature involvement. This study also identifies several proteins not previously described to be upregulated in PPR: e.g., OSM, uPA, and CXCL5. These results suggest that potent inhibitors of IRAK4 and TrkA would be useful for the treatment of inflammatory skin diseases, such as rosacea.

### Example 3

### Expression Comparison for Non-Lesional (+/−IL-1β) Versus Lesional

Rosacea

IL-1 family cytokines, such as IL-1β, have been described to be upregulated in rosacea, and IL-1β is an important mediator of the inflammatory response Thus, it was hypothesized that IL-1β may be an upstream activator of the inflammation related proteins expressed in lesional rosacea.

Skin samples were obtained as described in Example 2. The NL samples were treated with IL-1β and the protein expression profile was compared to LS samples, as determined using OLink. The results showed that 30% of the rosacea LS proteome can be induced in NL samples by exposure to IL-1β. In particular CCL20, uPA, LIF, IL-6, OSM, and CCL3 all had statistically significant increased expression in NL samples treated with IL-1β, as compared to untreated samples, which is consistent with the statistically significant increased expression of each of these proteins in LS samples as compared to untreated NL samples.

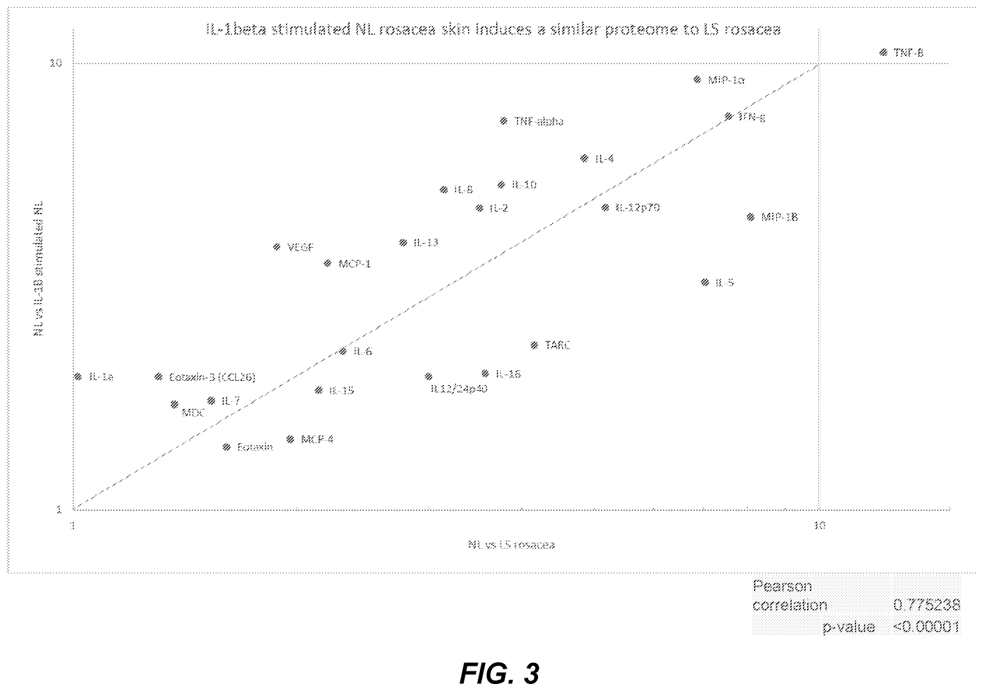

To confirm the results achieved with OLink, the experiment was repeated using another protein analysis technique, the MSD protein multiplex assay. For 27 of the 30 proteins analyzed, the results showed a strong correlation between the differential expression of 1) NL versus IL-1β treated NL and 2) NL versus LS samples. A graph plotting the correlation for the 27 proteins is shown in FIG. 3.

In summary, incubation of non-lesional rosacea skin with IL-β can induce a proteomic signature similar to lesional rosacea skin. These results demonstrate a correlative role of IL-1β in rosacea inflammation, suggesting again that potent inhibitors of IRAK4 and TrkA would be useful for the treatment of inflammatory skin diseases, such as rosacea.

### Example 4

### Protein Expression Following Treatment with a Compound of Formula (I)

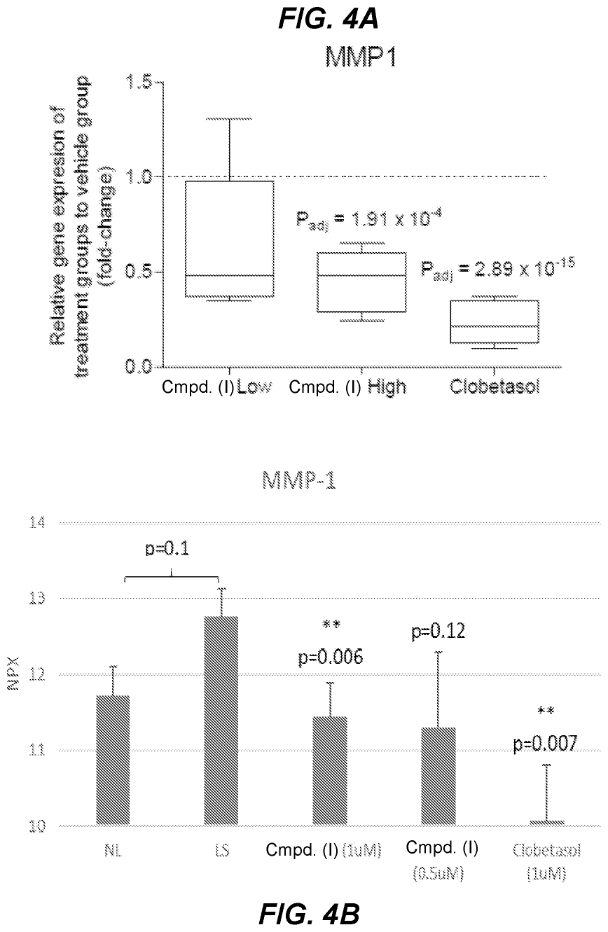

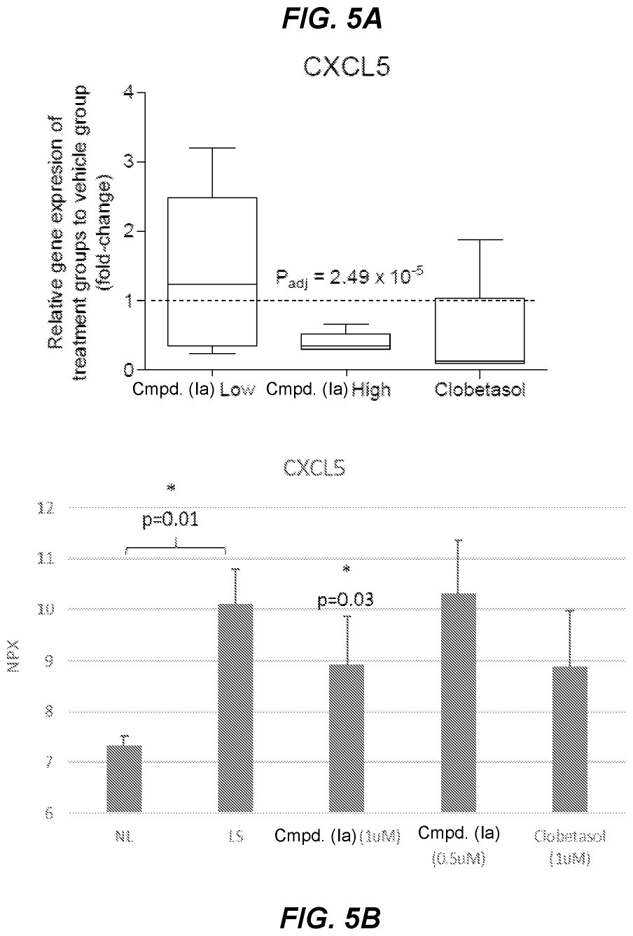

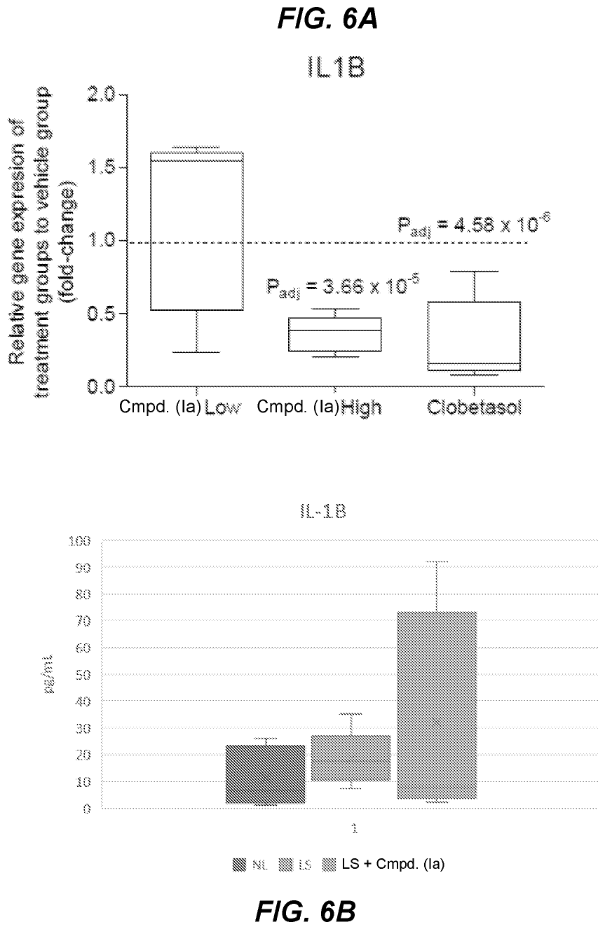

RNAseq was used to determine if genes upregulated in LS compared to NS samples are downregulated in LS samples following treatment with a compound of Formula (Ia). The RNAseq results showed that MMP-1, CXCL5, and IL-1β significantly downregulated by treatment with the compound of Formula (Ia). MMP-1 and CXCL5 were also shown to be downregulated by the compound of Formula (Ia) using the OLink proteomics platform. MMP-1 results are shown in FIG. 4A (RNAseq) and FIG. 4B (OLink); CXCL5 results are shown in FIG. 5A (RNAseq) and FIG. 5B (OLink); and IL-1(3 results are shown in FIG. 6A (RNAseq) and FIG. 6B (OLink).

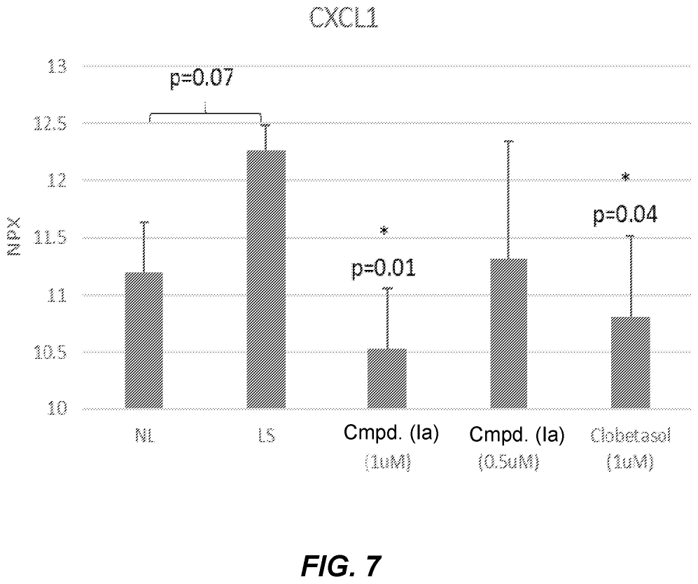

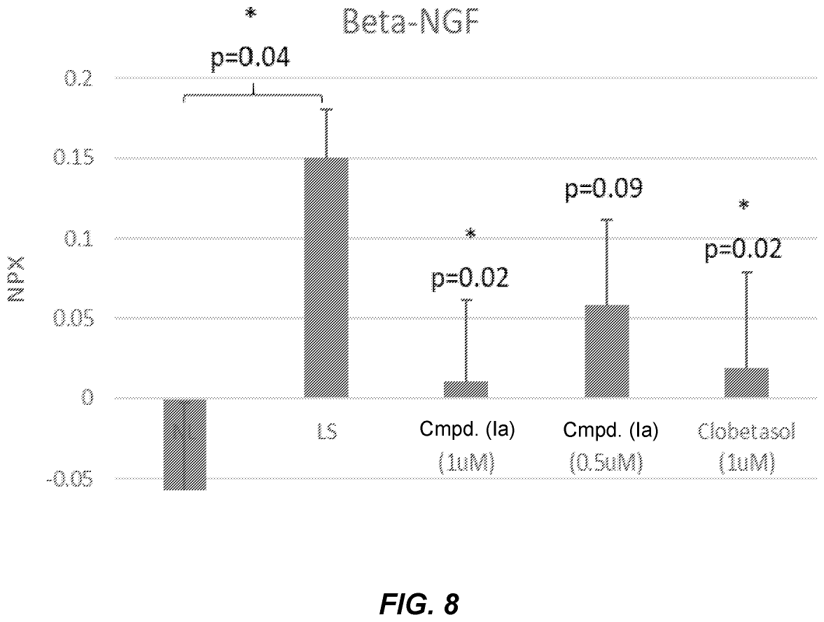

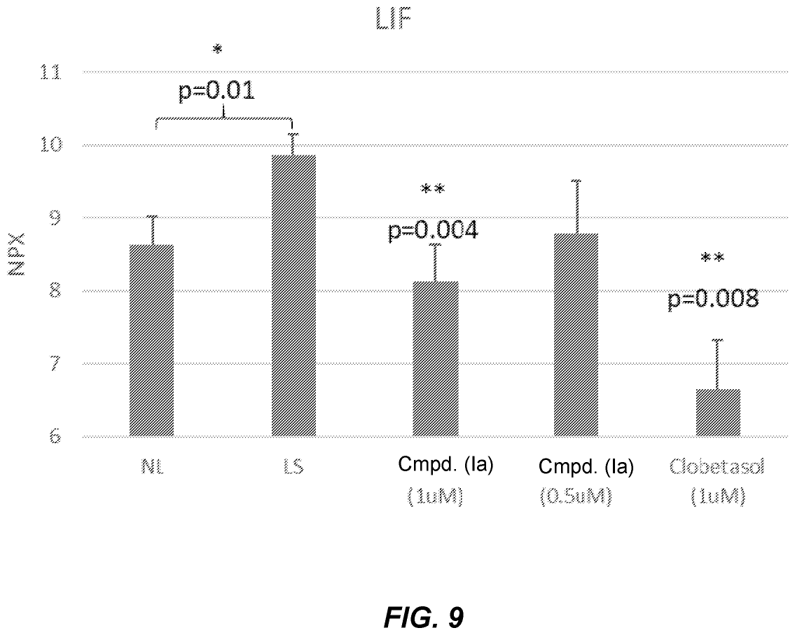

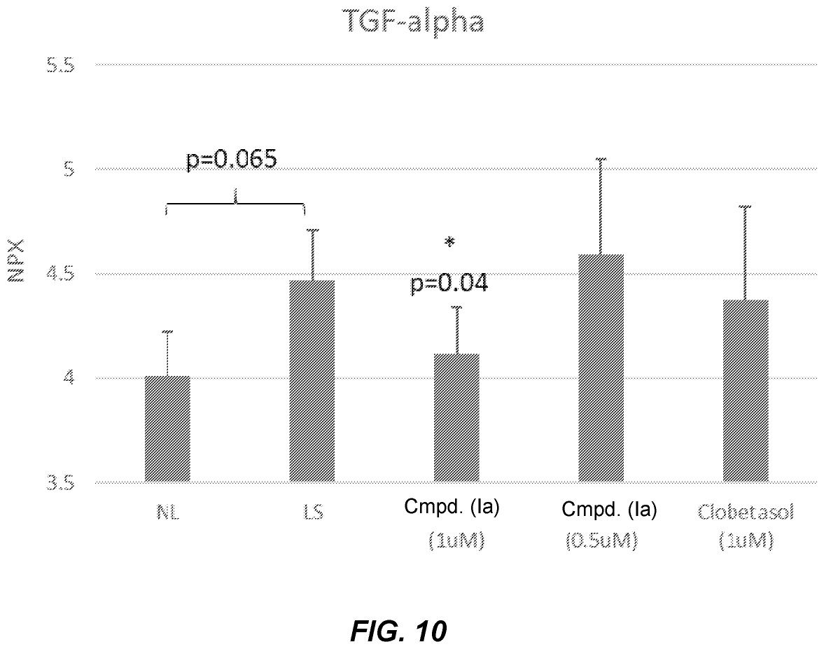

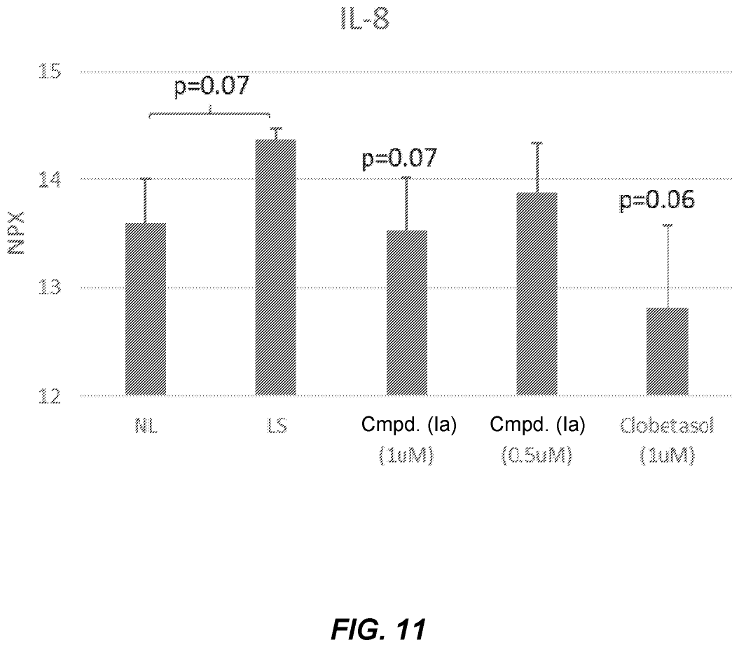

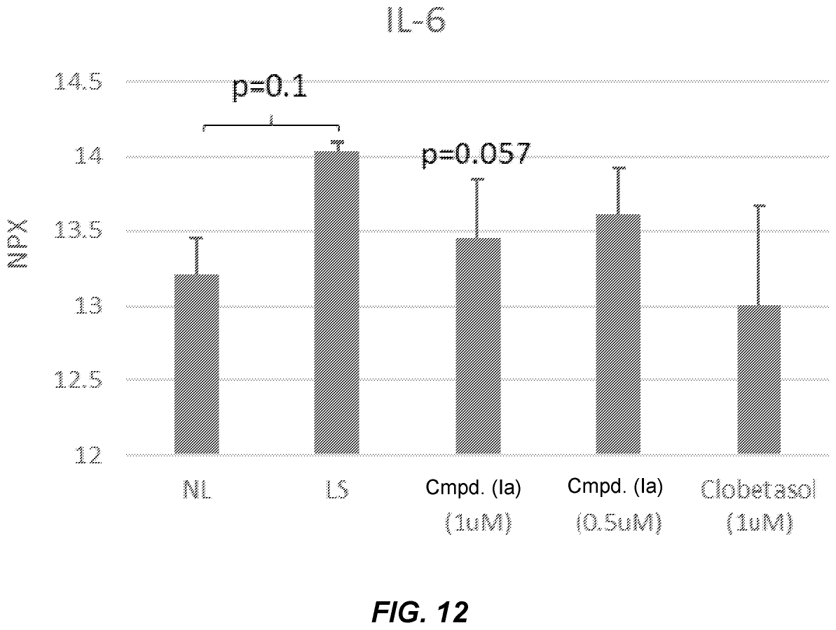

Using the OLink platform, several other proteins were shown to have a statistically significant decrease in expression 24 hours following treatment with the compound of Formula (Ia). Protein expression was measured from NL samples, untreated LS samples, and LS samples treated with the compound of Formula (Ia) at 1 μM, the compound of Formula (Ia) at 0.5 μM, or clobetasol at 1 μM. FIG. 7 shows protein expression results for CXCL1; FIG. 8 shows protein expression results for β NGF; FIG. 9 shows protein expression results for LIF; FIG. 10 shows protein expression results for TGF-alpha; FIG. 11 shows protein expression results for IL-8; and FIG. 12 shows protein expression results for IL-6. CXCL1, β-NGF, LIF, TGF-alpha, IL-8, and IL-6 are all proteins that have reduced expression in NL compared to LS samples. Additionally, CXCL6, IL-24, MCP-1, and NRTN had decreased expression following treatment with the compound of Formula (Ia). However, these proteins do not have a statistically significant different between NL and LS samples, and thus may play a role in rosacea pathology and suggests that NL skin also has underlying pathology.

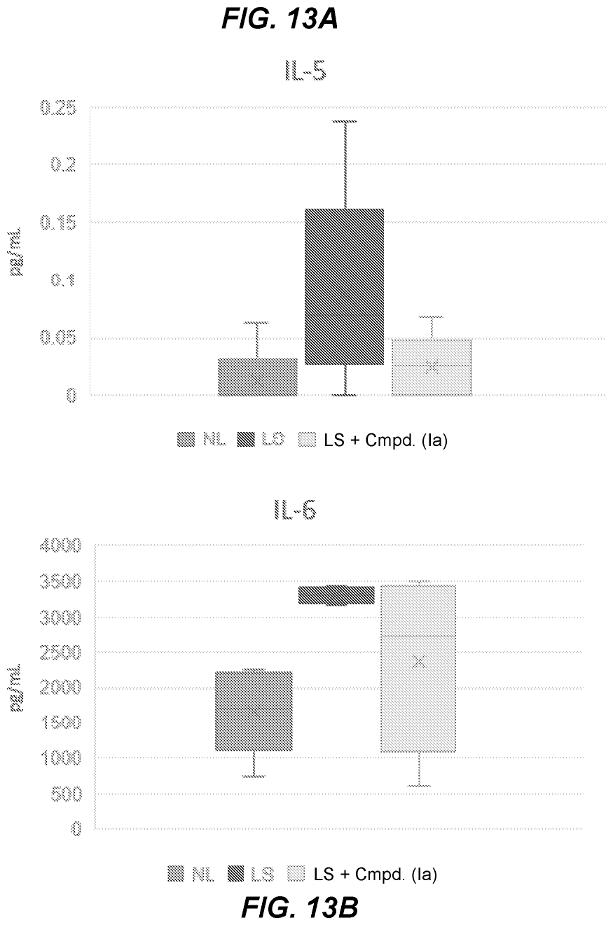

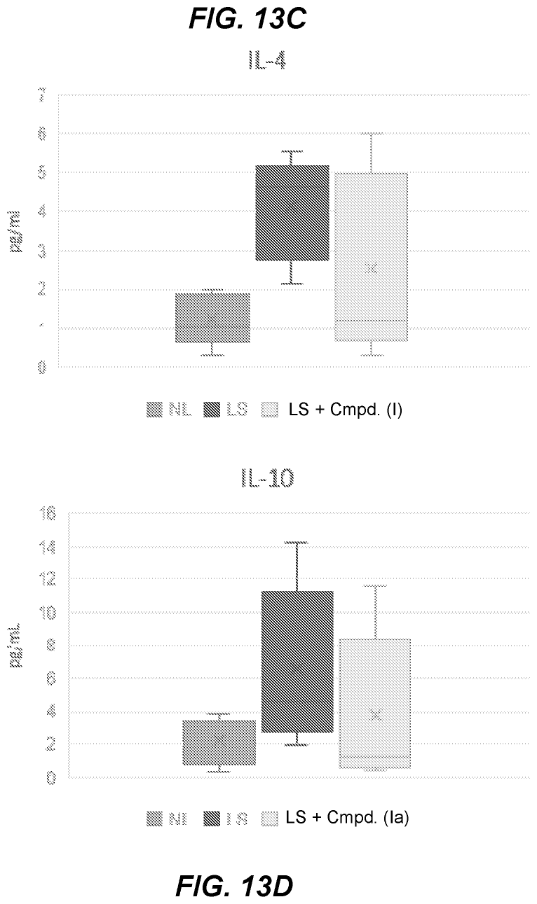

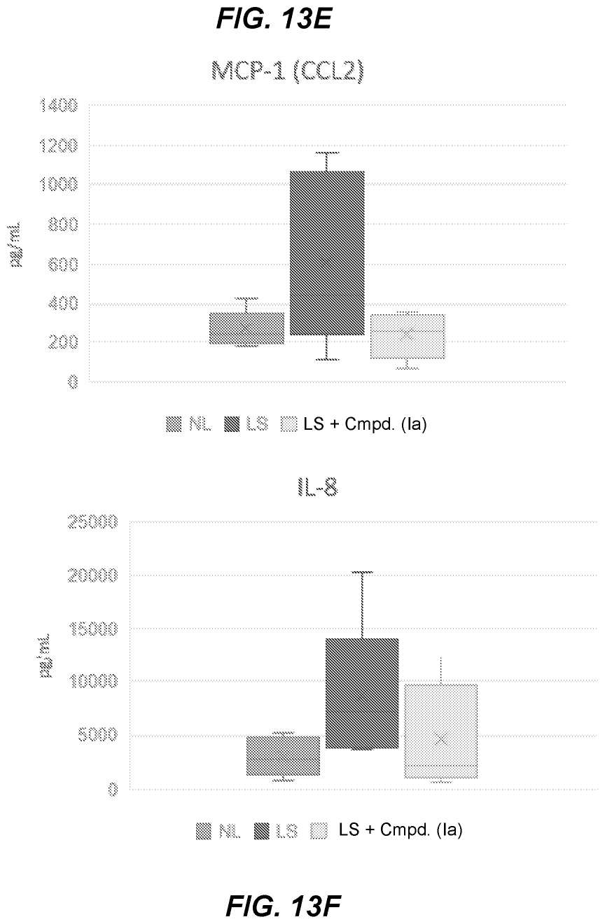

The protein expression analysis was repeated using the MSD platform. Although few of the analytes obtained statistical significance, LS skin treated with the compound of Formula (Ia) had a protein expression profile more similar to the NL profile versus the LS profile. Inflammatory proteins that exhibited decreased expression in LS skin treated with the compound of Formula (Ia) include IL-5 (FIG. 13A; p=0.2), IL-6 (FIG. 13B; p=0.13), IL-4 (FIG. 13C; p=0.12), IL-10 (FIG. 13D; **p=0.002), MCP-1 (FIG. 13E, p=0.09), and IL-8 (FIG. 13F; p=0.056).

In summary, an acute (24 hr) treatment of a LS papulopustular rosacea biopsy with an IRAK4/TrkA inhibitor (the compound of Formula (Ia)) reduced expression of many inflammatory proteins associated with rosacea pathogenesis.

All of the U.S. patents, U.S. patent application publications, U.S. patent applications, foreign patents, foreign patent applications and non-patent publications referred to in this specification are incorporated herein by reference in their entireties.

Although the foregoing disclosure has been described in some detail to facilitate understanding, it will be apparent that certain changes and modifications may be practiced within the scope of the appended claims. Accordingly, the described embodiments are to be considered as illustrative and not restrictive, and the present disclosure is not to be limited to the details given herein, but may be modified within the scope and equivalents of the appended claims.

1. A topical composition comprising a dermatologically acceptable excipient and a compound selected from the following: N-(3-(3-(2-hydroxyethyl)-2-oxoimidazolidin-1-yl)-1-methyl-1H-pyrazol-4-yl)-2-(2-((2,2,2-trifluoroethyl)amino)pyridin-4-yl)-1,3-oxazole-4-carboxamide,
2-(2-((2,2-difluoroethyl)amino)pyridin-4-yl)-N-(3-(3-(2-hydroxyethyl)-2-oxoimidazolidin-1-yl)-1-methyl-1H-pyrazol-4-yl)-1,3-oxazole-4-carboxamide,
N-(3-((3S,4S)-4-hydroxy-3-methyl-2-oxopyrrolidin-1-yl)-1-methyl-1H-pyrazol-4-yl)-2-(2-((2,2,2-trifluoroethyl)amino)pyridin-4-yl)-1,3-oxazole-4-carboxamide,
N-(1-methyl-3-(2-oxoimidazolidin-1-yl)-1H-pyrazol-4-yl)-2-(2-((2,2,2-trifluoroethyl)amino)pyridin-4-yl)-1,3-oxazole-4-carboxamide,
N-(1-methyl-3-((3S)-3-methyl-2-oxopyrrolidin-1-yl)-1H-pyrazol-4-yl)-2-(2-((2,2,2-trifluoroethyl)amino)pyridin-4-yl)-1,3-oxazole-4-carboxamide,
2-(2-((cyclopropylmethyl)amino)pyridin-4-yl)-N-(3-(3-(2-hydroxyethyl)-2-oxoimidazolidin-1-yl)-1-methyl-1H-pyrazol-4-yl)-1,3-oxazole-4-carboxamide,
N-(3-(3,3-dimethyl-4-((methylamino)methyl)-2-oxopyrrolidin-1-yl)-1-methyl-1H-pyrazol-4-yl)-2-(2-((2,2,2-trifluoroethyl)amino)pyridin-4-yl)-1,3-oxazole-4-carboxamide,
N-(3-(3,3-dimethyl-4-((methylamino)methyl)-2-oxopyrrolidin-1-yl)-1-methyl-1H-pyrazol-4-yl)-2-(2-((2,2,2-trifluoroethyl)amino)pyridin-4-yl)-1,3-oxazole-4-carboxamide,
N-(3-(3-isopropyl-2-oxoimidazolidin-1-yl)-1-methyl-1H-pyrazol-4-yl)-2-(2-((2,2,2-trifluoroethyl)amino)pyridin-4-yl)-1,3-oxazole-4-carboxamide,
N-(3-(4-((dimethylamino)methyl)-3,3-dimethyl-2-oxopyrrolidin-1-yl)-1-methyl-1H-pyrazol-4-yl)-2-(2-((2,2,2-trifluoroethyl)amino)pyridin-4-yl)-1,3-oxazole-4-carboxamide,
N-(3-(3-(2-hydroxypropyl)-2-oxoimidazolidin-1-yl)-1-methyl-1H-pyrazol-4-yl)-2-(2-((2,2,2-trifluoroethyl)amino)pyridin-4-yl)-1,3-oxazole-4-carboxamide,
N-(3-(4-((dimethylamino)methyl)-3,3-dimethyl-2-oxopyrrolidin-1-yl)-1-methyl-1H-pyrazol-4-yl)-2-(2-((2,2,2-trifluoroethyl)amino)pyridin-4-yl)-1,3-oxazole-4-carboxamide,
N-(3-(4-((cyclopropylamino)methyl)-3,3-dimethyl-2-oxopyrrolidin-1-yl)-1-methyl-1H-pyrazol-4-yl)-2-(2-((2,2,2-trifluoroethyl)amino)pyridin-4-yl)-1,3-oxazole-4-carboxamide, and
N-(3-(3,3-dimethyl-2-oxopyrrolidin-1-yl)-1-(oxetan-3-yl)-1H-pyrazol-4-yl)-2-(2-((2,2,2-trifluoroethyl)amino)pyridin-4-yl)-1,3-oxazole-4-carboxamide,
or a stereoisomer, tautomers, or pharmaceutically acceptable salts thereof.

2. The topical composition of claim 1, wherein the composition is in the form of a cream, a gel, a spray or an ointment.

3. The topical composition of claim 1, wherein the compound of Formula (I) is at a concentration of about 0.001 wt. % to about 10 wt. %.

4. A method for treating a human having a dermatological condition characterized by inflammation, the method comprising topically administering a therapeutically effective amount of a topical composition according to claim 1.

5. The method of claim 4 wherein the dermatological condition is selected from rosacea, psoriasis, atopic dermatitis, hidradenitis suppurativa, seborrheic dermatitis, contact dermatitis, urticaria, dermatitis herpetiformis, nummular dermatitis, lichen planus, pityriasis rosea, cutaneous lupus, acne, cancers of the skin (e.g. cutaneous T-cell lymphoma), and miliaria.

6. The method of claim 4 wherein the dermatological disorder is rosacea.

7. The method of claim 6 wherein the rosacea is papulopustular rosacea.

8. The method of claim 4 wherein the dermatological disorder is psoriasis.

9. The method of claim 4 wherein the dermatological disorder is atopic dermatitis.

10. The method of claim 4 wherein the dermatological disorder is hidradenitis suppurativa.

11. A method of treating mammalian skin having an inflammatory dermatological disorder, the method comprising administering to the mammalian skin a therapeutically effective amount of a topical composition of claim 1.

12. The method of claim 11, wherein the inflammatory dermatological disorder is rosacea.

13. The method of claim 12, wherein the rosacea is papulopustular rosacea.

14. The method of claim 11, wherein the inflammatory dermatological disorder is psoriasis.

15. The method of claim 11, wherein the inflammatory dermatological disorder is atopic dermatitis.

16. The method of claim 11, wherein the inflammatory dermatological disorder is hidradenitis suppurativa.

17. The method of claim 11, wherein inflammatory dermatological disorder is cutaneous lupus.

18. The method of claim 11, wherein inflammatory dermatological disorder is acne.

19. The method of claim 11, wherein inflammatory dermatological disorder is a cancer of the skin (e.g., cutaneous T-cell lymphoma).

20. The method of claim 11 wherein the mammalian skin is human skin.

21. A method for locally reducing inflammation in mammalian skin, the method comprising topically administering to the mammalian skin an effective amount of a topical composition of claim 1.

22. A method of locally reducing inflammation and vascular dysfunction in mammalian skin, the method comprising topically administering to the mammalian skin an effective amount of a topical composition of claim 1.

23. The method of claim 18, wherein the mammalian skin comprises human skin.

24. The method of claim 20, wherein the human skin has an active rosacea lesion.

25. The method of claim 19, wherein the mammalian skin comprises human skin.
